(19) United States
(12) Patent Application Publication
(10) Pub. No.: US 2025/0002962 A1
(43) Pub. Date: Jan. 2, 2025

(54) RNAYLATION

(71) Applicants: Max-Planck-Gesellschaft zur Förderung der Wissenschaften e.V., München (DE); Universität Heidelberg, Heidelberg (DE)

(72) Inventors: Katharina HÖFER, Marburg (DE); Andres JÄSCHKE, Heidelberg (DE)

(21) Appl. No.: 18/293,313

(22) PCT Filed: Apr. 21, 2022
(86) PCT No.: PCT/EP2022/060525

§ 371 Date: Jan. 29, 2024

(30) Foreign Application Priority Data
Jul. 29, 2021 (WO) PCT/EP2021/071295

(51) Int. Cl. C12P 21/00; C07K 14/245; C12N 9/10

(52) U.S. Cl. CPC C12P 21/00; C07K 14/245; C12N 9/1077; C12Y 204/0203; C07K 2319/00

(57) ABSTRACT

The present invention relates to a method for attaching a 5′-nicotinamidnucleobasedinucleotide (NND)-capped nucleic acid sequence to a fusion protein or to a complex, comprising (a) contacting (i) a heterologous fusion protein which comprises a poly(peptide) of interest being fused to a tag, or (ii) a complex wherein a protein is under physiological conditions complexed with a tag with the 5′-NAD-capped nucleic acid sequence and an ADP-ribosyltransferase (ART) under conditions wherein the 5′-NND-capped nucleic acid sequence is covalently attached to the tag, wherein the tag comprises a recognition motif of the ART and preferably comprises or consists of (i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved; (ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved; (iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved; (iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; (v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or (vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.

The present invention relates to a method for attaching a 5′-nicotinamidnucleobasedinucleotide (NND)-capped nucleic acid sequence to a fusion protein or to a complex, comprising (a) contacting (i) a heterologous fusion protein which comprises a poly(peptide) of interest being fused to a tag, or (ii) a complex wherein a protein is under physiological conditions complexed with a tag with the 5′-NND-capped nucleic acid sequence and an ADP-ribosyltransferase (ART) under conditions wherein the 5′-NND-capped nucleic acid sequence is covalently attached to the tag, wherein the tag comprises a recognition motif of the ART and preferably comprises or consists of (i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved; (ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved; (iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved; (iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; (v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or (vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Posttranslational modification (PTM) is a biochemical modification that occurs to one or more amino acids on a protein after the protein has been translated by a ribosome. Protein post-translational modifications (PTMs) increase the functional diversity of the proteome by the covalent addition of functional groups or proteins, proteolytic cleavage of regulatory subunits, or degradation of entire proteins. These modifications include phosphorylation, glycosylation, ubiquitination, nitrosylation, methylation, acetylation, lipidation and proteolysis and influence almost all aspects of normal cell biology and pathogenesis. There are more than 400 different types of PTMs affecting many aspects of protein functions. Such modifications are crucial molecular regulatory mechanisms to regulate diverse cellular processes. These processes have a significant impact on the structure and function of proteins. Disruption in PTMs can lead to the dysfunction of vital biological processes and hence to various diseases.

Therefore, there is an urgent for identifying and understanding further PTMs. This is critical in the study of cell biology and disease treatment and prevention. This need is addressed by the present invention.

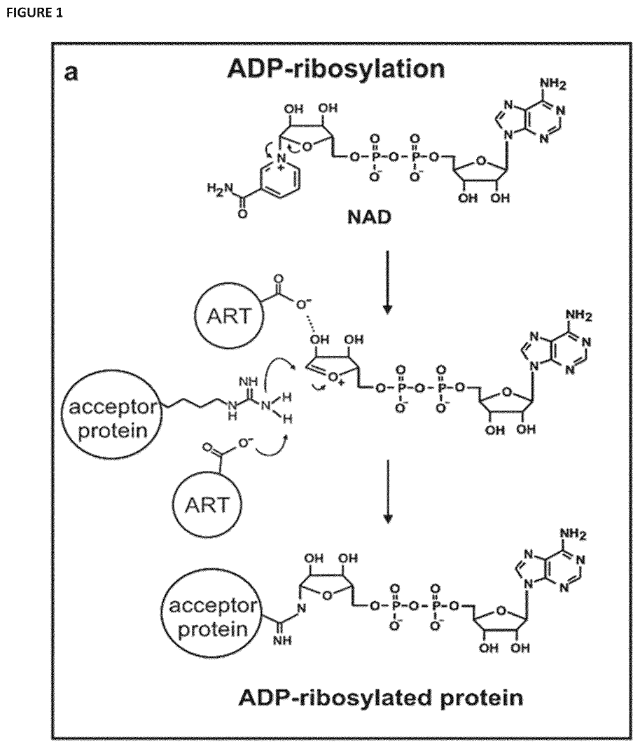

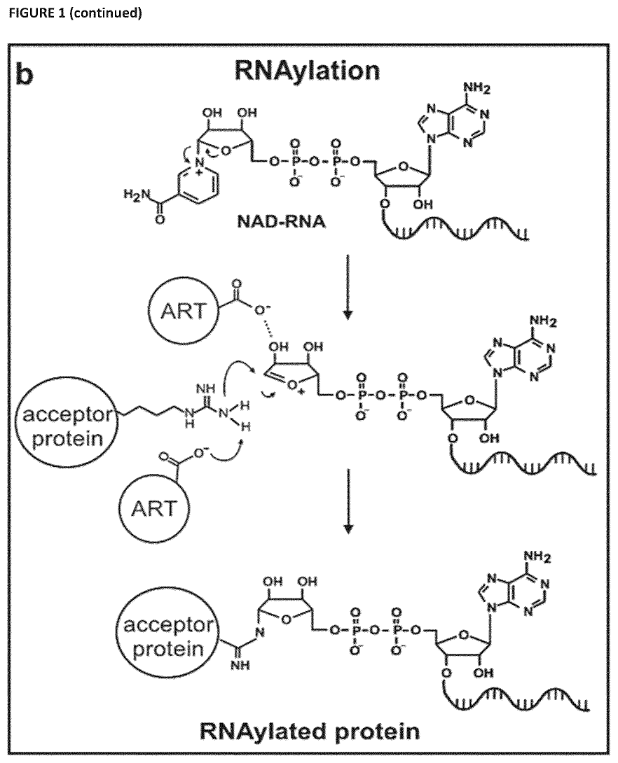

ADP-ribosyl transferases (ARTs) catalyse the transfer of one or multiple ADP-ribose (ADPr) units from nicotinamidadeninedinucleotide (NAD) to target proteins¹. In bacteria and archaea, they act as toxins, are involved in host defence or drug resistance mechanisms⁸, while in eukaryotes, they play roles in distinct processes ranging from DNA damage repair to macrophage activation and stress response⁹. Viruses use ARTs as weapons to reprogram the host's gene expression system⁷. Mechanistically, a nucleophilic group of the target protein (mostly Arg, Glu, Asp, Ser, Cys) attacks the glycosidic carbon atom in the nicotinamide riboside moiety of NAD, forming a covalent bond as N-, O-, or S-glycoside (FIG. 1a)¹.

Here, it is experimentally shown for the first time that bacteriophage T4 ARTs accept not only NAD, but also NND-RNA as substrate, thereby covalently linking entire RNA chains to acceptor proteins in an “RNAylation” reaction. As shown with the ART ModB, ARTs can efficiently RNAylate its host protein target, ribosomal protein S1, at arginine residues and strongly prefers NAD-RNA over NAD. Mutation of a single arginine at position 139 abolishes ADP-ribosylation and RNAylation. It is furthermore shown that ARTs also works with NGD (5′-nicotinamidguanindinucleotide)-RNA, NCD (5′-nicotinamidcytosindinucleotide)-RNA and (5′-nicotinamiduracildinucleotide) NUD-RNA.

These findings reveal the novel PTM “RNAylation”, which is the PTM of protein by the attachment of a NND-nucleic acid sequence via an ART.

Accordingly, the present invention relates in a first aspect to a method for attaching a 5′-nicotinamidnucleobasedinucleotide (NND which is also designated NXD herein)-capped nucleic acid sequence to a fusion protein or to a complex, comprising (a) contacting (i) a heterologous fusion protein which comprises a poly(peptide) of interest being fused to a tag, or (ii) a complex wherein a protein is under physiological conditions complexed with a tag with the 5′-NND-capped nucleic acid sequence and an ADP-ribosyltransferase (ART) under conditions wherein the 5′-NND-capped nucleic acid sequence is covalently attached to the tag, wherein the tag comprises a recognition motif of the ART and preferably comprises or consists of (i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved; (ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved; (iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved; (iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; (v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or (vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.

Nicotinamide adenine dinucleotide (NAD) is a coenzyme central to metabolism. Found in all living cells, NAD is called a dinucleotide because it consists of two nucleotides joined through their phosphate groups. One nucleotide contains an adenine nucleobase and the other nicotinamide. NAD exists in two forms: an oxidized and reduced form, abbreviated as NAD+ and NADH (H for hydrogen), respectively.

The term “nucleic acid sequence” (also called “nucleic acid molecule” herein) in accordance with the present invention includes DNA, such as double or single stranded DNA and RNA. The nucleic acid sequence is preferably single stranded, such as single stranded DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrids molecules, i.e., DNA-DNA, DNA-RNA and RNA-RNA.

The nucleic acid molecule may also be modified by any means known in the art. Non-limiting examples of such modifications include methylation, substitution of one or more of the naturally occurring nucleotides with an analogue, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), alkylators, flourophore (e.g. an Alexa or Cy dye), a fluorescent quencher or biotin. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers.

As will be further detailed herein below, such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1).

Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule considered according to the invention may additionally comprise promoters, enhancers, response elements, signal sequences, polyadenylation sequences, introns, 5′- and 3′-non-coding regions, and the like.

A 5′-nicotinnucleobasedinucleotide (NND)-capped nucleic acid sequence designates a nucleic acid sequence wherein NND is linked to the 5′ end of the nucleic acid sequence via a diphosphate linkage. A nucleobase is a nitrogen-containing biological compound that forms nucleosides. Nucleosides are components of nucleotides, with all of these monomers constituting the basic building blocks of nucleic acids.

The term “protein” (also referred to as “polypeptide”) as used herein interchangeably with the term “polypeptide” describes linear molecular chains of amino acids, including single chain proteins or their fragments, containing at least 50 amino acids. The term “peptide” as used herein describes a group of molecules consisting of up to 49 amino acids. The term “peptide” as used herein describes a group of molecules consisting with increased preference of at least 15 amino acids, at least 20 amino acids at least 25 amino acids, and at least 40 amino acids. The group of peptides and polypeptides are referred to together by using the term “(poly)peptide”. (Poly)peptides may further form oligomers consisting of at least two identical or different molecules. The corresponding higher order structures of such multimers are, correspondingly, termed homo- or heterodimers, homo- or heterotrimers etc. Furthermore, peptidomimetics of such proteins/(poly)peptides where amino acid(s) and/or peptide bond(s) have been replaced by functional analogues are also encompassed by the invention. Such functional analogues include all known amino acids other than the 20 gene-encoded amino acids, such as selenocysteine. The terms “(poly)peptide” and “protein” also refer to naturally modified (poly)peptides and proteins where the modification is effected e.g. by glycosylation, acetylation, phosphorylation and similar modifications which are well known in the art.

The (poly)peptide of interest can be any (poly)peptide for which it is desired to modify the same by the novel PTM RNAylation as disclosed herein. Examples of (poly)peptides of interest will be described herein below.

Both in the fusion protein according to the invention and in the complex according to the invention the (poly)peptide of interest is attached to a tag. The tag can be attached to the N-terminus, the C-terminus of both termini of the (poly)peptide of interest.

In the complex the tag is non-covalently linked (under physiological conditions) to the (poly)peptide of interest, preferably via binding of biotin to avidin, streptavidin or NeutrAvidin. The biotin can be linked to the (poly)peptide of interest and the avidin, streptavidin or NeulrAvidin to the tag or vice versa. Avidin is a protein derived from both avians and amphibians that shows considerable affinity for biotin, a co-factor that plays a role in multiple eukaryotic biological processes. Avidin, Streptavidin and NeutrAvidin, have the ability to bind up to four biotin molecules. The avidin-biotin complex is the strongest known non-covalent interaction (Kd=10⁻¹⁵M) between a protein and a ligand. The bond formation between biotin and avidin is very rapid, and once formed, is unaffected by extremes of pH, temperature, organic solvents and other denaturing agents. Therefore. the indication “under physiological conditions” means that binding must occur under such conditions but does not exclude that binding is also possible under non-physiological conditions. The term “physiological conditions” refers to conditions of the external or internal milieu that may occur in nature for that organism or cell system, in contrast to artificial laboratory conditions.

Protein ADP-ribosylation is an important posttranslational modification that plays versatile roles in multiple biological processes. ADP-ribosylation is catalyzed by a group of enzymes known as ADP-ribosyltransferases (ARTs). Using nicotinamide adenine dinucleotide (NAD) as the donor, it is known from the prior art that ARTs covalently link single or multiple ADP-ribose moieties from NAD to their substrate (poly)peptides, forming mono ADP-ribosylation or poly ADP-ribosylation (PARylation).

In accordance with the present invention a novel function of ARTs has been unexpectedly revealed. ARTs can not only transfer NAD to their substrate (poly)peptides but also a 5′-NND-capped nucleic acid sequence. It is in particular demonstrated in the examples herein below that the ART ModB is capable of covalently linking a 5′-NND-capped nucleic acid sequence to substrate (poly)peptides. It was in particular found that an ADP-ribose-linkage is between RNA and the side chain of an arginine within the substrate (poly)peptide.

It was furthermore found that the substrate (poly)peptides of the ART ModB harbours a motif that is specifically recognized by the ART and that the motif comprises a particular arginine, wherein the side chain of said arginine serves as the site of the linkage of the 5′-NND-capped nucleic acid sequence to the substrate (poly)peptide. This motif can be attached as a tag as described herein above to any (poly)peptide of interest with the consequence that any (poly)peptide of interest can be modified by the novel PTM RNAylation.

For this reason the tag of the invention comprises a recognition motif of the ART and preferably at least one arginine that serves as the site of the linkage of the 5′-NND-capped nucleic acid sequence to the substrate (poly)peptide.

As discussed, RNAylation is illustrated in the examples herein below with the ART ModB. The substrate (poly)peptide of ModB is the protein rS1. The protein rS1 comprises six domain (DI to DVI) and a recognition motif of the ART was found in domains DII and DVI. The wild-type motif within domains DII and DVI corresponds to SEQ ID NOs 1 and 4, respectively. The two wild-type motifs were further investigated and SEQ ID NOs 2 and 5 were identified as the “essential” motifs. The wild-type motives were also proceeded into so-called “vokuhila” motives with a short side and a long side next to the site of conjugation of the 5′-NND-capped nucleic acid sequence.

All these motifs comprise two beta-sheets and a central loop. The loop within domains DII and DVI is shown in SEQ ID NOs 7 and 8, respectively. While the beta sheets are believed to put the loop into position, the amino acids in the loop are recognized by the ART, wherein a conserved and functionally essential arginine (R) serves via its side chain as the site of attachment for the 5′-NND-capped nucleic acid sequence.

Hence, the tag preferably comprises or consists of (i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved; (ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved; (iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved; (iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; (v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or (vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences.

In other terms, using an alignment for two or more sequences or subsequences the percentage of amino acid residues or nucleotides that are the same (e.g. 70%, 75%, 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schaffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences.

As defined herein, sequence identities of at least 80% identical, preferably at least 85% identical, more preferably at least 90% identical, and most preferred at least 95% are envisaged by the invention. However, also envisaged by the invention are with increasing preference sequence identities of at least 97.5%, at least 98.5%, at least 99%, at least 99.5%, at least 99.8%, and 100%.

In accordance with a preferred embodiment of the first aspect of the invention, the nucleobase of the NND is a purine base or a pyrimidine base and is preferably selected from adenine, guanine, cytosine, thymine, and uracil.

Purine bases are preferred as compared to pyrimidine bases.

The five preferred nucleobase adenine (A), cytosine (C), guanine (G), thymine (T), and uracil (U) are called primary or canonical. They function as the fundamental units of the genetic code, with the bases A, G, C, and T being found in DNA while A, G, C, and U are found in RNA. Thymine and uracil are distinguished by the presence or absence, respectively, of a methyl group on the fifth carbon (C5) of these heterocyclic six-membered rings. Adenine and guanine have a fused-ring skeletal structure derived of purine, hence they are member of the class of purine bases. The ring structure of cytosine, uracil, and thymine is derived of pyrimidine, so that they are members of the class of pyrimidine bases.

Among the five preferred nucleobase adenine (A), cytosine (C), guanine (G), thymine (T), and uracil (U) either adenine (A) is preferred because it is the natural substrates of ARTs or cytosine (C), guanine (G), thymine (T), and uracil (U) are preferred because they are non-natural substrate of ARTs. The non-natural substrates are not removed by the humane ADP-ribose hydrolase ARH1 thereby advantageously showing an increase in RNA-protein stability (see Example 7). Among cytosine (C), guanine (G), thymine (T), and uracil (U) the three nucleobases cytosine (C), guanine (G), and uracil (U) are preferred since they form RNA.

Further nucleobases are, for example, xanthine, hypoxanthine, 7-methylguanine, 2,6-diaminopurine, and 6,8-diaminopurine (purine bases), pseudouridine, N1-methyl-pseudouridine or 5,6-dihydrouracil, 5-methyluracil and 5-hydroxymethylcytosins (pyrimidine base).

In accordance with a preferred embodiment of the first aspect of the invention, the ART comprises or consists of SEQ ID NO: 9 or SEQ ID NO: 10 or a sequence being at least 80% identical thereto.

SEQ ID NO: 9 is the amino acid sequence of the ART ModB from Escherichia virus T4 as deposited under Acc. No. CAA67254.1. SEQ ID NO: 10 is the amino acid sequence of the cloned ModB as used in the examples herein below which comprises in addition a His6 tag that serves for the purification of ModB, for example, after it has been recombinantly produced from an expression vector as described herein below for the heterologous fusion protein.

In accordance with a preferred embodiment of the first aspect of the invention the method comprises prior to step (a) the step (a′) fusing the tag as defined in connection with the first aspect to a poly(peptide) of interest, whereby a heterologous fusion protein which comprises a poly(peptide) of interest being fused to the tag is obtained.

In an alternative preferred embodiment of the first aspect of the invention the method comprises prior to step (a) the step (a′) complexing the tag as defined in connection with the first aspect with a poly(peptide) of interest.

For instance, the nucleic acid sequences encoding the poly(peptide) of interest, the tag and optionally a peptide linker may be introduced in frame in an expression vector in expressible form. The expression vector may then be introduced into a host cell and the host cell can be cultured under conditions wherein the heterologous fusion protein is produced. The heterologous fusion protein may then be isolated from the cells.

As an alternative the poly(peptide) of interest, the tag and optionally a peptide linker may be synthesized via peptide synthesis and linked to form the heterologous fusion protein.

In accordance with a preferred embodiment of the first aspect of the invention the method comprises after step (a) step (b) purifying or isolating the fusion protein or the complex with the attached NND-5′-capped nucleic acid sequence.

Means and methods for the isolation or purification of a protein or peptide are known in the art. The means and methods comprise without limitation method techniques and steps such as ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, in Sambrook, 2001, Molecular Cloning: A laboratory manual, 3rd ed, Cold Spring Harbor Laboratory Press, New York.

The present invention relates in a second aspect to a fusion protein comprising a poly(peptide) of interest being fused to a tag as defined in connection with the first aspect of the invention or to a complex comprising a poly(peptide) of interest being complexed with a tag as defined in connection with the first aspect of the invention.

The definitions and preferred embodiments of the first aspect of the invention as far as being applicable to the second aspect of the invention apply mutatis mutandis to the second aspect of the invention.

Hence, also the fusion protein of the second aspect is a heterologous fusion protein which means that the amino acid sequence of the poly(peptide) does not occur in nature, noting that the tag in nature may be part of the protein rS1.

Similarly, also the complex of the second aspect of the invention is preferably formed by binding of biotin to avidin or streptavidin or NeutrAvidin.

Moreover, also in connection with the second aspect the tag comprises a recognition motif of the ART and preferably comprises or consists of (i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved; (ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved; (iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved; (iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; (v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or (vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.

The present invention also relates in connection with the second aspect to a nucleic acid molecule, preferably a vector encoding the fusion protein of the second aspect.

The term “vector” in accordance with the invention means preferably a plasmid, cosmid, virus, bacteriophage or another vector used e.g. conventionally in genetic engineering which carries the nucleic acid molecule of the invention. The nucleic acid molecule of the invention may, for example, be inserted into several commercially available vectors. Non-limiting examples include prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecule inserted into the vector can e.g. be synthesized by standard methods. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide encoding the polypeptide/protein or fusion protein of the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycine, chloramphenicol, and kanamycin. Specifically-designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

In accordance with a further preferred embodiment of the second aspect of the invention a nucleic acid sequence is covalently attached through nicotinamide nucleobase dinucleotide (NND) at its 5′-end to the tag as defined in connection with the first aspect of the invention, preferably to the side chain of the conserved Arg of the tag.

As discussed herein above, by the method of the invention a 5′-nicotinamidnucleobasedinucleotide (NAD)-capped nucleic acid sequence can be attached to a fusion protein or to a complex as defined in connection with the first aspect and preferably to a conserved Arg of the tag being comprised in the fusion protein or to a complex.

Hence, the fusion protein or complex of the above preferred embodiment preferably can be obtained, is obtainable or is obtained by the method of the first aspect of the invention.

The present invention relates in a third aspect to a composition, preferably a pharmaceutical or diagnostic composition comprising a fusion protein or complex as obtainable by the method of the first aspect or the fusion protein and/or complex of the second aspect.

The definitions and preferred embodiments of the first and second aspect of the invention as far as being applicable to the third aspect of the invention apply mutatis mutandis to the third aspect of the invention.

The term “composition” as used herein refers to a composition comprising at least one fusion protein and/or complex as defined above, or combinations thereof which are also collectively referred in the following as compounds.

In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day. Furthermore, if for example said compound is an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of iRNA agent (e.g., about 4.4×10¹⁶ copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of iRNA agent per kg of body weight. The length of treatment needed to observe changes and the interval following treatment for responses to occur vary depending on the desired effect. The particular amounts may be determined by conventional tests which are well known to the person skilled in the art.

In the pharmaceutical composition as well as the medical uses that will be described herein below the active compound can be the (poly)peptide of interest and/or the nucleic acid sequence on the tag. A non-limiting example of a class of pharmaceutically active (poly)peptides of interest are antibodies. Therapeutic antibodies against several kinds of cancer and autoimmune diseases are commercially available. A non-limiting example of a class of pharmaceutically active nucleic acid sequences are siRNAs, that can be designed in order to silence the expression of virtually any desired gene. It is also possible to combine the favourable characteristics of an antibody and an siRNA. For instance, the antibody may bind to a tissue-specific antigen thereby confining or at least focusing the activity of the siRNA to a particular tissue.

A cosmetic composition according to the invention is for use in non-therapeutic applications. Cosmetic compositions may also be defined by their intended use, as compositions intended to be rubbed, poured, sprinkled, or sprayed on, or otherwise applied to the human body for cleansing, beautifying, promoting attractiveness, or altering the appearance. The particular formulation of the cosmetic composition according to the invention is not limited. Envisaged formulations include rinse solutions, emulsions, creams, milks, gels such as hydrogels, ointments, suspensions, dispersions, powders, solid sticks, foams, sprays and shampoos. For this purpose, the cosmetic composition according to the invention may further comprise cosmetically acceptable diluents and/or carriers. Choosing appropriate carriers and diluents in dependency of the desired formulation is within the skills of the skilled person. Suitable cosmetically acceptable diluents and carriers are well known in the art and include agents referred to in Bushell et al. (WO 2006/053613). Preferred formulations for said cosmetic composition are rinse solutions and creams. Preferred amounts of the cosmetic compositions according to the invention to be applied in a single application are between 0.1 and 10 g, more preferred between 0.1 and 1 g, most preferred 0.5 g. The amount to be applied also depends on the size of the area to be treated and has to be adapted thereto.

In accordance with a preferred embodiment of the afore-described three aspect of the invention the nucleic acids in the nucleic acid sequence are RNA, DNA, PNA, Morpholinos or LNA or combinations thereof and are preferably RNA.

It is demonstrated in the examples herein below that an ART can attach a 5′-nicotinamidnucleobasedinucleotide (NND)-capped RNA sequence to a tag harbouring a recognition site of the tag. For this reason the nucleic acids in the nucleic acid sequence are most preferably RNA.

Since ARTs are not only capable of attaching a 5′-nicotinamidnucleobasedinucleotide (NND)-capped RNA sequence to proteins but also covalently link single or multiple ADP-ribose moieties from NAD to their substrate (poly)peptides it is believed that that ARTs can not only attach a 5′-nicotinamidnucleobasedinucleotide (NND)-capped RNA sequence to tags but 5′-nicotinamidnucelobasedinucleotide (NND)-capped nucleic acids sequences in general.

Next to RNA, DNA, PNA, Morpholinos or LNA are nucleic acids that can be used to form a useful nucleic acid sequence.

PNAs are oligonucleotide analogues in which the sugar-phosphate backbone has been replaced by a pseudopeptide skeleton. They bind DNA and RNA with high specificity and selectivity, leading to PNA-RNA and PNA-DNA hybrids more stable than the corresponding nucleic acid complexes.

LNA is an RNA derivative in which the ribose ring is constrained by a methylene linkage between the 2′-oxygen and the 4′-carbon. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. The locked ribose conformation enhances base stacking and backbone pre-organization. This significantly increases the hybridization properties (melting temperature) of oligonucleotides.

Morpholinos are synthetic uncharged P-chiral analogs of nucleic acids. Morpholino oligonucleotides are typically constructed by linking together 25 subunits, each bearing one of the four nucleic acid bases.

The above types of nucleotides can be combined in one sequence whenever desired. Such sequences can be synthesized chemically or are commercially available.

While the length of the nucleic acid sequence to be used herein is not particularly limited it is preferred that the nucleic acid sequence comprises about 100 or less nucleotides, preferably about 50 or less nucleotides and most preferably about 25 or less nucleotides.

The term “about” herein is with increasing preference ±20%, 10% and ±5%.

In accordance with a further preferred embodiment of the afore-described three aspects of the invention the nucleic acid sequence is an siRNA, shRNA of an antisense molecule.

The nucleic acid sequence is preferably an antisense molecule (such as an antisense oligonucleotide, e.g. an LNA-GapmeR, an Antagomir, or an antimiR), siRNA, shRNA capable of inhibiting the expression of target nucleic acid molecule, typically an mRNA being expressed in host or organism (such as a human). Such nucleic acid sequences may comprise DNA sequences (e.g. LNA GapmeRs) or RNA sequences (e.g. siRNAs). As will be also further detailed herein below, nucleotide-based compounds inhibiting the expression of a target nucleic acid molecule may be single stranded (e.g. LNA GapmeRs) or double-stranded (e.g. siRNAs).

The antisense technology for the downregulation of target nucleic acid molecule is well-established and widely used in the art to treat various diseases. The basic idea of the antisense technology is the use of oligonucleotides for silencing a selected target RNA through the exquisite specificity of complementary-based pairing (Re, Ochsner J., 2000 October; 2(4): 233-236). Herein below details on the antisense construct compound classes of siRNAs, shRNAs and antisense oligonucleotides are provided. As will be further detailed herein below, antisense oligonucleotides are single stranded antisense constructs while siRNAs and shRNAs are double stranded antisense constructs with one strand comprising an antisense oligonucleotide sequence being (i.e. the so-called antisense strand). All these compound classes may be used to achieve downregulation or inhibition of a target RNA.

The term “siRNA” in accordance with the present invention refers to small interfering RNA, also known as short interfering RNA or silencing RNA. siRNAs are a class of 12 to 30, preferably 18 to 30, more preferably 20 to 25, and most preferred 21 to 23 or 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome. siRNAs have a well defined structure: a short double-strand of RNA (dsRNA), advantageously with at least one RNA strand having an overhang. Each strand typically has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Thus, any gene of which the sequence is known can in principle be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Also preferably at least one RNA strand has a 5′- and/or 3′-overhang. Preferably, one or both ends of the double-strand have a 3′-overhang from 1-5 nucleotides, more preferably from 1-3 nucleotides and most preferably 2 nucleotides. In general, any RNA molecule suitable to act as siRNA is envisioned in the present invention. The most efficient silencing was so far obtained with siRNA duplexes composed of 21-nt sense and 21-nt antisense strands, paired in a manner to have 2-nt 3′-overhangs. The sequence of the 2-nt 3′ overhang makes a small contribution to the specificity of target recognition restricted to the unpaired nucleotide adjacent to the first base pair (Elbashir et al. Nature. 2001 May 24; 411(6836):494-8). 2′-deoxynucleotides in the 3′ overhangs are as efficient as ribonucleotides, but are often cheaper to synthesize and probably more nuclease resistant. The siRNA according to the invention comprises an antisense strand which comprises or consists of a sequence which is with increasing preference complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, or at least 21 nucleotides of the target nucleic acid sequence.

A preferred example of a siRNA is an Endoribonuclease-prepared siRNA (esiRNA). An esiRNA is a mixture of siRNA oligos resulting from cleavage of a long double-stranded RNA (dsRNA) with an endoribonuclease such as Escherichia coli RNase III or dicer. esiRNAs are an alternative concept to the usage of chemically synthesized siRNA for RNA Interference (RNAi). For the generation of esiRNAs a cDNA of an lncRNA template may be amplified by PCR and tagged with two bacteriophage-promotor sequences. RNA polymerase is then used to generate long double stranded RNA that is complimentary to the target-gene cDNA. This complimentary RNA may be subsequently digested with RNase III from Escherichia coli to generate short overlapping fragments of siRNAs with a length between 18-25 base pairs. This complex mixture of short double stranded RNAs is similar to the mixture generated by dicer cleavage in vivo and is therefore called endoribonuclease-prepared siRNA or short esiRNA. Hence, esiRNA are a heterogeneous mixture of siRNAs that all target the same mRNA sequence. esiRNAs lead to highly specific and effective gene silencing.

A “shRNA” in accordance with the present invention is a short hairpin RNA, which is a sequence of RNA that makes a (tight) hairpin turn that can also be used to silence gene expression via RNA interference. shRNA preferably utilizes the U6 promoter for its expression. The shRNA hairpin structure is cleaved by the cellular machinery into siRNA, which is then bound to the RNA-induced silencing complex (RISC). This complex binds to and cleaves mRNAs which match the shRNA that is bound to it. The shRNA according to the invention comprises an antisense strand which comprises or consists of a sequence which is with increasing preference complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides of the target nucleic acid sequence.

The term “antisense oligonucleotide” in accordance with the present invention refers to a single-stranded nucleotide sequence being complementary by virtue of Watson-Crick base pair hybridization to the target nucleic acid sequence whereby the target nucleic acid sequence is blocked. The antisense oligonucleotides may be unmodified or chemically modified. In general, they are relatively short (preferably between 13 and 25 nucleotides). Moreover, they are specific for the target nucleic acid sequence, i.e. they hybridize to a unique sequence in the total pool of targets present in the target cells/organism. The antisense oligonucleotide according to the invention comprises or consists a sequence which is with increasing preference complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides of the target nucleic acid sequence.

The antisense oligonucleotide is preferably a LNA-GapmeR, an Antagomir, or an antimiR.

LNA-GapmeRs or simply GapmeRs are potent antisense oligonucleotides used for highly efficient inhibition of mRNA and lncRNA function. GapmeRs function by RNase H dependent degradation of complementary RNA targets. They are an excellent alternative to siRNA for knockdown of mRNA and lncRNA. They are advantageously taken up by cell without transfection reagents. GapmeRs contain a central stretch of DNA monomers flanked by blocks of LNAs. The GapmeRs are preferably 14-16 nucleotides in length and are optionally fully phosphorothioated. The DNA gap activates the RNAse H-mediated degradation of targeted RNAs and is also suitable to target transcripts directly in the nucleus. The LNA-GapmeR according to the invention comprises a sequence which is with increasing preference complementary to at least 13 nucleotides, at least 14 nucleotides, or at least 15 nucleotides of the target nucleic acid sequence.

As mentioned, AntimiRs are oligonucleotide inhibitors that were initially designed to be complementary to a miRNA. AntimiRs against miRNAs have been used extensively as tools to gain understanding of specific miRNA functions and as potential therapeutics. As used herein, the AntimiRs are designed to be complementary to the target nucleic acid sequence. AntimiRs are preferably 14 to 23 nucleotides in length. An AntimiR according to the invention more preferably comprises or consists of a sequence which is with increasing preference complementary to at least 15 nucleotides, at least 16 nucleotides, at least 17 nucleotides, at least 18 nucleotides, at least 19 nucleotides, at least 20 nucleotides, at least 21 nucleotides, at least 22 nucleotides, or at least 23 nucleotides of the target nucleic acid sequence.

AntimiRs are preferably AntagomiRs. AntagomiRs are synthetic 2-O-methyl RNA oligonucleotides, preferably of 21 to 23 nucleotides which are preferably fully complementary to the selected target nucleic acid sequence. While AntagomiRs were initially designed against miRNAs they may also be designed against mRNAs. The AntagomiRs according to the invention therefore preferably comprises a sequence being complementary to 21 to 23 nucleotides of the target nucleic acid sequence. AntagomiRs are preferably synthesized with 2′-OMe modified bases (2′-hydroxyl of the ribose is replaced with a methoxy group), phosphorothioate (phosphodiester linkages are changed to phosphorothioates) on the first two and last four bases, and an addition of cholesterol motif at 3′ end through a hydroxyprolinol modified linkage. The addition of 2′-OMe and phosphorothioate modifications improve the bio-stability whereas cholesterol conjugation enhances distribution and cell permeation of the AntagomiRs.

Antisense molecules (including antisense oligonucleotides, such as LNA-GapmeR, an Antagomir, an antimiR), siRNAs and shRNAs useful in accordance with the present invention are preferably chemically synthesized using a conventional nucleic acid synthesizer. Suppliers of nucleic acid sequence synthesis reagents include Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK).

The ability of antisense molecules (including antisense oligonucleotides, such as LNA-GapmeR, an Antagomir, an antimiR), siRNA, and shRNA to potently, but reversibly, silence or inhibit a target nucleic acid sequence in vivo makes these molecules particularly well suited for use in the pharmaceutical composition of the invention.

The antisense molecules (including antisense oligonucleotides, such as LNA-GapmeR, an Antagomir, an antimiR), siRNAs, shRNAs may comprise modified nucleotides such as locked nucleic acids (LNAs).

In accordance with another preferred embodiment of the afore-described three aspects of the invention the nucleic acid sequence comprises a fluorescent label at its 3′-end, preferably Alexa Fluor or Cy dye or comprises biotin at its 3′-end.

A fluorescent label is particularly advantageous in the diagnostic composition of the invention since thereby the fusion or complex of the invention with the attached nucleic acid sequence can be located in vivo with a subject (preferably a human subject).

The Cy dye is preferably Cy2, Cy3, or Cy5. The Alex Fluor is preferably Alexa Fluor 488, 532, 546, 555, 568, 594, 647, 660, 680, 700 and 750.

The biotin at the 3′-end of the nucleic acid sequence has to be held distinct from the biotin that may be present in the complex according to the invention. The biotin at the 3′-end of the nucleic acid sequence can serve as a further site of attachment via avidin or streptavidin or NeutrAvidin, this time to the nucleic acid sequence and not for linking the tag and (poly)peptide of interest.

In accordance with a further preferred embodiment of the afore-described three aspects of the invention the nucleic acid sequence comprises a moiety that can be used in click-chemistry, such as an alkyne or azide.

“Click-chemistry is an art-established term; see e.g. Kolb et al. (2001) Click chemistry: diverse chemical function from a few good reactions. Angew. Chem. Int. Ed. 40 (11):2004; Sletten et al. (2009) Bioorthogonal Chemistry: Fishing for Selectivity in a Sea of Functionality. Angew. Chem. Int. Ed. 48:6998; Jewett et al. (2010) Cu-free click cycloaddition reactions in chemical biology. Chem. Soc. Rev. 39(4):1272; Best et al. (2009) Click Chemistry and Bioorthogonal Reactions: Unprecedented Selectivity in the Labeling of Biological Molecules. Biochemistry. 48:6571; and Lallana et al. (2011) Reliable and Efficient Procedures for the Conjugation of Biomolecules through Huisgen Azide-Alkyne Cycloadditions. Angew. Chem. Int. Ed. 50:8794. While there are a number of reactions that fulfill the criteria, the Huisgen 1,3-dipolar cycloaddition of azides and terminal alkynes has emerged as the frontrunner.

In accordance with a preferred embodiment of the afore-described three aspects of the invention the poly(peptide) of interest is an antibody, antibody mimetic, cytokine, interleukin, transmembrane protein, membrane-anchored protein, an enzyme or a DNA and/or RNA-binding proteins.

The term “antibody” as used in accordance with the present invention comprises, for example, polyclonal or monoclonal antibodies. Furthermore, also derivatives or fragments thereof, which still retain the binding specificity to the target, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on the (poly)peptide of interest of the invention. The second antigen may, for example, be a tumor marker that is specifically expressed on cancer cells or a certain type of cancer cells. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Kohler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope. Surface plasmon resonance as employed in the BIAcore system can be used to increase the efficiency of phage antibodies.

As used herein, the term “antibody mimetics” refers to compounds which, like antibodies, can specifically bind antigens, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers®, trispecific binding molecules and prododies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity can be selected, for example, by phage display techniques. The binding specificity of the different A-domains contained in an avimer may but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule by randomising the amino acids on the binding surface (Mouratou B, Behar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. The trispecific binding molecule is preferably a TriTac. A TriTac is a T-cell engager for solid tumors which comprised of three binding domains being designed to have an extended serum half-life and be about one-third the size of a monoclonal antibody.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects.

The cytokine is preferably selected from the group consisting of IL-2, IL-12, TNF-alpha, IFN alpha, IFN beta, IFN gamma, IL-10, IL-15, IL-24, GM-CSF, IL-3, IL-4, IL-5, IL-6, IL-7, IL-9, IL-11, IL-13, LIF, CD80, B70, TNF beta, LT-beta, CD-40 ligand, Fas-ligand, TGF-beta, IL-1 alpha and IL-1beta.

The chemokine is preferably selected from the group consisting of IL-8, GRO alpha, GRO beta, GRO gamma, ENA-78, LDGF-PBP, GCP-2, PF4, Mig, IP-10, SDF-1alpha/beta, BUNZO/STRC33, I-TAC, BLC/BCA-1, MIP-1 alpha, MIP-1 beta, MDC, TECK, TARC, RANTES, HCC-1, HCC-4, DC-CK1, MIP-3 alpha, MIP-3 beta, MCP-1-5, Eotaxin, Eotaxin-2, I-309, MPIF-1, 6Ckine, CTACK, MEC, Lymphotactin and Fractalkine.

Enzymes are proteins that act as biological catalysts (biocatalysts). Catalysts accelerate chemical reactions. The molecules upon which enzymes may act are called substrates, and the enzyme converts the substrates into different molecules known as products. Almost all metabolic processes in the cell need enzyme catalysis in order to occur at rates fast enough to sustain life. The enzyme is preferably a sequence-specific DNA or RNA nuclease and the DNA or RNA nuclease is most preferably a Cas nuclease (e.g. Cas 9, Cpf1 or Cms1). The Cas nuclease can cleave specifically at a desired position in the genome of a cell, noting that the exact position is determined by a guide RNA. The guide RNA may be attached to the poly(peptide) of interest as described herein above.

Proteins that bind both DNA and RNA epitomize the ability to perform multiple functions by a single gene product. Such DNA- and RNA-binding proteins (DRBPs) regulate many cellular processes, including transcription, translation, gene silencing, microRNA biogenesis and telomere maintenance. An example are Zinc finger binding proteins. The DNA/RNA-binding protein is preferably one that is responsible for RNA transport and/or localization.

In accordance with a further preferred embodiment of the afore-described three aspects of the invention the poly(peptide) of interest further comprises a purification tag, preferably a His-tag.

Hence, the fusion protein or complex may comprise a purification tag that facilitates its purification. Non-limiting examples of such tags are an ALFA-tag, V5-tag, Myc-tag, HA-tag, Flag-tag, Spot-tag, T7-tag or NE-tag. The His-tag is used in the examples and is therefore preferred.

In accordance with a further preferred embodiment of the afore-described three aspects of the poly(peptide) and the tag are fused via a peptide-linker, preferably a G-linker or a GS-linker.

In the fusion protein the tag is covalently linked by one or more peptide bonds to the (poly)peptide of interest. In the case of only one peptide bond the (poly)peptide of interest and the tag are directly fused to each other.

In accordance with the above preferred embodiment the (poly)peptide of interest and the tag are fused to each other via a linker (comprising two or more peptide bonds), such as a GS-linker or a G-linker. A G-linker is used in the appended examples.

The present invention relates in a fourth aspect to a kit for attaching a 5′-nicotinamidadenindinucleotide (NND)-capped nucleic acid sequence to a (poly)peptide of interest, wherein the kit comprises (a) the tag as defined in connection with the first aspect, (b) an ADP-ribosyltransferase (ART) being capable of covalently attaching a 5′-NND-capped nucleic acid sequence to the tag or a nucleic acid molecule encoding said ART, and (c) optionally instructions how to covalently attach the tag with the ART to the (poly)peptide of interest.

The definitions and preferred embodiments of the first, second and third aspect of the invention as far as being applicable to the fourth aspect of the invention apply mutatis mutandis to the fourth aspect of the invention.

The kit preferably includes a plurality of compartments for the ingredients of the kit with each one of the compartments being filled with one ingredient. The compartments can be, for example, be tubes or vials, bags or other packages.

The ART is preferably provided in glycerol, such as about 50% glycerol. The tag is preferably provided in about 50 mM Tris-HCl (pH7.5), about 300 mM NaCl and about 50% glycerol.

The instructions how to covalently attach the tag with the ART to the (poly)peptide of interest preferably additionally comprise guidance on the reaction conditions to be used in connection with the kit, such as temperature, time and reaction buffer ingredients. Preferred but non-limiting temperature and/or time are about 2 h and about 15° C. A preferred but non-limiting reaction buffer comprises about 50 mM Tris-HCl (pH 7.5), about 10 mM Mg(OAc)2, about 22 mM NH₄Cl, about 1 mM EDTA, about 10 mM β-mercaptoethanol, about 1% glycerol, about 1 μM ADP-ribosyltransferase (ART, e.g. ModB), about 0.1-10 μM (poly)peptide, and about 1-10 μM 5′-NND-capped nucleic acid sequence-. These reaction conditions may also be applied in connection with the method of the first aspect of the invention.

The instructions can be in the from or leaflet being packed as a part of the kit but can also be in the form of a weblink or QR code that directs to instructions as stored on the internet.

In accordance with a preferred embodiment of the fourth aspect, the kit further comprises a reaction buffer or buffer stock solution, preferably wherein the reaction buffer or the final reaction buffer to be prepared from the buffer stock solution comprises
Mg(OAc)₂ at a concentration of 50-200 mM, preferably about 100 mM;
NH₄Cl at a concentration of 100-500 mM, preferably about 220 mM;
Tris-acetate pH 7.5 at a concentration of 250-1000 mM, preferably about 500 mM;
EDTA at a concentration of 5-15 mM, preferably about 10 mM;
β-mercaptoethanol at a concentration of 50-200 mM, preferably about 100 mM; and
glycerol at a concentration of 5-15%, preferably about 10%.

In the case of a buffer stock solution within kit, the instructions how to covalently attach the tag with the ART to the (poly)peptide of interest preferably additionally comprise information on the dilution of the stock solution in order to obtain the reaction buffer from the buffer stock solution. Non-limiting examples of buffer stock solution are a 5× buffer stock solution and a 10× buffer stock solution A reaction buffer in accordance with the above preferred embodiment is used in the appended examples for the ART ModB and was found to work very well for this enzyme when used in the method of the invention. For this reason the reaction buffer in accordance with the above preferred embodiment of the kit is also preferably used for the method of the first aspect of the invention.

In accordance with a preferred embodiment of the fourth aspect the kit further comprises
one or more of MgCl₂ at least 0.25 M, preferably at a concentration of 0.5 M to 2 M, most preferably about 1 M.

Imidazolide nicotinamide mononucleotide (Im-NMN), nuclease free water, and a positive control, preferably an oligonucleotide that comprises at its 3′-end a fluorescent label and/or a control fusion protein comprising a control poly(peptide) being fused to or complexed with a tag as connection with the first aspect of the invention.

In case MgCl₂ is comprised in the kit the instructions preferably comprise in addition the information that when using the kit the concentration of EDTA should not be higher as the concentration of Mg²⁺ ions. This is because EDTA forms a complex with Mg²⁺, which is not accessible for enzymes.

In case Im-NMN is comprised in the kit the instructions preferably inform that when using the kit the Im-NMN is to be used in at least 1000× fold excess over 5′-nicotinamidnucleobasedinucleotide (NND)-capped nucleic acid sequence.

Nuclease free water is in particular comprised in the kit in case the kit comprises a buffer stock solution. Nuclease free water is then used to prepare the final buffer solution.

The positive control can be used in order check whether the kit components per se work in case the kit is used in a method for attaching a 5′-nicotinamidnucleobasedinucleotide (NND)-capped nucleic acid sequence to a fusion protein or to a complex as described herein above, but as a result the desired RNAylated fusion protein or complex is not obtained. A possible reason for such failure may be, for example, that the tag was not successfully attached to the (poly)peptide or interest.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show:

FIG. 1: Mechanisms of ADP-ribosylation and proposed “RNAylation”. a. Here, the mechanism of ADP-ribosylation is shown exemplarily for arginine. Initially, the N-glycosidic bond between the ribose and nicotinamide is destabilised by a glutamate residue of an ART. This leads to the formation of an oxocarbenium ion of ADP-ribose. Nicotinamide serves as the leaving group. This electrophilic ion is attacked by a nucleophilic arginine residue of the acceptor protein after glutamate-mediated proton abstraction. This leads to the formation of an N-glycosidic bond³⁰. b. Analogous to ADP-ribosylation in the presence of NAD, we propose that ARTs might use NAD-RNA to catalyse an “RNAylation” reaction, thereby covalently attaching an RNA to an acceptor protein.

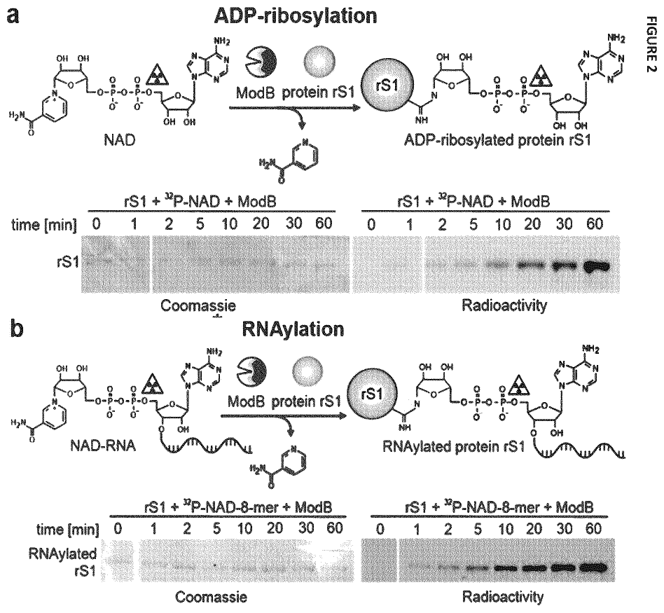

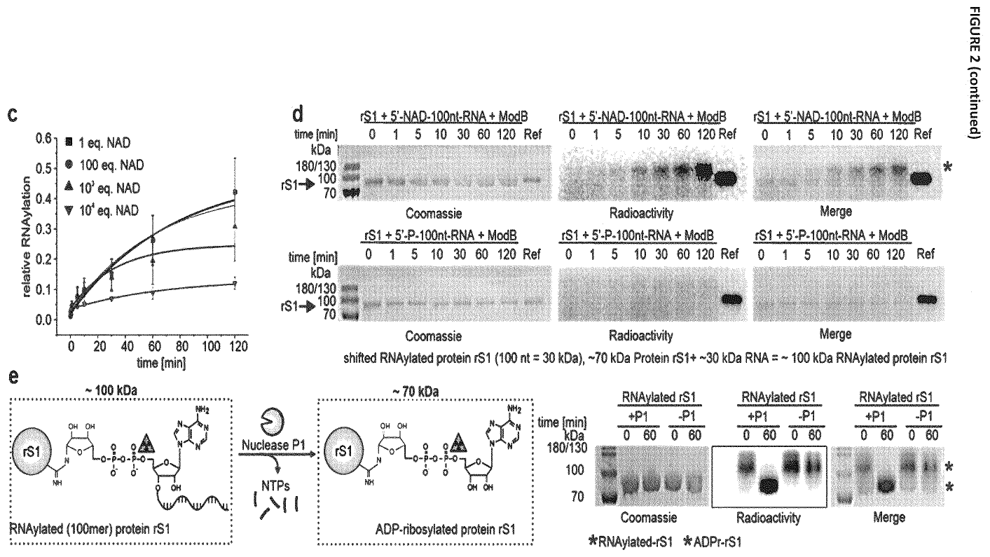

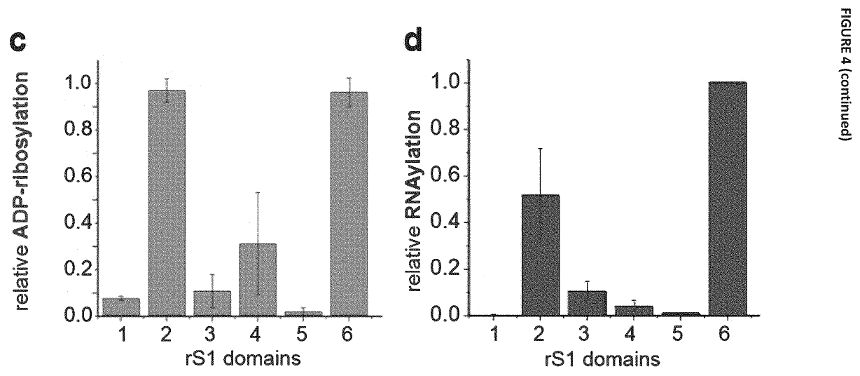

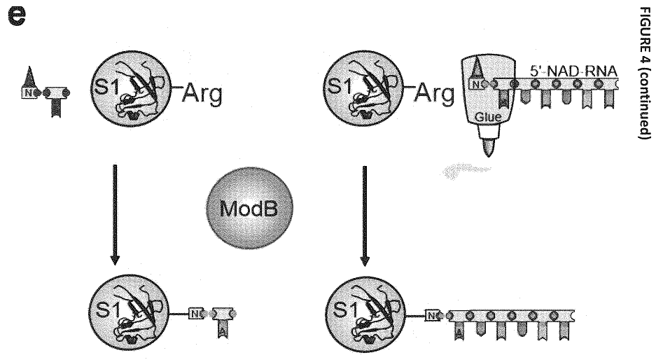

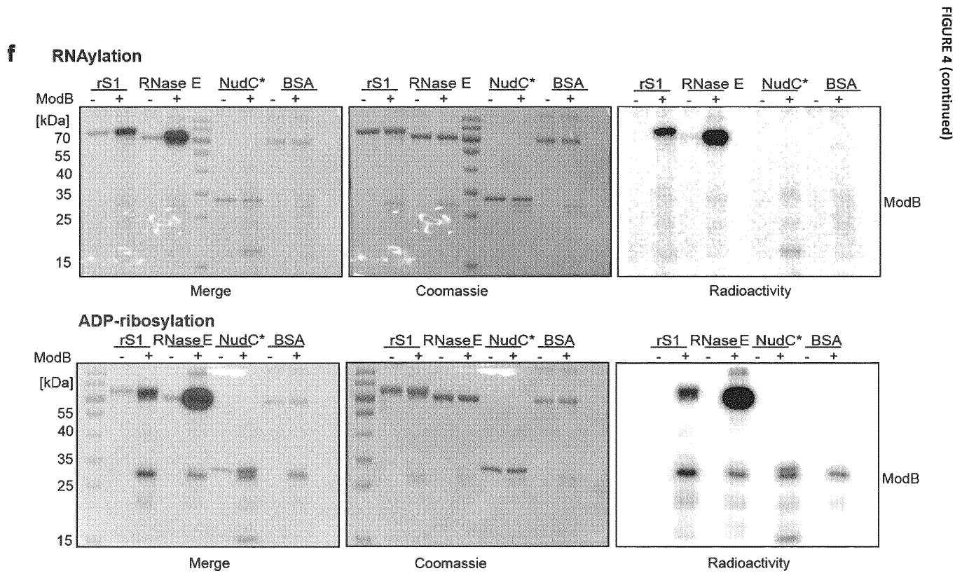

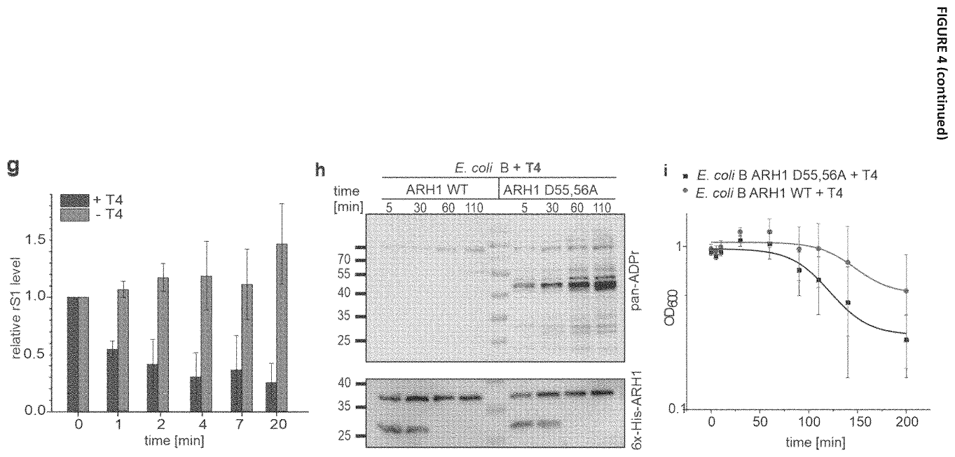

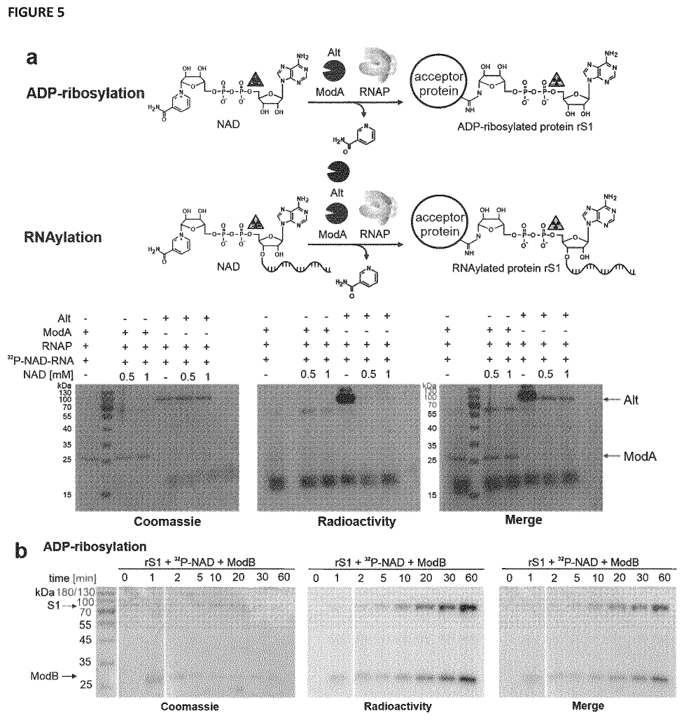

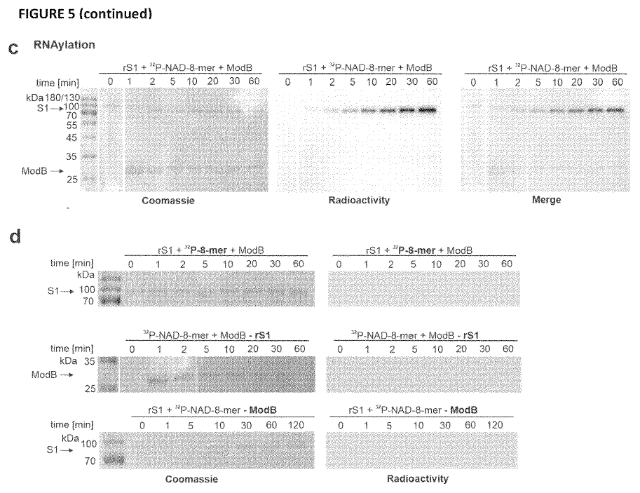

FIG. 2: Post-translational protein modification of rS1 by the ART ModB in vitro. a, Time course of the ADP-ribosylation of rS1 by ModB (complete SDS-PAGE gels are shown in FIG. 5b). b, Time course of the RNAylation of rS1 by ModB (complete SDS-PAGE gels are shown in FIG. 5c). c, in vitro kinetics of the RNAylation of rS1 by ModB in the presence of excess NAD d, in vitro kinetics of the RNAylation of rS1 by ModB using 5′-NAD-100 nt-RNA (Qβ-RNA) as substrate (top panel), analysed by SDS-PAGE. Shifted RNAylated rS1 is highlighted with a pink asterisk. 5′-P-100-nt-RNA is used as a negative control (bottom panel). e, Nuclease P1 digest of RNAylated protein rS1. The covalently attached 100 nt long RNA results in a shift of the RNAylated protein rS1 (˜100 kDa) in SDS-PAGE. Treatment of the RNAylated protein rS1 with nuclease P1, which cleaves the phosphodiester bond, resulting in degradation of the attached RNA into mononucleotides. Nuclease P1 can covert RNAylated rS1 into ADP-ribosylated rS1 (˜70 kDa), which can be visualised by a downshifted protein band on the SDS-PAGE gel.

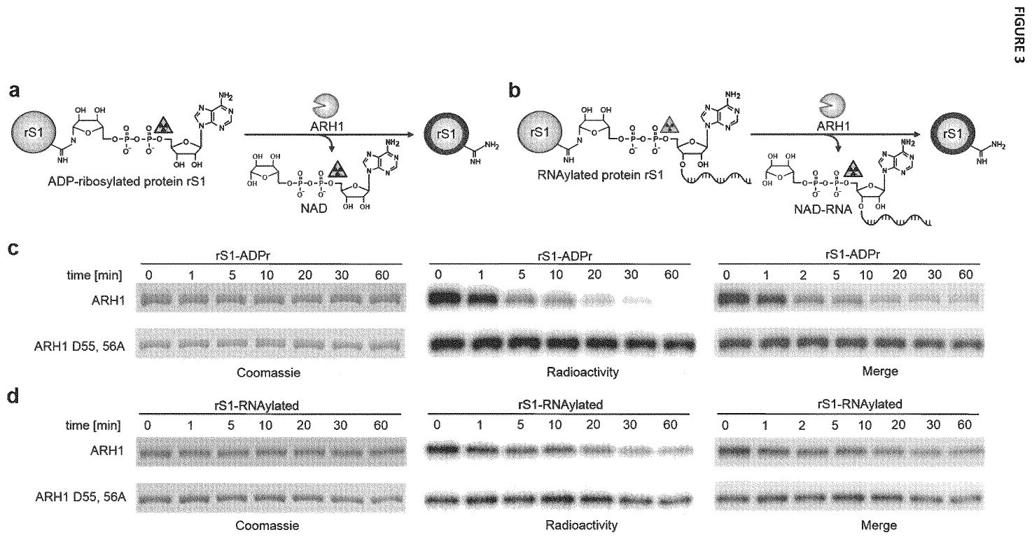

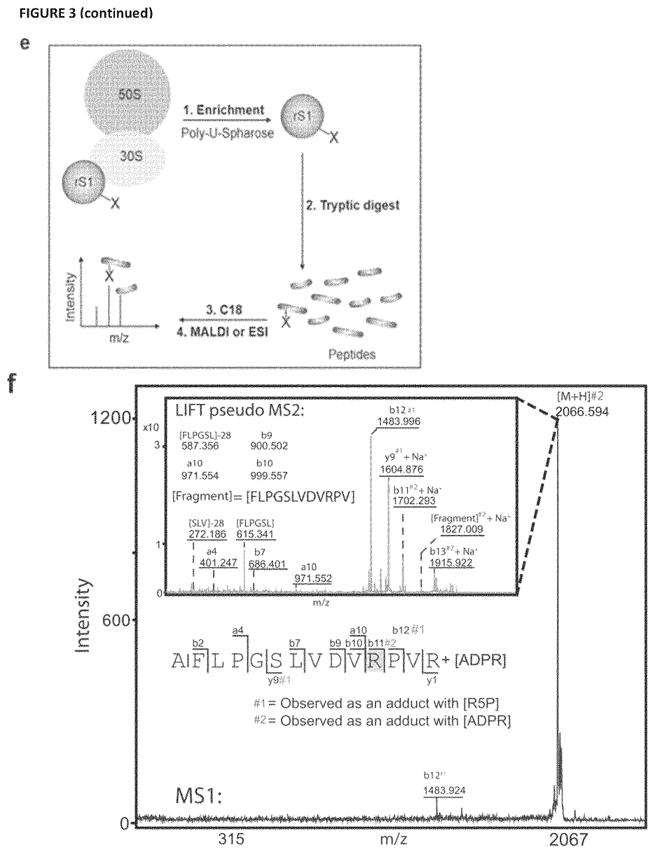

FIG. 3: Identification of RNAylation sites of rS1. a-d, Specific removal of ADP-ribosylation and RNAylation by ARH1. Enzyme kinetics of ARH1 in the presence of ADP-ribosylated or RNAylated protein rS1 analysed by SDS-PAGE. e, a pipeline for identifying the modified amino acid residue by mass spectrometry f, MALDI-TOF-MS of in vitro modified protein rS1. Isolation scan (MS1) and pseudo MS2 (LIFT) spectrum of a peptide-ADPR conjugate. The given peptide AFLPGSLVDVRPVR (SEQ ID NO: 11) would result in a peak at 2067 if it is linked to ADPR. MALDI-TOF-MS with in vitro modified protein rS1 resulted in the depicted two spectra. LIFT parent ion isolation resulted in the given MS1 with little interference. Note: The shifted b12 ion at m/z=1483 Th that corresponds to a peptide with R5P modification, indicating the fragile nature of ADP-ribosylations. The resulting pseudo MS2 yields sufficient sequencing ions to confirm the peptide sequence as well as the ADPr modification on the arginine residue boxed in yellow.

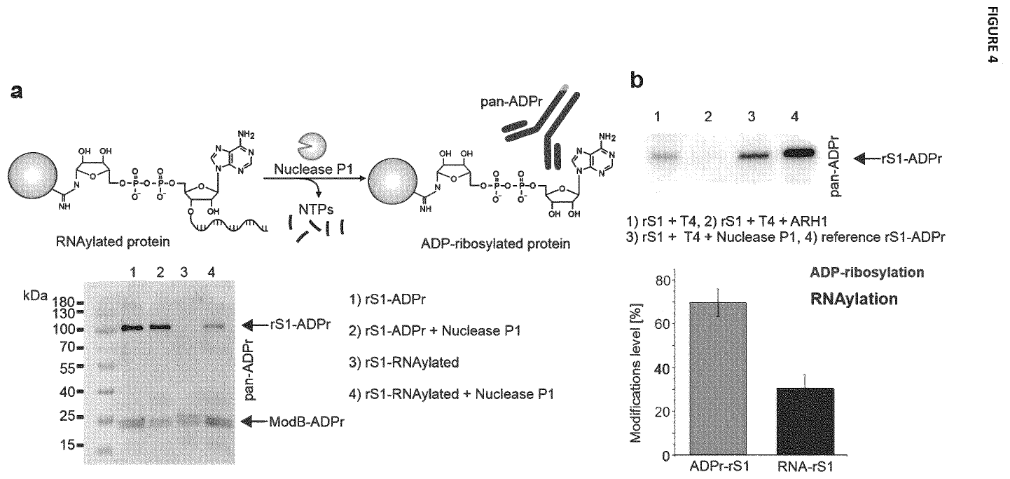

FIG. 4: in vivo characterisation of ADP-ribosylation and RNAylation. a, Illustration of the quantification of protein rS1 RNAylation using a nuclease P1 digest and Western Blot analysis. b, Quantification of rS1 RNAylation in vivo. c, Quantification of ADP-ribosylation and d, RNAylation. Modification of rS1 domains 1-6. n=2 of biologically independent replicates. e, Graphical illustration of ADP-ribosylation and RNAylation of proteins carrying a S1-motif by ModB. f, SDS-PAGE analysis of the RNAylation and ADP-ribosylation of protein rS1, RNase E, inactive NudC mutant (* V157A, E174A, E177A, E178A) and BSA by ModB. n=2 of biologically independent replicates. g, Quantification of rS1 levels in the presence (+T4) or absence of T4 (−T4), n=4. h, ARH1-mediated removal of ADP-ribosylation and RNAylation modifications during T4 infection. i, Time course of bacteriophage T4-mediated lysis of E. coli expressing a plasmid-borne copy of ARH1-WT or its inactive mutant ARH1 D55,56A.

FIG. 5: ADP-ribosylation and RNAylation by T4 ARTs. a, Functional characterisation of the ARTs Alt and ModA. Self- and target modification by Alt using NAD or NAD-RNA analysed by SDS-PAGE and autoradiography. b, Time course of the ADP-ribosylation of rS1 by ModB and c, Time course of the RNAylation of rS1 by ModB analysed by SDS-PAGE. d, Negative controls for RNAylation of rS1 with ModB. RNAylation assay was performed in the presence of ³²P-RNA, in the absence of rS1 (−rS1) or ModB (−ModB).

FIG. 6: Characterisation of the RNAylation of protein rS1 by ModB. a, Inhibition of in vitro RNAylation of protein rS1 by ModB via ART inhibitor 3-methoxybenzamide (3-MB). Reactions were performed with ³²P-NAD-RNA 8-mer (³²P-NAD-8-mer) as well as ³²P-RNA 8-mer (negative control). b, in vitro digest of RNAylated and ADP-ribosylated protein rS1 by RNase T1. Reactions performed in the absence of RNase T1 (−) serve as negative controls. Protein rS1 ADP-ribosylated in the presence of ³²P-NAD applied as reference (S1-ADPr) c, in vitro treatment of ADP-ribosylated and RNAylated protein rS1 with NudC (marked with an arrow) and alkaline phosphatase (AP) and tryptic digest of ADP-ribosylated and RNAylated protein rS1. All samples were analysed by 12% SDS-PAGE. Left panel: Coomassie-stained gel, middle panel: autoradiography scan, right panel: merge of Coomassie stain and autoradiography scan.

FIG. 7: Characterisation of the specificity of ModB for NAD-RNA as substrate. a, Competition experiments using ³²P-NAD-RNA and an excess of unlabelled NAD revealed a preference of ModB for the former (compare FIG. 2c,d). ADPr-rS1 serves as a reference. b, Analysis of RNAylation dependency on the presence of a 5′-NAD-cap of the RNA. 10% SDS-PAGE analysis of in vitro RNAylation of the protein rS1 by ModB in the presence of either 5′-NAD− (NAD-³²P-Qβ), 5′-monophosphate- (5′-P³²-Qβ) or 5′-triphosphate-Qβ-RNA (5′-P³²PP-Qβ). c, Characterisation of ADPr-RNA as a substrate for ModB. As positive control, NAD-8mer was applied. All reactions were analysed by 12% SDS-PAGE. Left panel: Coomassie-stained gel, middle panel: autoradiography scan, right panel: merge of Coomassie stain and autoradiography scan.

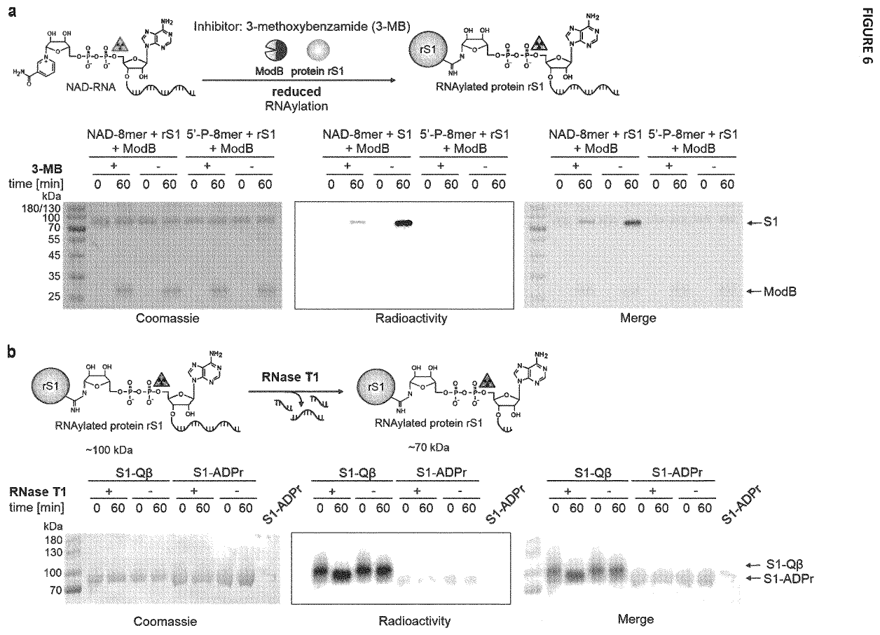

FIG. 8: Specific removal of the RNAylation using chemical and enzymatic treatments. a, Different ADP-ribose-protein linkages have been shown to be either stable or instable in the presence of HgCl₂ and neutral hydroxylamine, which represents a relatively straightforward and fast approach to identify ADP-ribosylation sites. Treatment with hydroxylamine hydrolyses linkages between glutamate and aspartate and ADP-ribose. HgCl₂ specifically cleaves thiol-glycosidic bonds. ADP-ribosylated and RNAylated protein rS1 were treated with hydroxylamine or HgCl₂. The removal of ADPr or RNA would result in a decrease of the radioactive signal of protein rS1. All samples were analysed by 12% SDS-PAGE. A decrease of the radioactive signal in comparison to the control (untreated) was not determined. b, in vitro kinetics of RNAylated protein rS1 in the presence of ARH1 or ARH3 analysed by 12% SDS-PAGE.

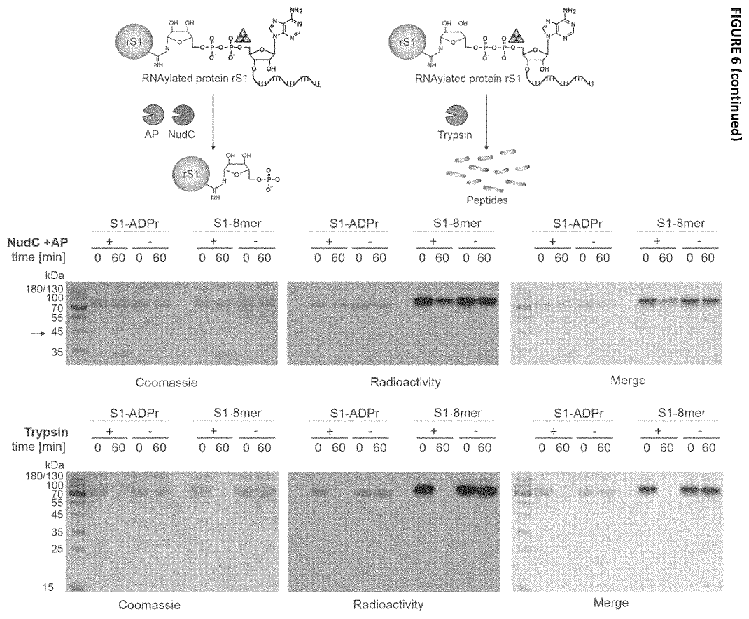

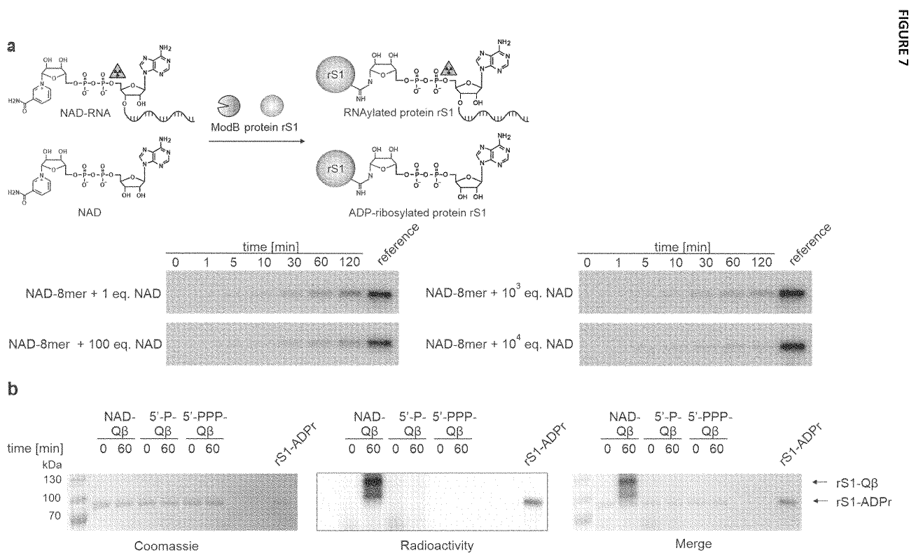

FIG. 9: LC-MS2 spectra of rS1 peptides harboring a peptide ribose-5-phosphate. LC-MS2 spectra of rS1 peptides harboring a peptide ribose-5-phosphate (R5P) modification at R139 and R426 from in vivo experiments. Sufficient peptide sequence coverage of manually validated spectra reveals solely arginine as the modified amino acid in vivo. Whilst ADPr escaped LC-MS detection, we identified ribose-5-phosphate (R5P), m/z=212.0086 Th, as a shorter fragment of ADPr reliably and unambiguously. R5P-linked arginine residues are boxed in yellow.

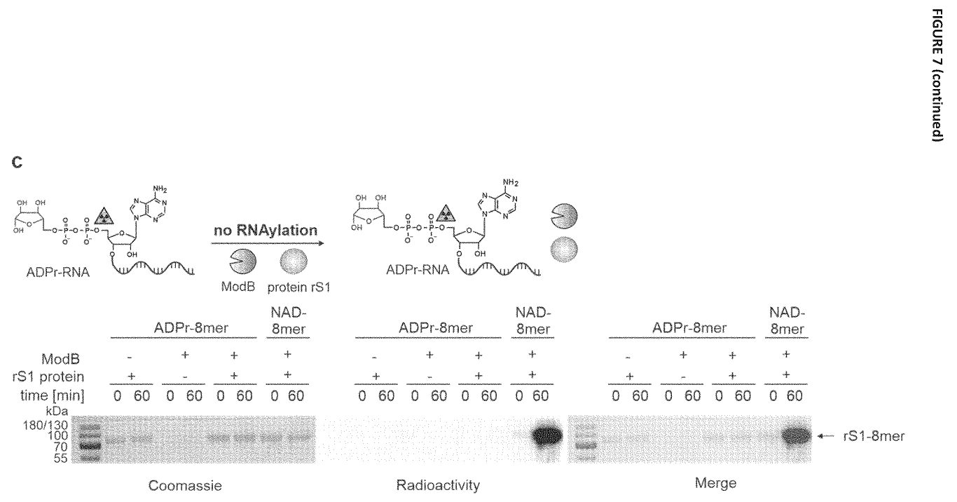

FIG. 10: Characterisation of ADP-ribosylation and RNAylation of R139 of rS1 in vivo. Peptide AFLPGSLVDVRPVRTHLEGK isolated from in vivo samples carries a R5P modification at R139 as indicated by a yellow box. Even though the peptide is longer due to a missed cleavage at R142, the peptide sequence and modification site was determined reliably. Peptide AFLPGSLVDVAPVRTHLEGK identified from an rS1 R139A or R139K mutant does not carry the R5P modification at position 139.

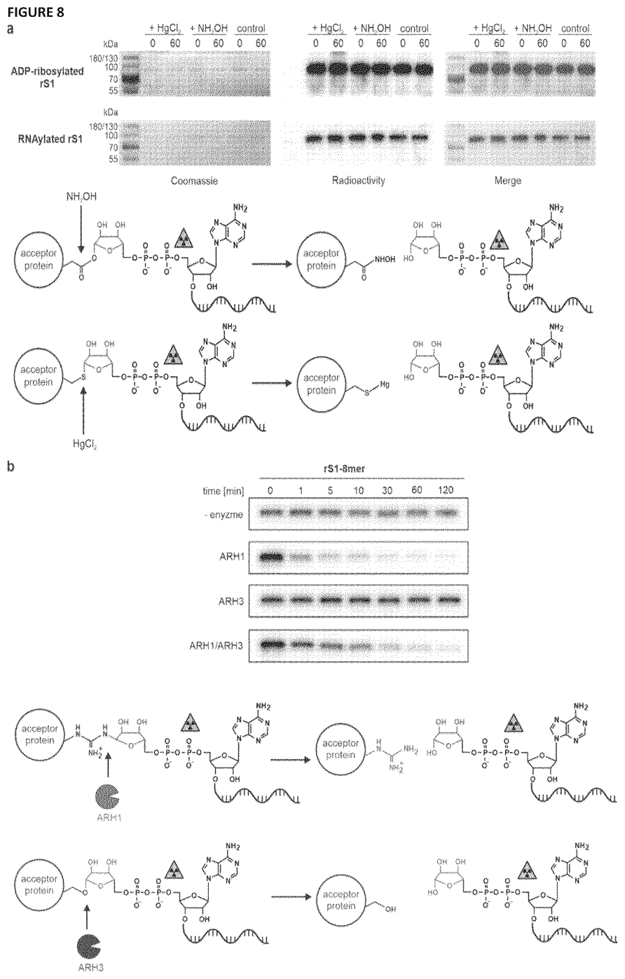

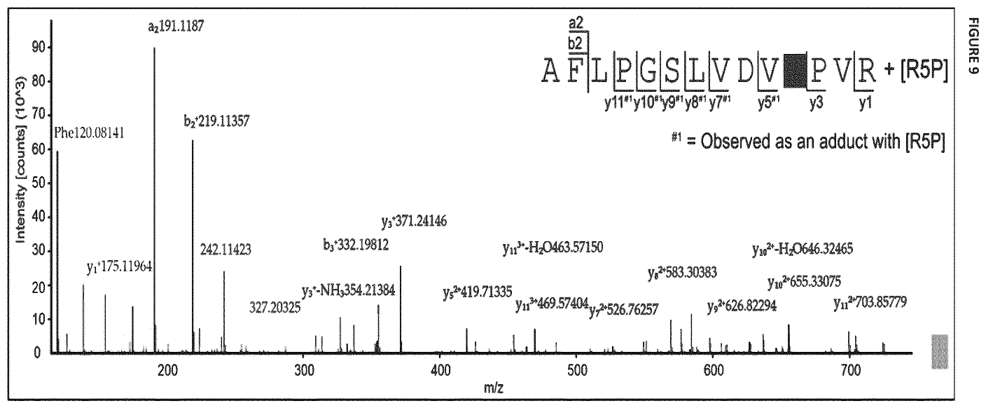

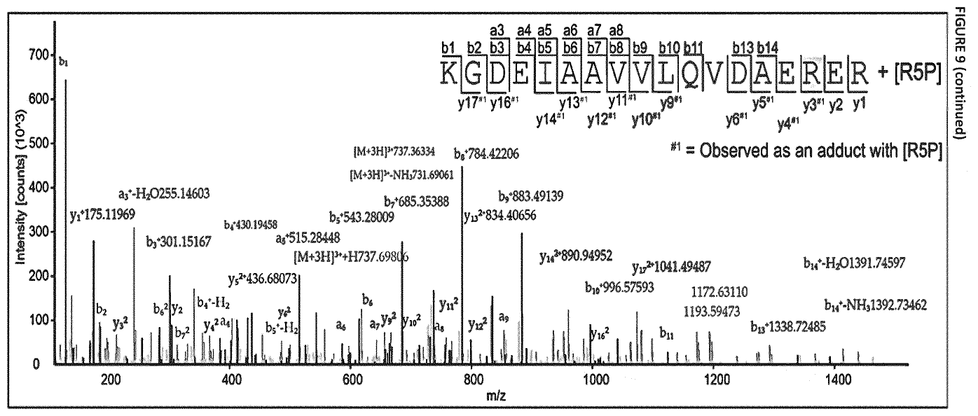

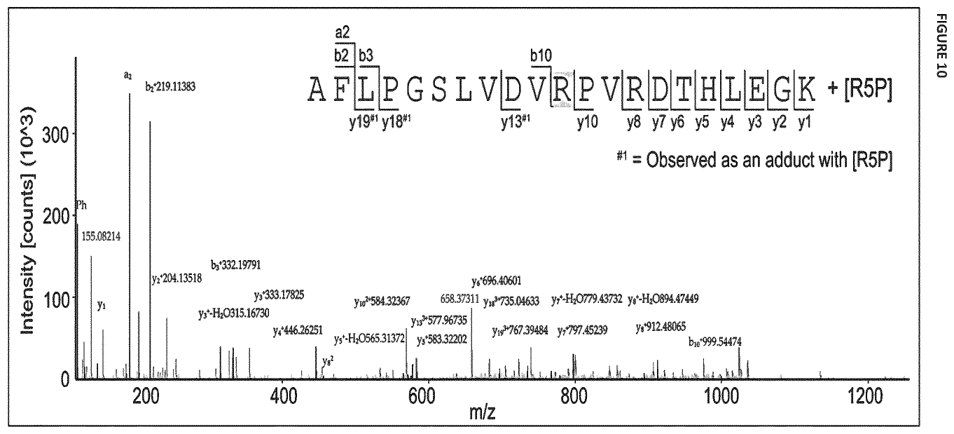

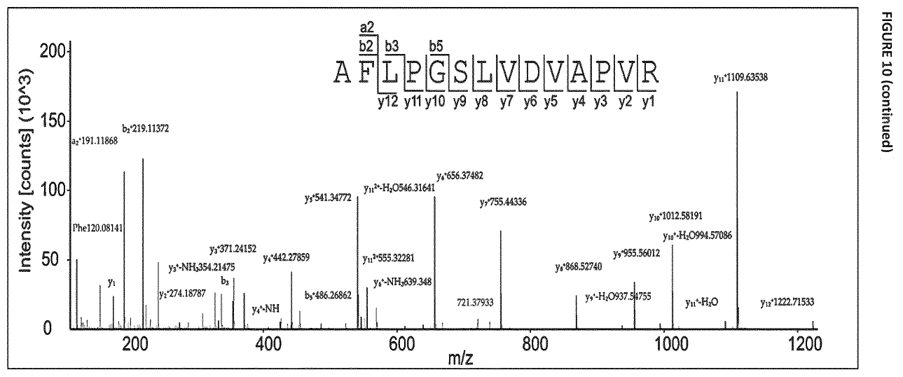

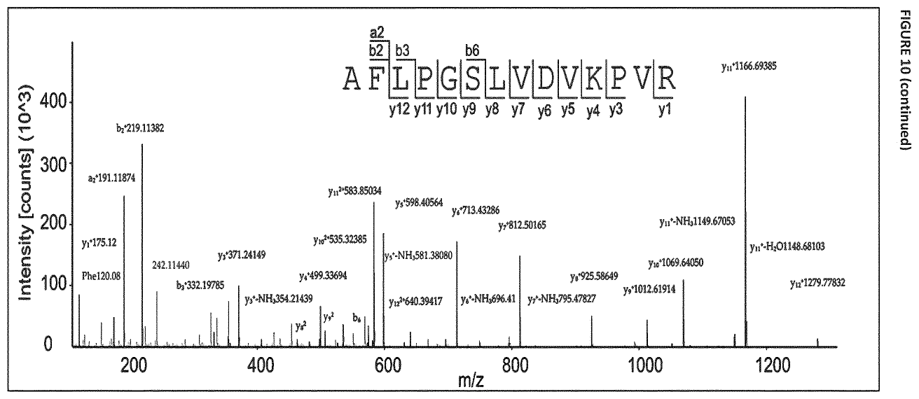

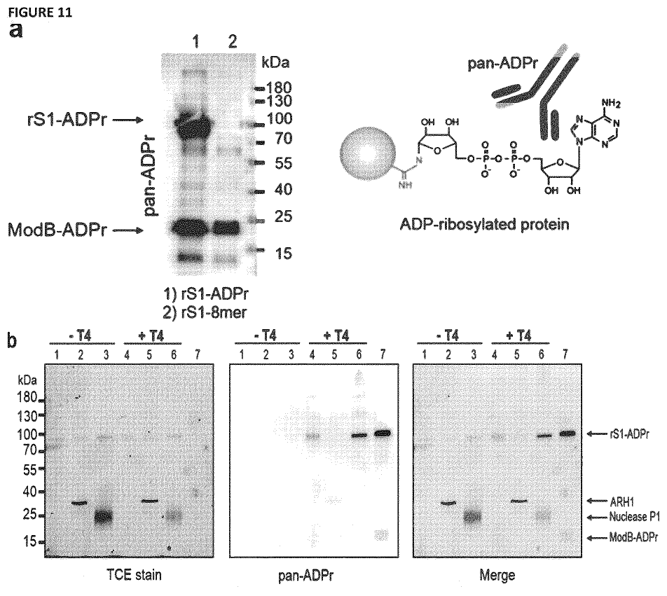

FIG. 11: In vivo characterisation of ADP-ribosylation and RNAylation by Western blot analysis. a, Analysis of the substrate specificity of the pan-ADPr antibody. In vitro prepared ADP-ribosylated or RNAylated protein rS1 was applied to evaluate the specificity of the antibody. b, Quantification of RNAylation using the combination of nuclease P1 digest and detection of protein-linked ADP-ribose by Western blot. Visualisation of protein load by TCE stain. Removal of the ADP-ribose signal by ARH1 treatment. The corresponding bar chart is shown in FIG. 4.

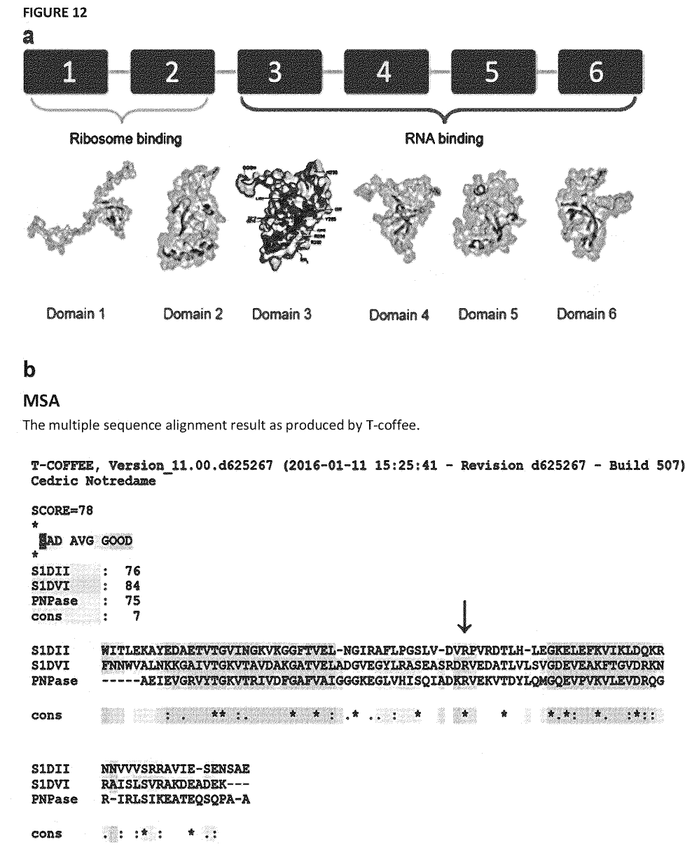

FIG. 12: ADP-ribosylation and RNAylation of rS1 domains D1-D6 and S1 motif of PNPase by ModB in vitro. a, Schematic illustration of the rS1-motifs of rS1, the crystal structures (PDB) of domains 1 (2MFI), 2 (2MFL), 4 (2KHI), 5 (5XQ5) and 6 (2KHJ) as well as an NMR structure of domain 3¹. b, Alignment of D2 and D6 of rS1 as well as of the S1 domain of PNPase using T-coffee expresso². R139 of D2 is highlighted with an arrow. c, ADP-ribosylation and d, RNAylation experiments were performed in triplicates and analysed by 16% Tricine SDS-PAGE, (L=Ladder). ModB and S1 domains are marked with black arrows. RNAylated rS1 domains, characterised by a significant shift compared to the non-modified proteins, are highlighted with red arrows. n=2 of biologically independent replicates. Reactions were performed using ³²P-NAD or ³²P-NAD-RNA 8-mer as a substrate for ModB.

FIG. 13: Characterisation of the influence of R139 of rS1 domain 2 on ADP-ribosylation and RNAylation. a, Analysis of the ADP-ribosylation of rS1 domain 2 and its mutants R139A and R139K by 16% Tricine-SDS-PAGE b, Quantification of relative intensities of ADP-ribosylation of rS1 domain 2 and its mutants R139A and R139K. n=2 of biologically independent replicates. c, Analysis of the RNAylation of rS1 domain 2 and its mutants R139A and R139K by 16% Tricine-SDS-PAGE. Inactive version of NudC V157A, E174A, E177A, E178A (NudC*) was used. d, Quantification of relative intensities of RNAylation of rS1 domain 2 and its mutants R139A and R139K. n=2 of biologically independent replicates.

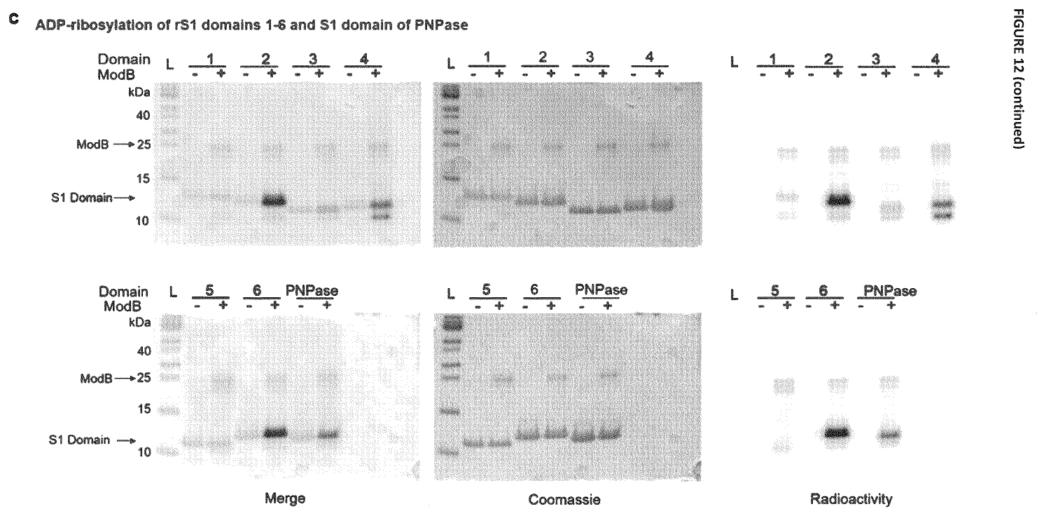

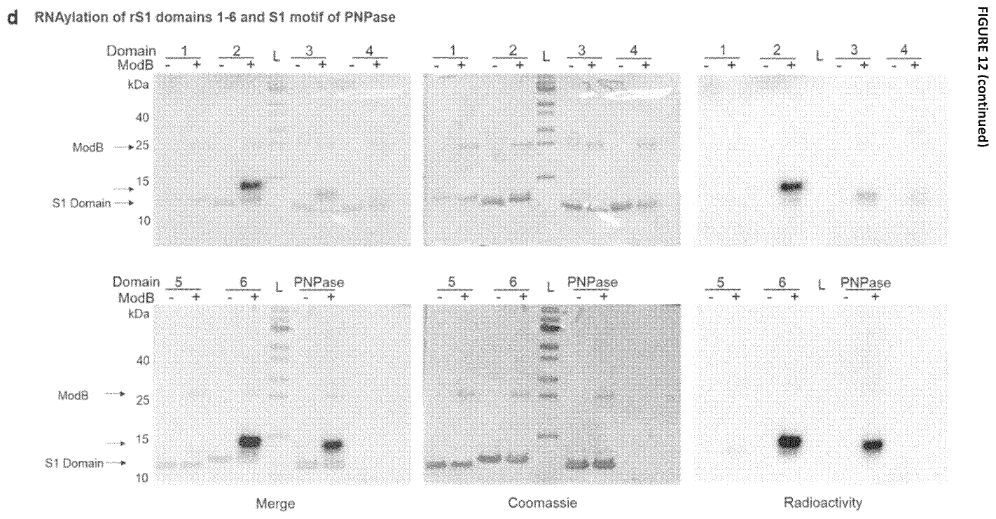

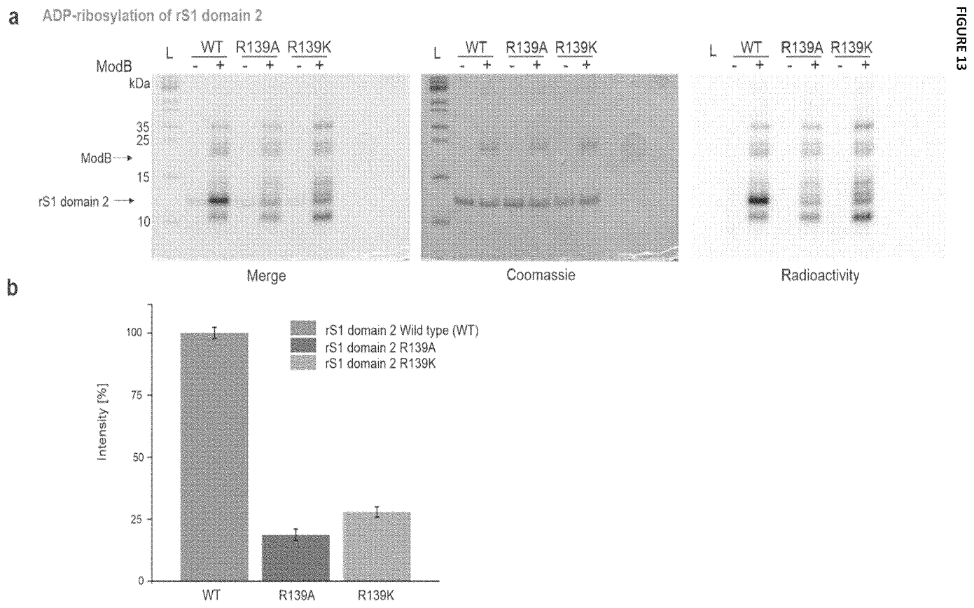

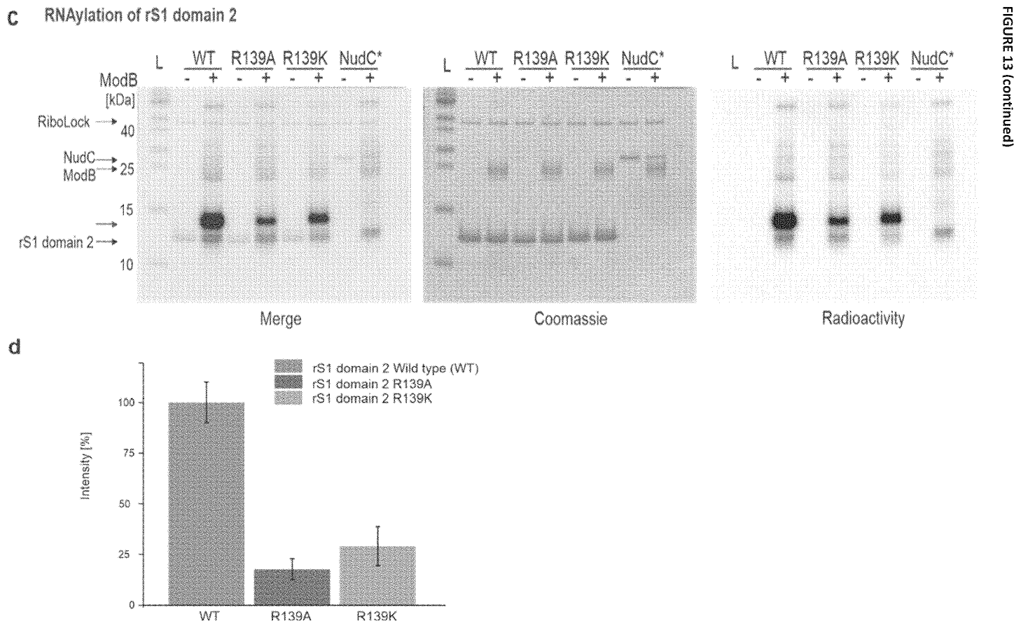

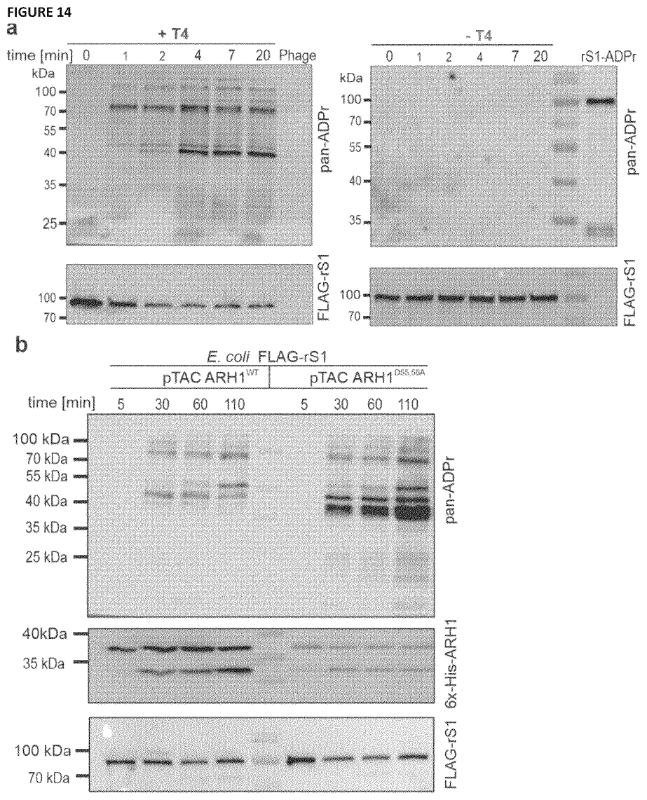

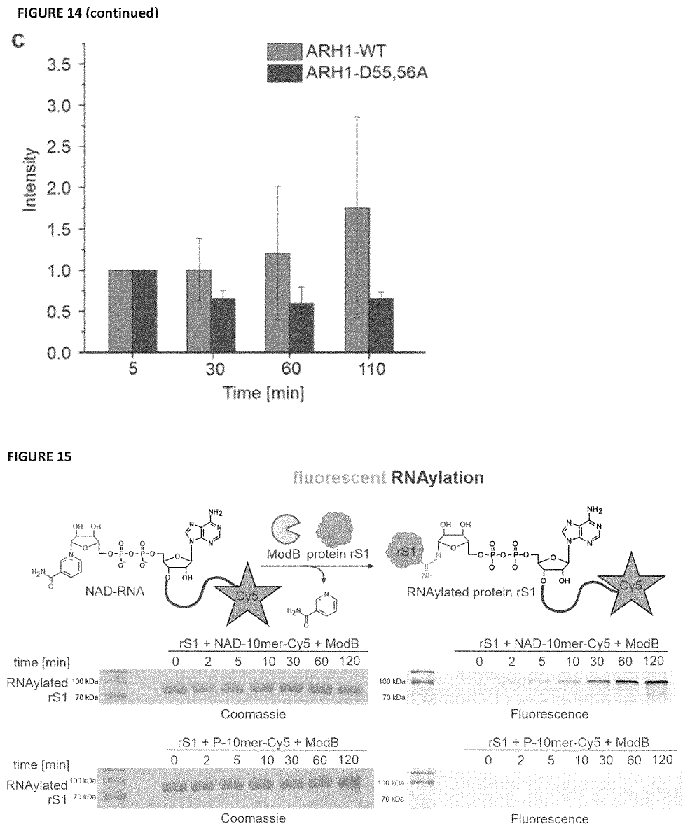

FIG. 14: in vivo characterisation of ADP-ribosylation and RNAylation. a, Time course of ADP-ribosylation of T4-infected (+T4) or non-infected (−T4) E. coli carrying a chromosomal fusion of the Flag-tag to rS1, analysed by Western Blot. In vitro prepared rS1-ADPr serves as positive control, n=4. b, Western blot to characterise the abundance of FLAG-rS1 during bacteriophage T4 infection in the presence of ARH1 WT and inactive ARH1 D55,56A. Comparable expression of ARH1 WT or ARH1 D55,56A was verified by Western blotting using a His-tag-specific antibody. Overexpression of ARH1 WT results in a significant decrease of the pan-ADPr signal. c, Quantification of FLAG-rS1 levels in T4 phage infected E. coli overexpressing ARH1 WT or inactive ARH1 D55,56A. FLAG-rS1 levels were determined by western blotting, shown in b.

FIG. 15: RNAylation of rS1 by ModB using a 3′ Cy5 labelled NAD-capped RNA in vitro. Enzyme kinetics of ModB were performed in the presence of a NAD-capped-10mer that has a 3′-Cy5 label. rS1 was used as a target for ModB that gets fluorescent (Cy5) upon RNAylation. Fluorescence signal was visualized using a Typhoon scanner. Samples were analyzed by 12% SDS-PAGE.

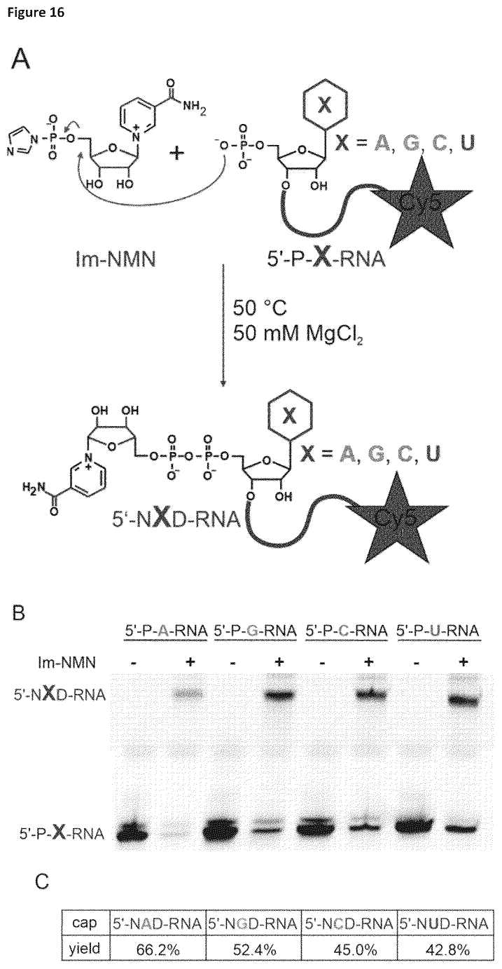

FIG. 16: Preparation of 5′P-X-10mer-Cy5 RNAs. A) 5′-P-X-RNAs were incubated in the presence of 1000-fold excess of Im-NMN, 50 mM MgCl2 for 5 hours at 50° C. 5′-NXD-RNAs were generated by coupling of Im-NMN to the 5′-monophosphate group as described by (23). B) Analysis of NXD-capping by analytical APB gel electrophoresis. 5′-P-X-RNAs served as negative controls (n=1). C) Comparison of the calculated yields of NXD-RNA capping reactions.

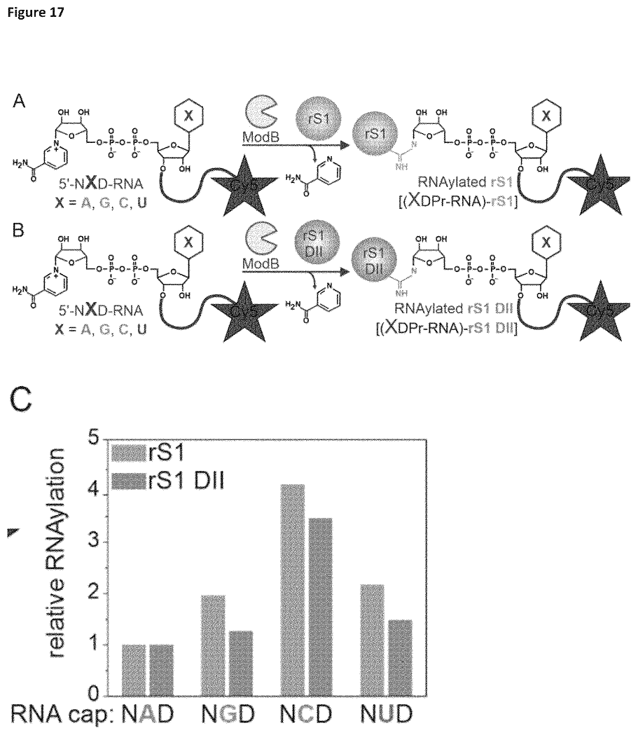

FIG. 17: RNAylation reactions of rS1 and rS1 DII by ModB in the presence of 5′-NXD-RNAs. A) The proposed RNAylation reaction mechanism for rS1 protein by ModB is shown. rS1 protein was incubated with each 5′-NXD-RNA in the presence of ModB to generate RNAylated rS1. B) RNAylation reaction of rS1 DII by ModB is depicted. In the presence of each 5′-NXD-RNA, the entire RNA chain was covalently linked to rS1 DII to generate RNAylated rS1 DII. C) Relative RNAylation efficiencies of rS1 and rS1 DII using different NXD-RNAs as a substrate for ModB.

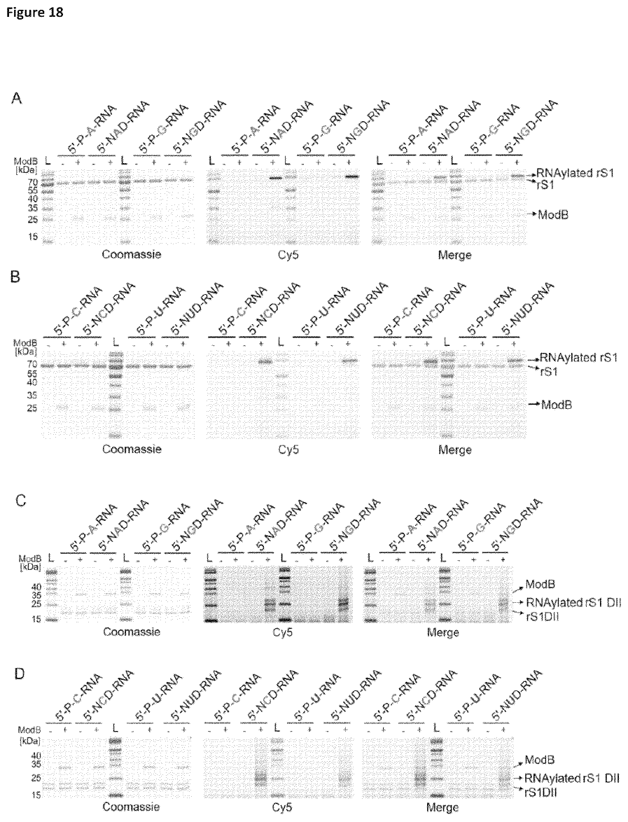

FIG. 18: In vitro RNAylation of rS1 and rS1 DII in the presence of differently capped RNAs by ModB. A,B) The RNAylation reactions of rS1 in the presence of 5′-P-X-10mer-Cy5 or 5′-NXD-10mer-Cy5 RNAs were analysed by 12% SDS-PAGE. RNAylated rS1 was detected as a shifted band in the presence of 5′-NXD-RNAs and ModB (n=2). C, D) rS1 DII RNAylation reactions with 5′-P-X-10mer-Cy5 or 5′-NXD-10mer-Cy5 RNAs were analysed by 15% Tricine gel electrophoresis. Shifted RNAylated rS1 DII was observed in the presence of 5′-NXD-RNAs and ModB (n=2).

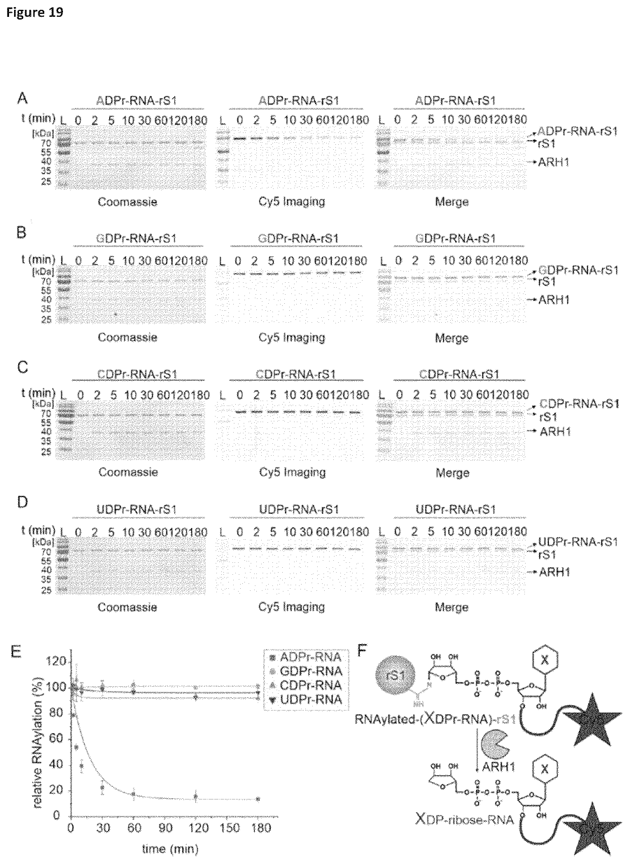

FIG. 19: In vitro ARH1 digestion kinetics of RNAylated rS1 with differently capped RNAs. RNAylated ADPr-RNA-rS1 (A), GDPr-RNA-rS1 (B), CDPr-RNA-rS1 (C) or UDPr-RNA-rS1 (D) proteins were subjected to ARH1 digestion for 0, 2, 5, 10, 30, 60, 120, and 180 min. Reactions were analysed by 12% SDS-PAGE. E) Calculated mean of relative RNAylation levels during ARH1 treatment. N=2 F) Schematic illustration of the mechanism of removing XDPr-RNA from RNAylated (XDPr-RNA)-rS1 by ARH1.

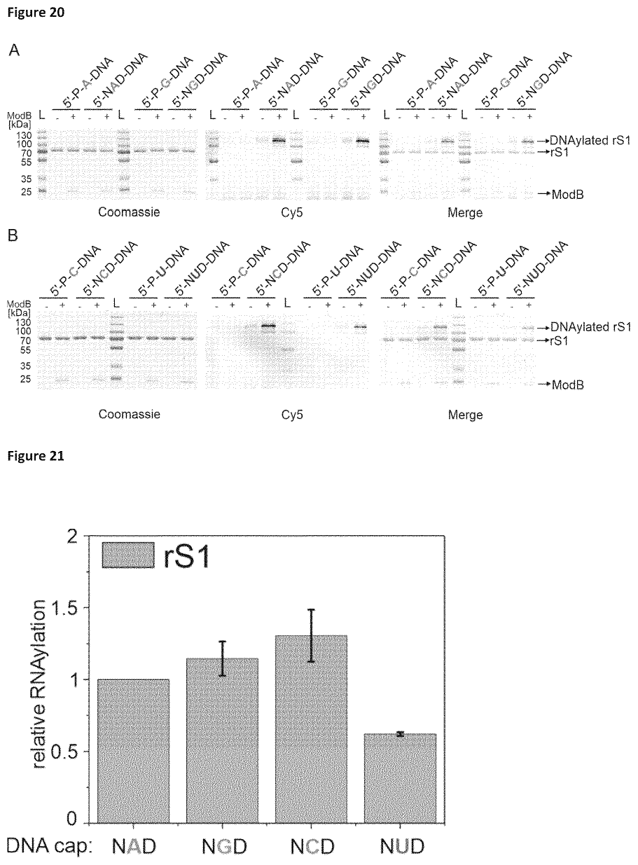

FIG. 20: In vitro RNAylation of rS1 and rS1 DII in the presence of differently capped DNAs by ModB. A,B) The RNAylation reactions of rS1 in the presence of 5′-P-X-10mer(DNA)-Cy5 or 5′-NXD-10mer(DNA)-Cy5 RNAs were analysed by 12% SDS-PAGE. RNAylated rS1 was detected as a shifted band in the presence of 5′-NXD-DNAs and ModB (n=2). (N=3).

FIG. 21: A) Relative RNAylation efficiencies of rS1 using different NXD-DNAs as a substrate for ModB. (N=3)

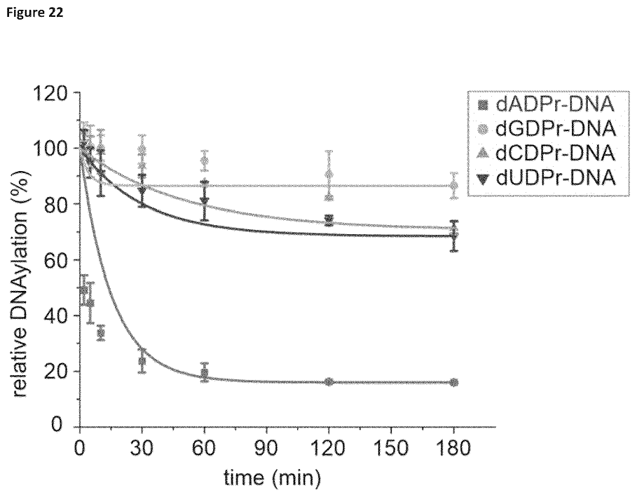

FIG. 22: In vitro ARH1 digestion kinetics of RNAylated rS1 with differently capped DNAs. RNAylated dADPr-DNA-rS1, dGDPr-DNA-rS1, dCDPr-DNA-rS1 or dUDPr-DNA-rS1 proteins were subjected to ARH1 digestion for 0, 2, 5, 10, 30, 60, 120, and 180 min. Reactions were analysed by 12% SDS-PAGE and the mean of relative RNAylation levels during ARH1 treatment calculated.

The Examples illustrate the invention:

### EXAMPLE 1—T4 ARTS CATALYSE RNAYLATIONS IN VITRO

To test the hypothesis that ARTs may accept NAD-RNAs as substrates, the three T4 ARTs were purified and incubated with a synthetic, site-specifically ³²P-labelled 5′-NAD-RNA 8mer to test for either self-modification or modification of target proteins. Modification is indicated by the acquisition of the ³²P-label by the ART or the target protein, respectively. While both Alt and ModA showed only a low extent of self- and target RNAylation (FIG. 5a), ModB rapidly RNAylated its known target, ribosomal protein S1 (rS1) without detectable self-RNAylation, as indicated by radioactive bands with the expected mobility in SDS-PAGE gels. In contrast, ADP-ribosylation in the presence of ³²P-NAD resulted in the modification of both proteins (ModB and rS1) with similar radioactive band intensities (FIG. 2a,b and FIG. 5b,c). The radioactive band did not appear when either ModB or rS1 were missing or when a 5′-³²P-monophosphate-RNA (5′-³²P-RNA) of the same sequence was used as a substrate for ModB (FIG. 5d).

### EXAMPLE 2—MODB PREFERS NAD-RNA OVER NAD

ModB-catalysed RNAylation of rS1 was strongly inhibited by the ART inhibitor 3-methoxybenzamide (3-MB) (FIG. 6a). The radioactive rS1 band did not disappear when the reaction product was treated with Rnase T1. This treatment would remove the ³²P-label if the RNA was non-covalently bound to rS1 or was covalently linked via other than 5′-terminal positions (FIG. 6b). The bacterial enzyme NudC²¹, which hydrolyses pyrophosphate bonds in various non-canonical cap structures, caused a decrease of the radioactive signal by 53% (FIG. 6c), indicating the generation of ribose 5′-phosphate modified rS1. The radioactive band, however, disappeared entirely upon treatment with trypsin (which digests rS1) (FIG. 6c). Collectively, these data strongly support the covalent linkage of a RNA to rS1 via a diphosphoriboside linkage as shown in FIG. 1b.

Competition experiments using ³²P-NAD-RNA and an excess of unlabelled NAD revealed a preference of ModB for the former, which is important for modification reactions in vivo, where NAD is much more abundant than NAD-RNA (FIG. 2c and FIG. 7a). ModB also accepted longer, biologically relevant RNAs with comparable activity (e.g., a Qβ3-RNA fragment of ˜100 nt²², FIG. 2d and FIG. 7b). RNAylation with this NAD-capped-Qβ-RNA caused protein rS1 (˜70 kDa) to move like a 100 kDa protein on an SDS-PAGE gel (FIG. 2e). Treatment of the RNAylated protein with nuclease P1, which hydrolyses 3′-5′ phosphodiester bonds but does not attack the pyrophosphate bond of the 5′-ADP-ribose, reverted this shift, and the radioactive product migrated like non-modified rS1 or ADPr-rS1 (FIG. 2e), again confirming the proposed nature of the covalent linkage.

To exclude the possibility that ModB might just remove the nicotinamide moiety from the NAD-RNA by hydrolysis, generating a highly reactive ribosyl moiety that could (via its masked aldehyde group) spontaneously react with nucleophiles in its vicinity²³, authentic ADP-ribose-modified RNA (site-specifically ³²P-labelled) were prepared and tested it as substrate. No radioactive band appeared (FIG. 7c), providing no support for spontaneous ADP-ribosylation.

### EXAMPLE 3—MODB MODIFIES SPECIFIC ARGININES IN RS1

To identify the amino acid residues in protein rS1 to which RNA chains are covalently linked during RNAylation, advantage was took of tools developed to analyse protein ADP-ribosylation. The radioactive signal of RNAylated protein rS1 (as prepared in FIG. 2b) did not change upon treatment with HgCl₂ (which cleaves S-glycosides resulting from Cys), NH₂OH (which hydrolyses O-glycosides) (FIG. 8a) and recombinant enzyme ARH3 (which hydrolyses O-ADPr glycosides specifically at serine residues) (FIG. 8b), while it was efficiently removed by treatment with human ARH1²⁴ (FIG. 3a-d). These findings indicate that the major product(s) of the ModB-catalysed RNAylation reaction are linked as N-glycosides via arginine residues (as shown in FIG. 3a,b).

To identify the amino acid residues which are targeted by ModB, in vitro modified rS1 was subjected to tryptic digest, chromatographic purification, and mass-spectrometric analysis. This LC/MS/MS analysis revealed three specific modification sites in rS1, namely R19, R139, and R426 (FIG. 9).

To establish the biological significance of RNAylation by T4 ARTs in vivo, (untagged) protein rS1 was isolated endogenous from non-infected and T4-infected E. coli, respectively. E. coli contains significant amounts of endogenous NAD-RNAs^{4,6}. Ribosomes were isolated, and rS1 was pulled down by poly-U-sepharose and subjected to LC/MS/MS analysis (FIG. 3e). This experiment confirmed the in vitro data and revealed the same three sites, namely R19, R139 and R426, at which phosphoribose modifications were abundant only in the T4-infected sample. (FIG. 3f). Site-directed mutagenesis further confirmed the modified residues: R139K and R139A mutants of protein rS1 were expressed in T4-infected E. coli, purified and analysed, revealing that these mutations abolish the modification (FIG. 10).

### EXAMPLE 4—DETECTION OF RNAYLATION IN VIVO

The mass spectrometric pipeline detected ADP-ribosylation and RNAylation in the same way, namely as ribose-5′-phosphate or ADPr fragment. To distinguish between the two modifications, an immunoblotting assay was considered with an antibody-like ADP-ribose binding reagent (“pan-ADPr”). The specificity of pan-ADPr was investigated by Western blotting with in vitro-prepared ADP-ribosylated or RNAylated proteins, respectively (FIG. 11a). As expected, rS1-ADPr and ModB-ADPr were both recognised by pan-ADPr and produced bands with high intensities, while no signal was observed for rS1-RNA, suggesting that pan-ADPr does not tolerate 3′-extensions of the ribose moiety. However, when rS1-RNA was digested with nuclease P1 prior to pan-ADPr treatment, thereby degrading the RNA and likely leaving rS1-ADPr, a strong signal, comparable to authentic rS1-ADPr, appeared in the blot (FIG. 4a).

This immunoblotting assay was applied to investigate ADP-ribosylation and RNAylation in vivo. A plasmid-borne copy of rS1 was applied in non-infected or T4-infected E. coli. Subsequently, rS1 was affinity-purified and its ADP-ribosylation analysed by pan-ADPr blotting (Data FIG. 11b). In agreement with our mass-spectrometric data, this experiment revealed extensive ADP-ribosylation of rS1 only in the T4-infected sample. After nuclease P1-treatment, the pan-ADPr signal intensity of the rS1 band increased significantly (FIG. 4b), indicating that ˜30% of the modified rS1 was RNAylated in vivo (measured as the difference between P1-treated and nuclease untreated sample). Moreover, the signal for ADP-ribose disappeared upon ARH1 treatment, again confirming the nature of the RNA-protein linkage (FIG. 11b).

### EXAMPLE 5—A RECOGNITION MOTIF FOR MODB

How ModB identifies its targets remains a puzzle. Target protein rS1 contains oligonucleotide-binding (OB) domains²². One structural variant of OB folds is the S1 domain, present in rS1 in six copies that vary in sequence (FIG. 12a,b). It was speculated that the S1 domain might be important for substrate recognition by ModB. To characterise ModB's specificity for different rS1 domains, each S1 domain of rS1 was individually cloned, expressed and purified and they were applied in an RNAylation assay (FIG. 4 c,d and FIG. 12c,d). For rS1 domains D2 and D6 high RNAylation signals were determined. In comparison, rS1 D1, D3, D4 and D5 domains were modified to a much lesser extent. Alignment of D2 and D6 of rS1, and the S1 domain of PNPase, another protein E. coli that possess an S1 domain, revealed that these S1 motifs share an arginine residue as part of the loop connecting strands 3 and 4 of the β-barrel²⁵ (FIG. 12b). This loop is packed on the top of the β-barrel, thereby likely accessible for ModB. For rS1 D2, this particular residue is R139, which was shown to be modified by mass spectrometry (FIG. 3f). Mutation analysis confirmed that the ADP-ribosylation level of D2 is dramatically reduced if R139 is substituted by alanine or lysine (FIG. 13). Based on these findings, it was screened for other E. coli proteins that harbour an S1 domain with an arginine in the loop between strands 3 and 4, and identified Rnase E. In our in vitro assays, Rnase E, which carries the S1 motif in its active site, was efficiently modified by ModB, while control proteins without S1 domain (BSA, NudC inactive quadruple mutant) were not, supporting the identification of the subgroup of S1 domains with an embedded arginine as the RNAylation target motif (FIG. 4e,f).

### EXAMPLE 6—MODIFICATION AND T4 REPLICATION CYCLE

rS1 is an important RNA-binding protein required for the translation of virtually all cellular mRNAs in E. coli. To investigate the biological consequences of rS1 modification by ModB, rS1 levels were analysed during T4 infection using an E. coli strain that contains a chromosomal fusion of rS1 with a FLAG-tag (FIG. 4g and FIG. 13a). Immediately after infection, rS1 levels dropped steeply, whereas they increased moderately over 20 min the absence of T4. It was thus speculated that ADP-ribosylation and/or RNAylation might influence the stability of rS1. To test this hypothesis, human ARH1 was overexpressed in E. coli during T4 infection, thought to remove ADP-ribose and linked RNA. As a control, a largely inactive ARH1 D55, 56A mutant was overexpressed. Indeed, with active ARH1, the ADP-ribosylation signal was dramatically reduced (FIG. 14b), while the mutant showed a pattern similar to the parent strain (FIG. 14a). Using these constructed E. coli strains, the influence of ADP-ribosylation and RNAylation on rS1 levels was analysed during phage infection. Indeed, the strain expressing active ARH1 showed an increase in rS1 levels over time, like the uninfected sample, whereas the mutant strain exhibited declining levels, like the T4-infected sample without ARH1 (FIG. 14b,c). Thus, the removal of ADPr and RNA chains during phage infection coincides with a stabilisation of the rS1 level.

To investigate if these modifications are important for the lysogenic behaviour of the phage, E. coli strains expressing either ARH1 or its inactive mutant with T4 were infected and monitored the optical density over time (FIG. 4i). In the inactive mutant strain, bacterial lysis started 50 min post-infection, while delayed lysis (120 min) was observed when active ARH1 was overexpressed (FIG. 4i). Collectively, these data indicate that ADP-ribosylation and/or RNAylation interfere with protein stability and modulate the course and efficiency of T4 infection.

### EXAMPLE 7—RNAYLATION OF PROTEINS USING NND (=NXD)-CAPPED RNAS AND DNAS

This example shows that ModB accepts 5′-NGD-, NCD-, or NUD-capped-RNAs, in addition to 5′-NAD-RNA, as a substrate for an RNAylation reaction. The exchange of the RNA-cap, from NAD to NGD, NCD or NUD, does not change the catalytic activity of ModB. This finding indicates that the catalytic pocket of ModB does not sense the adenosine moiety of NAD. In contrast, nicotinamide moiety might be crucial for substrate recognition by ModB. Furthermore, applying NGD-, NCD- or NUD-RNA caps, which are not naturally occurring, will enable a flexible and applicable design of 5′-NXD-RNA as a substrate for RNAylation reactions. Therefore, target proteins of ModB can be RNAylated by any preferred RNA sequence. Finally, this example shows that GDPr-, UDPr-, and CDPr-linked RNAs are not removed by the humane ADP-ribose hydrolase ARH1 thereby showing an increase in RNA-protein stability. These properties set the foundation to generate novel in vitro RNA-protein conjugates that can be applied to eukaryotic systems in vivo in the future.

### 7.1 Imidazolide Reaction LED to an Efficient 5′-NXD-Capping of all Monophosphorylated RNAs

In comparison to NAD-RNAs, which have been described in all kingdoms of life, NUD-RNA, NCD-RNA as well as NGD-RNA are not described in biological systems yet. Thus, to verify if NXD-capped RNAs can be applied as a substrate for RNAylation, they were generated via chemical synthesis. Here, 5′-NXD-capping of 5′-monophosphorylated-RNAs was achieved using imidazolide reaction by coupling Im-NMN to the 5′-monophosphate group of an RNA (FIG. 16A). The reaction products were characterised by APB gel electrophoresis to investigate the capping reaction efficiency (FIG. 16B). Calculated 5′-NXD-RNA yields showed that the capping efficiency of the reaction was ranging between 42.8%, corresponding to NUD capping, and 66.2% observed for NAD capping. For NGD and NCD 52.4% and 45.0% capping was calculated, respectively (FIG. 16C). Capping efficiencies were in agreement with previous reports.

### 7.2 ModB Accepts 5′-NXD-Capped RNAs as a Substrate for RNAylation Reaction

The successful preparation of NXD-capped RNAs allowed to examine the substrate scope of ModB. It was hypothesised that all tested 5′-NXD-capped-RNAs can be accepted by ModB for an RNAylation reaction.

FIGS. 17A and 17B show the proposed mechanisms of rS1 and rS1 DII RNAylation reactions. In the presence of 5′-NXD-10mer-Cy5 RNAs, ModB might covalently link the entire RNA chain to the target protein by an RNAylation reaction.

To verify if NXD-capped RNAs can be applied as a novel substrate for ModB, in vitro RNAylation reactions were performed (FIG. 17C and FIG. 18). RNAylation reactions were performed with 5′-P-X-RNAs (negative controls) and 5′-NXD-RNAs in the presence and the absence of ModB. NAD-RNA served as a positive control and reference for RNAylation. The data show that RNAylation of rS1 protein by ModB was achieved irrespective of the second nucleotide positioned in the cap structure. It was identified that NGD-RNA, NCD-RNA or NUD-RNA are accepted as a novel substrate for RNAylation by ModB. Moreover, the RNAylation of rS1 with NXD-RNAs alters the protein size, which causes a change in the running behaviour of the modified protein in comparison to the non-modified protein (FIGS. 18A and 18B).

The calculated RNAylation yield of rS1 in the presence of 5′-NGD-RNA or 5′-NUD-RNA was similar to the RNAylation with 5′-NAD-RNA. Surprisingly, RNAylation reaction with 5′-NCD-RNA resulted in a four times higher yield than 5′-NAD-RNA (FIG. 3C). No RNAylation was detected in the absence of ModB, proving that the rS1 does not covalently attach an RNA in a non-enzymatic way. Additionally, 5′-P-X-RNA was not accepted as a substrate by ModB, and RNAylation reactions have taken place only in the presence of 5′-NXD-capped RNAs (FIGS. 18A and 18B).

It can be shown that rS1 can be RNAylated in the presence of 5′-NXD-RNAs by ModB. In addition, it was asked the question of whether different target proteins can be RNAylated by ModB using NXD-RNAs as substrate. Thus, the RNAylation of another target protein, rS1 DII, was characterised in the presence of 5′-NXD-RNAs by ModB. In contrast to the already investigated rS1 (68 kDa), rS1 DII is a small protein with a molecular weight of 9.7 kDa.

Similarly to rS1 protein, it can be shown that rS1 DII was RNAylated in the presence of 5′-NXD-RNAs by ModB. Moreover, a distinct size shift of the RNAylated protein can be observed (FIGS. 18C and 18D). The calculated RNAylation efficiencies reveal the same trend as described for rS1. Again, the highest RNAylation of rS1 DII was observed in the presence of NCD-RNA (FIG. 17C).

Thus, the data show that both rS1 and rS1 DII were successfully RNAylated in the presence of 5′-NXD-RNAs and ModB. Furthermore, RNAylation efficiency did not differ between target proteins, meaning that various target proteins can be RNAylated with the same efficiency irrespective of their molecular weight.

### 7.3 ARH1 Specifically Hydrolyses N-Glycosidic Linkages of ADP-Ribosyl-Arginine Residues

In eukaryotic systems, ARH1 is the major player in removing ADP-ribosylations. Thus, the stability of in vitro prepared RNAylated protein conjugates that are applied to eukaryotic systems depends on the enzymatic activity of ARH1. It was speculated that the exchange of the covalent attached ADP-ribose-RNA to GDPr-RNA, CDPr-RNA or UDPr-RNA changes the substrate recognition by ARH1.

To test whether the covalently linked XDPr-RNA is removed by ARH1, rS1 protein RNAylated with 5′-NXD-RNAs were digested with ARH1 in vitro (FIG. 19). rS1 protein RNAylated with 5′-NAD-RNA (ADPr-RNA-rS1), served as a positive control for ARH1 digestion. It can be shown that the ARH1 efficiently removes the ADPr-RNA from ADPr-RNA-rS1. Relative RNAylation levels decreased to 20% after 30 min of ARH1 treatment (FIG. 19A,E). In contrast, ARH1 could not remove GDPr-RNA, CDPr-RNA or UDPr-RNA from RNAylated GDPr-RNA-rS1, CDPr-RNA-rS1 or UDPr-RNA-rS1 proteins (FIG. 19B-E). Compared to the hydrolysis of RNAylation in the presence of ADPr-RNA, the reaction takes place 40 times and 20 times slower in the presence of CDPr-RNA- and UDPr-RNA, respectively. Thus, ARH1 specifically hydrolyzes the N-glycosidic linkage of ADP-ribosyl-arginine residues.

### 7.4 RNAylation of Proteins Using NND (=NXD)-Capped DNAs

The in n vitro RNAylation of rS1 and rS1 DII in the presence of differently capped DNAs by ModB is son in FIG. 20. The RNAylation reactions of rS1 in the presence of 5′-P-X-10mer(DNA)-Cy5 or 5′-NXD-10mer(DNA)-Cy5 RNAs were analysed by 12% SDS-PAGE. RNAylated rS1 was detected as a shifted band in the presence of 5′-NXD-DNAs and ModB (n=2).

In addition, the relative RNAylation efficiencies of rS1 using different NXD-DNAs as a substrate for ModB was etsed (FIG. 21).

Finally, the in vitro ARH1 digestion kinetics of RNAylated rS1 with differently capped DNAs were analysed (FIG. 22). For this analysis RNAylated dADPr-DNA-rS1, dGDPr-DNA-rS1, dCDPr-DNA-rS1 or dUDPr-DNA-rS1 proteins were subjected to ARH1 digestion for 0, 2, 5, 10, 30, 60, 120, and 180 min and the reactions were analysed.

### EXAMPLE 8—DISCUSSION

### 8.1 Discussion of Examples 1 to 7

To date, all interactions between RNAs and proteins are described to be based on non-covalent interactions²⁶. In contrast, it is show herein that ADP-ribosyltransferases can attach NND-capped RNAs to target proteins in a covalent fashion. This finding represents a distinct biological function of the NND-cap on RNAs in bacteria, namely activation of the RNA for enzymatic transfer to an acceptor protein. RNAylation of target proteins was discovered, which is a novel post-translational protein modification, playing a role in the infection of the bacterium E. coli by bacteriophage T4. Our data indicate that T4 ART ModB modifies proteins that possess an S1 RNA binding domain. Specific arginine residues to be modified were identified, thereby increasing molecular weight and negative charge of the target protein and undoubtedly causing major changes of the properties and functions of the modified proteins. The post-translational modification of crucial players in bacterial translation and transcription demonstrates the importance of the known ADP-ribosylation and the newly discovered RNAylation reaction for bacteriophage pathogenicity. Introduction of the human ADP ribosylhydrolase ARH1, which removes these modifications, into E. coli, caused a significant delay in bacterial lysis upon phage infection.

The reason why ARTs attach RNAs to proteins involved in translation may be that these RNAs help (e.g., by base pairing) to preferentially recruit mRNAs encoding for phage proteins to the ribosomes and thereby guarantee their biosynthesis. Likewise, the observation that Rnase E, the major player in RNA turnover in E. coli, is RNAylated at its catalytic centre by ModB may suggest that the T4 phage, after reprogramming transcription by Alt and ModA, shuts down RNA degradation in the host to ensure a long half-life of phage mRNAs. We are working vigorously on methods for identifying the RNAs attached to target proteins, which will allow the elucidation of their biochemical mechanisms.

ARTs are known to occur not only in bacteriophages, and ADP-ribosylated proteins have been detected in hosts upon infections by various viruses, including influenza, corona, and HIV. In addition to viruses using ARTs as weapons, the mammalian antiviral defence system applies host ARTs to inactivate viral proteins. Moreover, mammalian ARTs and poly-(ADP-ribose) polymerases (PARPs) are regulators of critical cellular pathways and are known to interact with RNA²⁷. Thus, ARTs in different organisms might catalyse RNAylation reactions, and RNAylation can be expected as a phenomenon of broad biological relevance.

Finally, RNAylation may be considered as both a post-translational protein modification and a post-transcriptional RNA modification. Our findings challenge the established views of how RNAs and proteins can interact with each other. The discovery of these new RNA-protein conjugates comes at a time when the structural and functional boundaries between the different classes of biopolymers become increasingly blurry^{28,29}.

### 8.2 Further Discussion of Example 7

In contrast to the recently identified NAD-RNAs, NGD-, NCD-, or NUD-RNAs have not been discovered in biological systems yet. Therefore, 5′-NXD-capped-RNAs were generated by chemical synthesis using imidazolide reaction. In addition to earlier studies, it is shown herein that synthetic 3′-Cy5 labelled RNAs can be used as a template for imidazolide reaction to prepare fluorescent NXD-capped-RNA/DNA. The calculated capping efficiencies for 5′-NXD-capped-RNAs were similar to previous reports. Furthermore, the generated 5′-NXD-RNAs were used to investigate the substrate specificity of ModB. In vitro RNAylation reactions of rS1 and rS1 DII by ModB were performed in the presence of 5′-NXD-RNAs.

It was discovered that 5′-NXD-capped-RNAs/DNAs are accepted as a substrate by ModB. Hence, RNAylation reaction takes place irrespective of the first base of RNA. This means that A can be exchanged to G, C, or U in the cap structure, and the capped-RNA can be used as a substrate for RNAylation reaction by ModB as well.

To date, a protein crystal structure of ModB and its substrate NAD are not available. For this reason, the substrate specificity of ModB remains elusive. The exchange of the RNA-cap, from NAD to NGD, NCD or NUD, does not change the catalytic activity of ModB. This finding indicates that the catalytic pocket of ModB does not sense the adenosine moiety of NAD. In contrast, nicotinamide moiety might be crucial for substrate recognition by ModB. Thus, it is conclude that the only essential requirement of the RNAylation substrate design is the NMN moiety of the NAD-RNA-cap.

Moreover, the data herein show that 5′-NGD-RNA and 5′-NUD-RNA resulted in a similar RNAylation yield as 5′-NAD-RNA, which was used as a reference. Interestingly, an increase in the RNAylation efficiency of ModB was identified in the presence of 5′-NCD-RNA.

Recently, it was shown that the naturally occurring RNAylation affects the molecular properties of target proteins, such as the molecular weight (Höfer et al. (2021), bioRxiv, 2021.2006.2004.446905). Example 7 shows that the covalent attachment of an NGD-RNA, NCD-RNA or NUD-RNA to the target proteins rS1 and rS1 DII increases the protein size. In conclusion, discovering NXD-RNAs as novel substrates for ModB, enables a flexible design of RNA-oligos applied in an RNAylation reaction. RNAylation substrates can be generated by solid-phase synthesis or in vitro transcription. Especially in vitro transcription reaction allows for the preparation of biological relevant transcripts longer than 80 nucleotides. Here, G-initiation results typically in high transcription yields, which are needed to prepare RNAylation substrates such NGD-RNAs. Moreover, our data show that higher RNAylation yields can be achieved by using 5′-NCD-RNA as a substrate.

Furthermore, the stability of XDPr-RNA-proteins in the presence of the human ARH1 was studied in Example 7. ARH1 is the only known eukaryotic enzyme yet to remove RNAylation from a target protein in vivo. The catalytic activity of ARH1 in the presence of differentially capped RNAs has not been tested before. Example 7 shows that ARH1 is not capable of efficiently removing RNAylation in the presence of GDPr-RNA, UDPr-RNA, CDPr-RNA. The in vitro kinetic data demonstrate that ARH1 strongly prefers arginine linked ADPr-RNA over GDPr-RNA, UDPr-RNA, CDPr-RNA as a substrate.

In conclusion, applying NXD-RNAs as substrates for the RNAylation of proteins improves the understanding of the substrate specificity of ModB and ARH1. While ModB accepts all four different NXD-RNA derivates as substrates, ARH1 is highly specific for the hydrolysis of the N-glycosidic linkage of ADP-ribosyl-arginine. Thereby, GDPr-RNA-rS1, UDPr-RNA-rS1, CDPr-RNA-rS1 proteins have increased stability in the presence of human ARH1 in vitro. These properties set the foundation to generate in vitro RNA-protein conjugates that can be applied to eukaryotic systems in vivo in the future.

### EXAMPLE 9—EXTENDED DATA

Extended Data Table 1: RNAs used in this study

| RNA | RNA sequence |
|---|---|
| 8-mer | 5′ P-ACAGUAUU |
| 10-mer | 5′P-AGACUUCGAC |
| Qβ (100-mer) | AUCUUGAUACUACCUUUAGUUCGUUUAAACACGUUCUUGAUAG |
| | UAUCUUUUUAUUAACCCAACGCGUAAAGCGUUGAAACUUUGGG |
| | UCAAUUUGAUCAUG |
| 5′P-A-10 mer-Cy5 | 5′P-AGACUUCGAC-Cy5 |
| 5′P-G-10 mer-Cy5 | 5′P-GGCAUUCGAC-Cy5 |
| 5′P-C-10 mer-Cy5 | 5′P-CGCAUUCGAC-Cy5 |
| 5′P-U-10 mer-Cy5 | 5′P-UGCAUUCGAC-Cy5 |

Extended Data Tables 2: Genomic DNA sequence of ARTs, rS1 variants and ADP-ribose hydrolases. Start codon in italic; thrombin cleavage site in bold; mutations in red and bold; restriction sites underlined

| Gene [5′, 3′ | |
|---|---|
| restriction | |
| site] | DNA sequence |
| Alt [NcoI, | CCATGGGAGAACTTATTACAGAATTATTTGACGAAGATACTACTCTTCCAA |
| XhoI] | TTACAAACTTATATCCAAAGAAGAAAATACCGCAAATTTTTTCAGTTCATGT |
| | TGATGATGCAATTGAACAACCAGGCTTTCGTTTATGTACCTATACATCTGG |
| | AGGTGATACTAATCGTGATTTAAAGATGGGCGATAAAATGATGCATATTGT |
| | TCCTTTTACATTAACTGCTAAAGGTTCAATTGCTAAATTAAAAGGTCTTGGT |
| | CCAAGCCCAATTAATTATATCAATTCAGTTTTTACTGTTGCAATGCAAACAA |
| | TGCGCCAGTATAAAATTGATGCCTGTATGCTCCGTATTCTTAAGTCTAAAA |
| | CTGCTGGCCAAGCTCGACAAATTCAAGTTATTGCTGATAGACTTATCCGTA |
| | GTCGTTCAGGTGGTAGATACGTCCTTCTTAAGGAACTCTGGGATTACGAT |
| | AAAAAGTATGCATATATTCTTATACATCGCAAAAATGTATCACTAGAAGACA |
| | TTCCAGGAGTTCCGGAAATTAGTACCGAGCTCTTTACTAAAGTTGAATCGA |
| | AGGTCGGTGATGTTTATATCAATAAAGATACTGGGGCTCAAGTAACTAAAA |
| | ATGAGGCAATTGCAGCATCTATTGCGCAAGAAAATGATAAACGTTCTGAC |
| | CAAGCTGTAATCGTTAAAGTTAAAATTTCCCGTAGAGCAATTGCGCAAAGT |
| | CAGTCATTGGAATCTTCTAGATTTGAAACACCAATGTTTCAAAAATTTGAG |
| | GCTTCAGCGGCCGAATTAAATAAACCAGCGGACGCGCCTTTAATTTCTGA |
| | TTCTAATGAATTAACGGTAATTTCTACTTCAGGATTTGCACTAGAGAATGCT |
| | CTTAGCAGTGTTACAGCTGGGATGGCATTCAGAGAAGCTTCTATAATTCCT |
| | GAAGATAAAGAATCCATTATTAACGCAGAAATAAAAAATAAAGCTTTAGAA |
| | AGATTACGAAAAGAATCTATTACTTCAATAAAAACCTTAGAAACTATTGCTT |
| | CTATCGTCGATGATACTTTAGAAAAATATAAGGGTGCTTGGTTTGAAAGAA |
| | ATATTAACAAACATTCGCATTTAAACCAAGATGCTGCAAATGAGTTAGTAC |
| | AAAATTCTTGGAATGCAATAAAAACAAAGATTATTCGAAGAGAATTACGTG |
| | GATATGCTCTTACCGCTGGATGGTCATTACATCCTATAGTCGAAAATAAAG |
| | ATTCATCTAAATACACACCAGCGCAAAAACGCGGAATTCGTGAATACGTA |
| | GGTTCAGGATATGTAGACATAAATAATGCTCTTTTGGGATTATATAATCCA |
| | GATGAGCGTACAAGTATTTTGACAGCATCTGACATAGAAAAAGCTATTGAT |
| | AATTTAGATTCAGCCTTTAAAAATGGTGAACGATTACCAAAAGGTATTACTT |
| | TGTATCGTTCACAACGAATGTTACCTTCAATATACGAAGCAATGGTAAAAA |
| | ATCGAGTTTTTTATTTTAGAAACTTTGTGTCAACATCATTATATCCAAATATT |
| | TTTGGTACTTGGATGACTGATTCATCTATAGGTGTTTTACCAGACGAAAAG |
| | CGTTTAAGCGTTTCTATTGATAAAACTGATGAAGGACTTGTAAATTCTAGC |
| | GATAATTTAGTTGGAATTGGATGGGTTATTACTGGGGCTGATAAGGTCAAT |
| | GTTGTTTTACCCGGTGGAAGTTTAGCGCCTTCAAATGAAATGGAAGTCATT |
| | TTGCCACGTGGATTAATGGTCAAAGTTAATAAAATAACCGATGCATCTTAC |
| | AATGATGGAACAGTTAAAACTAACAACAAGCTTATTCAAGCTGAAGTTATG |
| | ACCACAGAAGAACTCACCGAATCGGTAATCTATGACGGAGACCATTTAAT |
| | GGAAACTGGTGAATTGGTTACAATGACAGGTGATATAGAAGATAGAGTTG |
| | ACTTTGCATCATTTGTTTCATCAAATGTTAAACAGAAAGTAGAATCATCTCT |
| | TGGAATTATTGCGTCTTGCATAGATATTGCAAACATGCCTTACAAGTTCGT |
| | TCAAGGACTGGTGCCGCGCGGCAGCCTCGAG |
| ModA | CCATGGGAAAATACTCAGTAATGCAACTAAAAGATTTTAAAATAAAATCAAT |
| [NcoI, | GGATGCATCGGTGCGTGCTTCTATTCGTGAAGAATTACTTTCTGAAGGGT |
| XhoI] | TTAATTTATCTGAAATTGAACTTTTAATTCATTGTATTACTAATAAACCAGAT |
| | GACCATTCTTGGTTAAATGAAATAATCAAATCTCGTTTGGTTCCAAACGAT |
| | AAACCTCTTTGGAGAGGTGTTCCAGCTGAGACTAAACAAGTATTAAATCAA |
| | GGAATTGATATTATTACATTTGATAAAGTCGTATCAGCTTCATATGATAAAA |
| | ATATAGCTCTACATTTTGCTTCTGGTTTAGAGTATAACACACAAGTTATTTT |
| | TGAATTCAAAGCTCCTATGGTATTCAATTTCCAGGAGTATGCTATAAAAGC |
| | TCTACGCTGTAAAGAATACAATCCAAACTTTAAGTTTCCGGATAGTCATCG |
| | TTATCGTAATATGGAATTAGTTTCAGATGAACAAGAAGTAATGATACCAGC |
| | TGGAAGTGTATTTAGAATTGCAGATAGATATGAGTATAAAAAGTGTTCAAC |
| | ATACACTATCTATACTCTTGATTTTGAAGGATTTAATCTACTGGTGCCGCG |
| | CGGCAGCCTCGAG |
| ModB | CCATGGGAATTATTAATCTTGCAGATGTTGAACAGTTATCTATAAAAGCTG |
| [NcoI, | AAAGCGTTGATTTTCAATATGATATGTATAAAAAGGTCTGTGAAAAATTTAC |
| XhoI] | TGACTTTGAGCAGTCTGTTCTTTGGCAATGTATGGAAGCCAAAAAGAATGA |
| | AGCTCTTCATAAGCATTTAAATGAAATCATTAAAAAGCATTTAACTAAATCG |
| | CCTTATCAATTATATCGTGGTATATCAAAATCGACAAAAGAACTCATTAAAG |
| | ATTTACAAGTTGGAGAAGTGTTTTCAACGAACAGGGTAGATTCATTTACTA |
| | CTAGTTTGCATACAGCGTGTTCTTTTTCTTATGCTGAATATTTCACTGAAAC |
| | AATACTTCGTTTAAAAACTGATAAAGCTTTTAATTATTCTGACCATATCAGC |
| | GATATTATACTTTCTTCTCCTAATACTGAGTTTAAGTACACGTATGAAGATA |
| | CTGATGGATTAGATTCAGAGCGTACTGATAACTTAATGATGATTGTGCGTG |
| | AACAAGAATGGATGATTCCAATTGGAAAGTATAAAATAACTTCTATTTCAAA |
| | AGAAAAATTACACGATTCATTTGGAACATTTAAAGTTTATGATATTGAGGTA |
| | GTTGAACTGGTGCCGCGCGGCAGCCTCGAG |
| pET28-rS1 | CCATGGGAACTGAATCTTTTGCTCAACTCTTTGAAGAGTCCTTAAAAGAAA |
| [NcoI, | TCGAAACCCGCCCGGGTTCTATCGTTCGTGGCGTTGTTGTTGCTATCGAC |
| XhoI] | AAAGACGTAGTACTGGTTGACGCTGGTCTGAAATCTGAGTCCGCCATCCC |
| | GGCTGAGCAGTTCAAAAACGCCCAGGGCGAGCTGGAAATCCAGGTAGGT |
| | GACGAAGTTGACGTTGCTCTGGACGCAGTAGAAGACGGCTTCGGTGAAA |
| | CTCTGCTGTCCCGTGAGAAAGCTAAACGTCACGAAGCCTGGATCACGCTG |
| | GAAAAAGCTTACGAAGATGCTGAAACTGTTACCGGTGTTATCAACGGCAA |
| | AGTTAAGGGCGGCTTCACTGTTGAGCTGAACGGTATTCGTGCGTTCCTGC |
| | CAGGTTCTCTGGTAGACGTTCGTCCGGTGCGTGACACTCTGCACCTGGAA |
| | GGCAAAGAGCTTGAATTTAAAGTAATCAAGCTGGATCAGAAGCGCAACAA |
| | CGTTGTTGTTTCTCGTCGTGCCGTTATCGAATCCGAAAACAGCGCAGAGC |
| | GCGATCAGCTGCTGGAAAACCTGCAGGAAGGCATGGAAGTTAAAGGTAT |
| | CGTTAAGAACCTCACTGACTACGGTGCATTCGTTGATCTGGGCGGCGTTG |
| | ACGGCCTGCTGCACATCACTGACATGGCCTGGAAACGCGTTAAGCATCC |
| | GAGCGAAATCGTCAACGTGGGCGACGAAATCACTGTTAAAGTGCTGAAGT |
| | TCGACCGCGAACGTACCCGTGTATCCCTGGGCCTGAAACAGCTGGGCGA |
| | AGATCCGTGGGTAGCTATCGCTAAACGTTATCCGGAAGGTACCAAACTGA |
| | CTGGTCGCGTGACCAACCTGACCGACTACGGCTGCTTCGTTGAAATCGAA |
| | GAAGGCGTTGAAGGCCTGGTACACGTTTCCGAAATGGACTGGACCAACA |
| | AAAACATCCACCCGTCCAAAGTTGTTAACGTTGGCGATGTAGTGGAAGTT |
| | ATGGTTCTGGATATCGACGAAGAACGTCGTCGTATCTCCCTGGGTCTGAA |
| | ACAGTGCAAAGCTAACCCGTGGCAGCAGTTCGCGGAAACCCACAACAAG |
| | GGCGACCGTGTTGAAGGTAAAATCAAGTCTATCACTGACTTCGGTATCTT |
| | CATCGGCTTGGACGGCGGCATCGACGGCCTGGTTCACCTGTCTGACATC |
| | TCCTGGAACGTTGCAGGCGAAGAAGCAGTTCGTGAATACAAAAAAGGCGA |
| | CGAAATCGCTGCAGTTGTTCTGCAGGTTGACGCAGAACGTGAACGTATCT |
| | CCCTGGGCGTTAAACAGCTCGCAGAAGATCCGTTCAACAACTGGGTTGCT |
| | CTGAACAAGAAAGGCGCTATCGTAACCGGTAAAGTAACTGCAGTTGACGC |
| | TAAAGGCGCAACCGTAGAACTGGCTGACGGCGTTGAAGGTTACCTGCGT |
| | GCTTCTGAAGCATCCCGTGACCGCGTTGAAGACGCTACCCTGGTTCTGAG |
| | CGTTGGCGACGAAGTTGAAGCTAAATTCACCGGCGTTGATCGTAAAAACC |
| | GCGCAATCAGCCTGTCTGTTCGTGCGAAAGACGAAGCTGACGAGAAAGA |
| | TGCAATCGCAACTGTTAACAAACAGGAAGATGCAAACTTCTCCAACAACG |
| | CAATGGCTGAAGCTTTCAAAGCAGCTAAAGGCGAGCTGGTGCCGCGCGG |
| | CAGCCTCGAG |
| pTAC-rS1 | ATGAAGCTTCCTCGAGAGACTGAATCTTTTGCTCAACTCTTTGAAGAGTCC |
| [XhoI, | TTAAAAGAAATCGAAACCCGCCCGGGTTCTATCGTTCGTGGCGTTGTTGT |
| SphI] | TGCTATCGACAAAGACGTAGTACTGGTTGACGCTGGTCTGAAATCTGAGT |
| | CCGCCATCCCGGCTGAGCAGTTCAAAAACGCCCAGGGCGAGCTGGAAAT |
| | CCAGGTAGGTGACGAAGTTGACGTTGCTCTGGACGCAGTAGAAGACGGC |
| | TTCGGTGAAACTCTGCTGTCCCGTGAGAAAGCTAAACGTCACGAAGCCTG |
| | GATCACGCTGGAAAAAGCTTACGAAGATGCTGAAACTGTTACCGGTGTTA |
| | TCAACGGCAAAGTTAAGGGCGGCTTCACTGTTGAGCTGAACGGTATTCGT |
| | GCGTTCCTGCCAGGTTCTCTGGTAGACGTTCGTCCGGTGCGTGACACTCT |
| | GCACCTGGAAGGCAAAGAGCTTGAATTTAAAGTAATCAAGCTGGATCAGA |
| | AGCGCAACAACGTTGTTGTTTCTCGTCGTGCCGTTATCGAATCCGAAAAC |
| | AGCGCAGAGCGCGATCAGCTGCTGGAAAACCTGCAGGAAGGCATGGAAG |
| | TTAAAGGTATCGTTAAGAACCTCACTGACTACGGTGCATTCGTTGATCTGG |
| | GCGGCGTTGACGGCCTGCTGCACATCACTGACATGGCCTGGAAACGCGT |
| | TAAGCATCCGAGCGAAATCGTCAACGTGGGCGACGAAATCACTGTTAAAG |
| | TGCTGAAGTTCGACCGCGAACGTACCCGTGTATCCCTGGGCCTGAAACA |
| | GCTGGGCGAAGATCCGTGGGTAGCTATCGCTAAACGTTATCCGGAAGGT |
| | ACCAAACTGACTGGTCGCGTGACCAACCTGACCGACTACGGCTGCTTCGT |
| | TGAAATCGAAGAAGGCGTTGAAGGCCTGGTACACGTTTCCGAAATGGACT |
| | GGACCAACAAAAACATCCACCCGTCCAAAGTTGTTAACGTTGGCGATGTA |
| | GTGGAAGTTATGGTTCTGGATATCGACGAAGAACGTCGTCGTATCTCCCT |
| | GGGTCTGAAACAGTGCAAAGCTAACCCGTGGCAGCAGTTCGCGGAAACC |
| | CACAACAAGGGCGACCGTGTTGAAGGTAAAATCAAGTCTATCACTGACTT |
| | CGGTATCTTCATCGGCTTGGACGGCGGCATCGACGGCCTGGTTCACCTG |
| | TCTGACATCTCCTGGAACGTTGCAGGCGAAGAAGCAGTTCGTGAATACAA |
| | AAAAGGCGACGAAATCGCTGCAGTTGTTCTGCAGGTTGACGCAGAACGT |
| | GAACGTATCTCCCTGGGCGTTAAACAGCTCGCAGAAGATCCGTTCAACAA |
| | CTGGGTTGCTCTGAACAAGAAAGGCGCTATCGTAACCGGTAAAGTAACTG |
| | CAGTTGACGCTAAAGGCGCAACCGTAGAACTGGCTGACGGCGTTGAAGG |
| | TTACCTGCGTGCTTCTGAAGCATCCCGTGACCGCGTTGAAGACGCTACCC |
| | TGGTTCTGAGCGTTGGCGACGAAGTTGAAGCTAAATTCACCGGCGTTGAT |
| | CGTAAAAACCGCGCAATCAGCCTGTCTGTTCGTGCGAAAGACGAAGCTGA |
| | CGAGAAAGATGCAATCGCAACTGTTAACAAACAGGAAGATGCAAACTTCT |
| | CCAACAACGCAATGGCTGAAGCTTTCAAAGCAGCTAAAGGCGAGTGCATG |
| | CACGTAGAG |
| S1 D1 | CCATGGAGTCCTTAAAAGAAATCGAAACCCGCCCGGGTTCTATCGTTCGT |
| [NcoI, | GGCGTTGTTGTTGCTATCGACAAAGACGTAGTACTGGTTGACGCTGGTCT |
| XhoI] | GAAATCTGAGTCCGCCATCCCGGCTGAGCAGTTCAAAAACGCCCAGGGC |
| | GAGCTGGAAATCCAGGTAGGTGACGAAGTTGACGTTGCTCTGGACGCAG |
| | TAGAAGACGGCTTCGGTGAAACTCTGCTGTCCCGTGAGAAAGCTAAACGT |
| | CACGAAGCCCTGGTGCCGCGCGGCAGCCTCGAG |
| S1 D2 | CCATGGCCTGGATCACGCTGGAAAAAGCTTACGAAGATGCTGAAACTGTT |
| [NcoI, | ACCGGTGTTATCAACGGCAAAGTTAAGGGCGGCTTCACTGTTGAGCTGAA |
| XhoI] | CGGTATTCGTGCGTTCCTGCCAGGTTCTCTGGTAGACGTTCGTCCGGTGC |
| | GTGACACTCTGCACCTGGAAGGCAAAGAGCTTGAATTTAAAGTAATCAAG |
| | CTGGATCAGAAGCGCAACAACGTTGTTGTTTCTCGTCGTGCCGTTATCGA |
| | ATCCGAAAACAGCGCAGAGCTGGTGCCGCGCGGCAGCCTCGAG |
| S1 D2 | CCATGGCCTGGATCACGCTGGAAAAAGCTTACGAAGATGCTGAAACTGTT |
| R139A | ACCGGTGTTATCAACGGCAAAGTTAAGGGCGGCTTCACTGTTGAGCTGAA |
| [NcoI, | CGGTATTCGTGCGTTCCTGCCAGGTTCTCTGGTAGACGTTGCCCCGGTG |
| XhoI] | CGTGACACTCTGCACCTGGAAGGCAAAGAGCTTGAATTTAAAGTAATCAA |
| | GCTGGATCAGAAGCGCAACAACGTTGTTGTTTCTCGTCGTGCCGTTATCG |
| | AATCCGAAAACAGCGCAGAGCTGGTGCCGCGCGGCAGCCTCGAG |
| S1 D2 | CCATGGCCTGGATCACGCTGGAAAAAGCTTACGAAGATGCTGAAACTGTT |
| R139K | ACCGGTGTTATCAACGGCAAAGTTAAGGGCGGCTTCACTGTTGAGCTGAA |
| [NcoI, | CGGTATTCGTGCGTTCCTGCCAGGTTCTCTGGTAGACGTTAAACCGGTGC |
| XhoI] | GTGACACTCTGCACCTGGAAGGCAAAGAGCTTGAATTTAAAGTAATCAAG |
| | CTGGATCAGAAGCGCAACAACGTTGTTGTTTCTCGTCGTGCCGTTATCGA |
| | ATCCGAAAACAGCGCAGAGCTGGTGCCGCGCGGCAGCCTCGAG |
| S1 D3 | CCATGGCCCGCGATCAGCTGCTGGAAAACCTGCAGGAAGGCATGGAAGT |
| [NcoI, | TAAAGGTATCGTTAAGAACCTCACTGACTACGGTGCATTCGTTGATCTGG |
| XhoI] | GCGGCGTTGACGGCCTGCTGCACATCACTGACATGGCCTGGAAACGCGT |
| | TAAGCATCCGAGCGAAATCGTCAACGTGGGCGACGAAATCACTGTTAAAG |
| | TGCTGAAGTTCGACCGCGAACGTACCCGTGTATCCCTGGGCCTGAAACA |
| | GCTGGGCGAAGATCCGCTGGTGCCGCGCGGCAGCCTCGAG |
| S1 D4 | CCATGGCCTGGGTAGCTATCGCTAAACGTTATCCGGAAGGTACCAAACTG |
| [NcoI, | ACTGGTCGCGTGACCAACCTGACCGACTACGGCTGCTTCGTTGAAATCGA |
| XhoI] | AGAAGGCGTTGAAGGCCTGGTACACGTTTCCGAAATGGACTGGACCAAC |
| | AAAAACATCCACCCGTCCAAAGTTGTTAACGTTGGCGATGTAGTGGAAGT |
| | TATGGTTCTGGATATCGACGAAGAACGTCGTCGTATCTCCCTGGGTCTGA |
| | AACAGTGCAAAGCTAACCCGCTGGTGCCGCGCGGCAGCCTCGAG |
| S1 D5 | CCATGGCCTGGCAGCAGTTCGCGGAAACCCACAACAAGGGCGACCGTGT |
| [NcoI, | TGAAGGTAAAATCAAGTCTATCACTGACTTCGGTATCTTCATCGGCTTGGA |
| XhoI] | CGGCGGCATCGACGGCCTGGTTCACCTGTCTGACATCTCCTGGAACGTT |
| | GCAGGCGAAGAAGCAGTTCGTGAATACAAAAAAGGCGACGAAATCGCTG |
| | CAGTTGTTCTGCAGGTTGACGCAGAACGTGAACGTATCTCCCTGGGCGTT |
| | AAACAGCTCGCAGAAGATCCGCTGGTGCCGCGCGGCAGCCTCGAG |
| S1 D6 | CCATGGCCTTCAACAACTGGGTTGCTCTGAACAAGAAAGGCGCTATCGTA |
| [NcoI, | ACCGGTAAAGTAACTGCAGTTGACGCTAAAGGCGCAACCGTAGAACTGG |
| XhoI] | CTGACGGCGTTGAAGGTTACCTGCGTGCTTCTGAAGCATCCCGTGACCG |
| | CGTTGAAGACGCTACCCTGGTTCTGAGCGTTGGCGACGAAGTTGAAGCTA |
| | AATTCACCGGCGTTGATCGTAAAAACCGCGCAATCAGCCTGTCTGTTCGT |
| | GCGAAAGACGAAGCTGACGAGAAACTGGTGCCGCGCGGCAGCCTCGAG |
| S1 domain | CCATGGCAGAAATCGAAGTGGGCCGCGTCTACACTGGTAAAGTGACCCG |
| of PNPase | TATCGTTGACTTTGGCGCATTTGTTGCCATCGGCGGCGGTAAAGAAGGTC |
| [NcoI, | TGGTCCACATCTCTCAAATCGCTGACAAACGCGTTGAGAAAGTGACCGAT |
| XhoI] | TACCTGCAGATGGGTCAGGAAGTACCGGTGAAAGTTCTGGAAGTTGATCG |
| | CCAGGGCCGTATCCGTCTGAGCATTAAAGAAGCGACTGAGCAGTCTCAAC |
| | CTGCTGCACTGGTGCCGCGCGGCAGCCTCGAG |
| pET28 | CCATGGAAAAATACGTCGCCGCGATGGTTTTGTCAGCTGCTGGCGATGCT |
| ARH1 | TTGGGATATTATAATGGAAAGTGGGAATTTCTTCAGGACGGGGAGAAAAT |
| [NcoI, | TCATCGTCAACTGGCTCAATTAGGGGGGCTGGATGCTCTGGACGTTGGC |
| XhoI] | CGTTGGCGTGTGTCTGATGATACTGTCATGCACTTGGCAACAGCCGAGGC |
| | TTTGGTCGAGGCCGGAAAGGCTCCAAAACTGACTCAGCTTTATTATTTGTT |
| | AGCCAAGCACTATCAGGATTGCATGGAAGATATGGACGGTCGCGCACCC |
| | GGGGGTGCGTCTGTACACAACGCGATGCAGCTTAAACCTGGGAAACCGA |
| | ATGGCTGGCGTATCCCATTTAACTCGCATGAAGGAGGGTGTGGCGCGGC |
| | GATGCGCGCGATGTGTATCGGTTTGCGTTTTCCGCATCACTCTCAATTAG |
| | ACACACTGATCCAAGTATCGATCGAGTCAGGACGTATGACCCATCATCAC |
| | CCGACAGGGTACCTTGGCGCACTTGCGTCCGCCTTATTCACGGCCTATG |
| | CGGTAAATAGCCGCCCTCCATTGCAGTGGGGTAAGGGACTTATGGAGCTT |
| | TTGCCAGAGGCTAAAAAATACATTGTCCAATCCGGGTACTTTGTGGAAGA |
| | AAATTTACAGCATTGGTCTTATTTTCAAACGAAGTGGGAAAACTATCTTAAA |
| | CTGCGTGGAATCTTGGACGGCGAGAGTGCTCCAACATTCCCTGAATCTTT |
| | TGGCGTTAAAGAGCGCGACCAGTTCTACACTTCGTTGTCATATAGTGGCT |
| | GGGGCGGTTCATCTGGGCATGATGCCCCCATGATCGCGTATGACGCGGT |
| | GCTGGCGGCGGGAGACTCCTGGAAAGAGCTTGCGCACCGCGCCTTCTTT |
| | CACGGAGGTGACTCGGATTCGACCGCAGCCATTGCTGGATGTTGGTGGG |
| | GCGTCATGTACGGATTTAAGGGCGTCAGCCCCAGCAACTACGAAAAATTA |
| | GAGTATCGCAATCGCCTTGAGGAAACAGCTCGCGCACTTTACTCGCTGGG |
| | TAGTAAAGAAGACACTGTTATCTCGCTGCTGGTGCCGCGCGGCAGCCTC |
| | GAG |
| pTAC | ATGAAGCTTCCTCGAGAAAAATACGTCGCCGCGATGGTTTTGTCAGCTGC |
| ARH1 | TGGCGATGCTTTGGGATATTATAATGGAAAGTGGGAATTTCTTCAGGACG |
| [XhoI, | GGGAGAAAATTCATCGTCAACTGGCTCAATTAGGGGGGCTGGATGCTCTG |
| SphI] | GACGTTGGCCGTTGGCGTGTGTCTGATGATACTGTCATGCACTTGGCAAC |
| | AGCCGAGGCTTTGGTCGAGGCCGGAAAGGCTCCAAAACTGACTCAGCTT |
| | TATTATTTGTTAGCCAAGCACTATCAGGATTGCATGGAAGATATGGACGGT |
| | CGCGCACCCGGGGGTGCGTCTGTACACAACGCGATGCAGCTTAAACCTG |
| | GGAAACCGAATGGCTGGCGTATCCCATTTAACTCGCATGAAGGAGGGTGT |
| | GGCGCGGCGATGCGCGCGATGTGTATCGGTTTGCGTTTTCCGCATCACT |
| | CTCAATTAGACACACTGATCCAAGTATCGATCGAGTCAGGACGTATGACC |
| | CATCATCACCCGACAGGGTACCTTGGCGCACTTGCGTCCGCCTTATTCAC |
| | GGCCTATGCGGTAAATAGCCGCCCTCCATTGCAGTGGGGTAAGGGACTT |
| | ATGGAGCTTTTGCCAGAGGCTAAAAAATACATTGTCCAATCCGGGTACTTT |
| | GTGGAAGAAAATTTACAGCATTGGTCTTATTTTCAAACGAAGTGGGAAAAC |
| | TATCTTAAACTGCGTGGAATCTTGGACGGCGAGAGTGCTCCAACATTCCC |
| | TGAATCTTTTGGCGTTAAAGAGCGCGACCAGTTCTACACTTCGTTGTCATA |
| | TAGTGGCTGGGGCGGTTCATCTGGGCATGATGCCCCCATGATCGCGTAT |
| | GACGCGGTGCTGGCGGCGGGAGACTCCTGGAAAGAGCTTGCGCACCGC |
| | GCCTTCTTTCACGGAGGTGACTCGGATTCGACCGCAGCCATTGCTGGATG |
| | TTGGTGGGGCGTCATGTACGGATTTAAGGGCGTCAGCCCCAGCAACTAC |
| | GAAAAATTAGAGTATCGCAATCGCCTTGAGGAAACAGCTCGCGCACTTTA |
| | CTCGCTGGGTAGTAAAGAAGACACTGTTATCTCGCTGCTGGTAGTAAAGA |
| | AGACACTGTTATCTCGCTGCTGGTGCCGCGCGGCAGCTGCATGC |
| pET ARH1 | CCATGGAAAAATACGTCGCCGCGATGGTTTTGTCAGCTGCTGGCGATGCT |
| D55,56A | TTGGGATATTATAATGGAAAGTGGGAATTTCTTCAGGACGGGGAGAAAAT |
| [NcoI, | TCATCGTCAACTGGCTCAATTAGGGGGGCTGGATGCTCTGGACGTTGGC |
| XhoI] | CGTTGGCGTGTGTCTGCGGCGACTGTCATGCACTTGGCAACAGCCGAGG |
| | CTTTGGTCGAGGCCGGAAAGGCTCCAAAACTGACTCAGCTTTATTATTTGT |
| | TAGCCAAGCACTATCAGGATTGCATGGAAGATATGGACGGTCGCGCACC |
| | CGGGGGTGCGTCTGTACACAACGCGATGCAGCTTAAACCTGGGAAACCG |
| | AATGGCTGGCGTATCCCATTTAACTCGCATGAAGGAGGGTGTGGCGCGG |
| | CGATGCGCGCGATGTGTATCGGTTTGCGTTTTCCGCATCACTCTCAATTA |
| | GACACACTGATCCAAGTATCGATCGAGTCAGGACGTATGACCCATCATCA |
| | CCCGACAGGGTACCTTGGCGCACTTGCGTCCGCCTTATTCACGGCCTAT |
| | GCGGTAAATAGCCGCCCTCCATTGCAGTGGGGTAAGGGACTTATGGAGC |
| | TTTTGCCAGAGGCTAAAAAATACATTGTCCAATCCGGGTACTTTGTGGAAG |
| | AAAATTTACAGCATTGGTCTTATTTTCAAACGAAGTGGGAAAACTATCTTAA |
| | ACTGCGTGGAATCTTGGACGGCGAGAGTGCTCCAACATTCCCTGAATCTT |
| | TTGGCGTTAAAGAGCGCGACCAGTTCTACACTTCGTTGTCATATAGTGGC |
| | TGGGGCGGTTCATCTGGGCATGATGCCCCCATGATCGCGTATGACGCGG |
| | TGCTGGCGGCGGGAGACTCCTGGAAAGAGCTTGCGCACCGCGCCTTCTT |
| | TCACGGAGGTGACTCGGATTCGACCGCAGCCATTGCTGGATGTTGGTGG |
| | GGCGTCATGTACGGATTTAAGGGCGTCAGCCCCAGCAACTACGAAAAATT |
| | AGAGTATCGCAATCGCCTTGAGGAAACAGCTCGCGCACTTTACTCGCTGG |
| | GTAGTAAAGAAGACACTGTTATCTCGCTGCTGGTGCCGCGCGGCAGCCT |
| | CGAG |
| pTAC | ATGAAGCTTCCTCGAGAAAAATACGTCGCCGCGATGGTTTTGTCAGCTGC |
| ARH1 | TGGCGATGCTTTGGGATATTATAATGGAAAGTGGGAATTTCTTCAGGACG |
| D55,56A | GGGAGAAAATTCATCGTCAACTGGCTCAATTAGGGGGGCTGGATGCTCTG |
| [XhoI, | GACGTTGGCCGTTGGCGTGTGTCTGCGGCGACTGTCATGCACTTGGCAA |
| SphI] | CAGCCGAGGCTTTGGTCGAGGCCGGAAAGGCTCCAAAACTGACTCAGCT |
| | TTATTATTTGTTAGCCAAGCACTATCAGGATTGCATGGAAGATATGGACGG |
| | TCGCGCACCCGGGGGTGCGTCTGTACACAACGCGATGCAGCTTAAACCT |
| | GGGAAACCGAATGGCTGGCGTATCCCATTTAACTCGCATGAAGGAGGGT |
| | GTGGCGCGGCGATGCGCGCGATGTGTATCGGTTTGCGTTTTCCGCATCA |
| | CTCTCAATTAGACACACTGATCCAAGTATCGATCGAGTCAGGACGTATGA |
| | CCCATCATCACCCGACAGGGTACCTTGGCGCACTTGCGTCCGCCTTATTC |
| | ACGGCCTATGCGGTAAATAGCCGCCCTCCATTGCAGTGGGGTAAGGGAC |
| | TTATGGAGCTTTTGCCAGAGGCTAAAAAATACATTGTCCAATCCGGGTACT |
| | TTGTGGAAGAAAATTTACAGCATTGGTCTTATTTTCAAACGAAGTGGGAAA |
| | ACTATCTTAAACTGCGTGGAATCTTGGACGGCGAGAGTGCTCCAACATTC |
| | CCTGAATCTTTTGGCGTTAAAGAGCGCGACCAGTTCTACACTTCGTTGTCA |
| | TATAGTGGCTGGGGCGGTTCATCTGGGCATGATGCCCCCATGATCGCGT |
| | ATGACGCGGTGCTGGCGGCGGGAGACTCCTGGAAAGAGCTTGCGCACC |
| | GCGCCTTCTTTCACGGAGGTGACTCGGATTCGACCGCAGCCATTGCTGG |
| | ATGTTGGTGGGGCGTCATGTACGGATTTAAGGGCGTCAGCCCCAGCAAC |
| | TACGAAAAATTAGAGTATCGCAATCGCCTTGAGGAAACAGCTCGCGCACT |
| | TTACTCGCTGGGTAGTAAAGAAGACACTGTTATCTCGCTGCTGGTGCCGC |
| | GCGGCAGCTGCATGC |

Extended Data tables 3: Primers used in this study. Corresponding restriction site in bold, underlined; mutation in bold and italic

| Primer | Sequence (5′ to 3′) |
|---|---|
| Fwd Alt NcoI | ATCGACCCATGGGAGAACTTATTACAGAATTATTTGACG |
| Rev Alt XhoI | ATTCGACTCGAGGCTGCCGCGCGGCACCAGTCCTTGAACGAA |
| | CTTGTAAGGCATG |
| Fwd ModA NcoI | ATCGACCATGGGAAAATACTCAGTAATGCAACTAAAAG |
| Rev ModA XhoI | ATCGTACTCGAGGCTGCCGCGCGGCACCAGTAGATTAAATCC |
| | TTCAAAATCAAG |
| Fwd ModB NcoI | ATCGACCCATGGGAATTATTAATCTTGCAGATGTTG |
| Rev ModB XhoI | ACTTAGCTCGAGGCTGCCGCGCGGCACCAGTTCAACTACCTC |
| | AATATCATAAAC |
| Fwd rS1 NcoI | ATCGACCCATGGGAACTGAATCTTTTGCTCAACTCTTTGAAGA |
| | GTCC |
| Rev rS1 XhoI | ATTCGACTCGAGGCTGCCGCGCGGCACCAGCTCGCCTTTAGC |
| | TGCTTTG |
| Fwd rS1-pTAC XhoI | ATG AAG CTT CCT CGA |
| | GAGACTGAATCTTTTGCTCAACTCTTTGAAGAGTCC |
| Rev rS1-pTAC Sphl | CTCTACGTGCATGCACTCGCCTTTAGCTGCTTTGAAAGCTTCA |
| | GCC |
| Fwd NcoI rS1 D1 | ATCGACCCATGGAGTCCTTAAAAGAAATCGAAACCCGCCCGG |
| | G |
| Rev XhoI rS1 D1 | TGGTGCTCGAGGCTGCCGCGCGGCACCAGGGCTTCGTGACG |
| | TTTAGCTTTCTCACGGG |
| Fwd NcoI rS1 D2 | ATCGACCCATGGCCTGGATCACGCTGGAAAAAGCTTACGAAG |
| | ATGCTGAAAC |
| Rev XhoI rS1 D2 | GGTGCTCGAGGCTGCCGCGCGGCACCAGCTCTGCGCTGTTTT |
| | CGGATTCGATAACGGCAC |
| Fwd NcoI rS1 D3 | ATCGACCCATGGCCCGCGATCAGCTGCTGGAAAACCTGCAGG |
| | AAGG |
| Rev XhoI rS1 D3 | TGGTGCTCGAGGCTGCCGCGCGGCACCAGCGGATCTTCGCC |
| | CAGCTGTTTCAGGCCCAGG |
| Fwd NcoI rS1 D4 | ATCGACCCATGGCCTGGGTAGCTATCGCTAAACGTTATCCGGA |
| | AGG |
| Rev XhoI rS1 D4 | TGGTGCTCGAGGCTGCCGCGCGGCACCAGCGGGTTAGCTTT |
| | GCACTGTTTCAGACCCAGGGAG |
| Fwd NcoI rS1 D5 | ATCGACCCATGGCCTGGCAGCAGTTCGCGGAAACCCACAACA |
| | AGGGCGACCGTGTTG |
| Rev XhoI S1 D5 | TGGTGCTCGAGGCTGCCGCGCGGCACCAGCGGATOTTCTGC |
| | GAGCTGTTTAACGCCCAGGGAGATACG |
| Fwd NcoI rS1 D6 | ATCGACCCATGGCCTTCAACAACTGGGTTGCTCTGAACAAGAA |
| | AGGCGCTATCG |
| Rev XhoI rS1 D6 | TGGTGCTCGAGGCTGCCGCGCGGCACCAGTTTCTCGTCAGCT |
| | TCGTCTTTCGCACGAACAGACAGG |
| Fwd NcoI PNPase | ATCGACCCATGGCAGAAATCGAAGTGGGCCGCGTCTACACTG |
| rS1 binding | GTAAAGTGACCCG |
| Rev XhoI PNPase | TGGTGCTCGAGGCTGCCGCGCGGCACCAGTGCAGCAGGTTG |
| rS1 binding | AGACTGCTCAGTCGCTTC |
| Fwd ARH1 NcoI | TGCAGCCATGGAAAAATACGTCGCCGCGATG |
| Rev ARH1 XhoI | GTGGTGCTCGAGGCTGCCGCGCGGCACCAG |
| Fwd XhoI pTAC | ATG AAG CTT CCT CGA GAA AAA TAC GTC GCC GCG ATG |
| ARH1 | GTT TTG TCA GCT GCT GGC |
| Rev Sphl pTAC | CTACGTGCATGCAGCTGCCGCGCGGCACCAGCAGCGAGATAA |
| ARH1 | CAGTGTCTTCTTTACTACC |
| Fwd rS1 R139A | CTGGTAGACGTTGCCCCGGTGCGTGACACTC |
| Fwd rS1 R139K | CTGGTAGACGTTAAACCGGTGCGTGACACTC |
| Rev rS1 R139 | AGAACCTGGCAGGAACGCACGAATACCG |
| Fwd ARH1 D55,56A | 5′-P- |
| | GGCCGTTGGCGTGTGTCTGCGGCGACTGTCATGCACTTGGC |
| Rev ARH1 D55,56A | 5′-P-AACGTCCAGAGCATCCAGCCCCCCTAA |

Extended Data table 4: Strains and plasmids used in this study

| Name | Description | Reference or resource |
|---|---|---|
| E. coli strain B | E. coli strain applied for | DMSZ |
| | bacteriophage T4 infection | |
| E. coli strain B pTAC rS1 | E. coli strain B expressing His- | This study |
| | tagged rS1 under control of | |
| | E. coli RNA polymerase | |
| | promoter | |
| E. coli strain B pATC | E. coli strain B expressing His- | This study |
| ARH1 | tagged ARH1 under control of E. | |
| | coli RNA polymerase promoter | |
| E. coli strain B pTAC | E. coli strain B expressing His- | This study |
| ARH1 D55,56A | tagged ARH1 inactive mutant | |
| | under control of E. coli RNA | |
| | polymerase promoter | |
| E. coli strain FLAG-S1 | E. coli strain with endogenous | Strain was a kind gift from |
| (Ced 64) | expression of rS1 with a | Prof. Dr. Gerhart Wagner ³ |
| | 3xFLAG at C-terminus | |
| E. coli BL21 (DE3) pET16 | E. coli strain expressing His- | Plasmid was a kind gift |
| RNase E (1-529) | tagged catalytic domain of | from Prof. Dr. Ben Luisi ⁴ |
| | RNase E (1-529) | |
| E. coli BL21 (DE3) pET 28 | E. coli strain expressing His- | ⁵ |
| NudC V157A, E174A, | tagged inactive Mutant of NudC | |
| E177A, E178A | | |
| E. coli BL21 (DE3) pET 28 | E. coli strain expressing His- | This study |
| rS1 | tagged rS1 | |
| E. coli BL21 (DE3) pET 28 | E. coli strain expressing His- | This study |
| rS1 R139K | tagged rS1 R139K variant | |
| E. coli BL21 (DE3) pET 28 | E. coli strain expressing His- | This study |
| rS1 R139A | tagged rS1 R139A variant | |
| E. coli BL21 (DE3) pET 28 | E. coli strain expressing His- | This study |
| rS1 D1 | tagged rS1 D1 | |
| E. coli BL21 (DE3) pET 28 | E. coli strain expressing His- | This study |
| rS1 D2 | tagged rS1 D2 | |
| E. coli BL21 (DE3) pET 28 | E. coli strain expressing His- | This study |
| rS1 D2 R139K | tagged rS1 D2 R139K | |
| E. coli BL21 (DE3) pET 28 | E. coli strain expressing His- | This study |
| rS1 D2 R139A | tagged rS1 D2 R139A | |
| E. coli BL21 (DE3) pET 28 | E. coli strain expressing His- | This study |
| rS1 D3 | tagged rS1 D3 | |
| E. coli BL21 (DE3) pET 28 | E. coli strain expressing His- | This study |
| rS1 D4 | tagged rS1 D4 | |
| E. coli BL21 (DE3) pET 28 | E. coli strain expressing His- | This study |
| rS1 D5 | tagged r$1 D5 | |
| E. coli BL21 (DE3) pET 28 | E. coli strain expressing His- | This study |
| rS1 D6 | tagged rS1 D6 | |
| E. coli BL21 (DE3) pET 28 | E. coli strain expressing His- | This study |
| Alt | tagged Alt | |
| E. coli BL21 (DE3) pET 28 | E. coli strain expressing His- | This study |
| ModA | tagged ModA | |
| E. coli BL21 (DE3) pET 28 | E. coli strain expressing His- | This study |
| ModB | tagged ModB | |
| E. coli BL21 (DE3) pET 28 | E. coli strain expressing His- | ⁵ |
| NudC | tagged NudC | |
| E. coli BL21 (DE3) pET 28 | E. coli strain expressing His- | This study |
| PNPase S1 domain | tagged PNPase S1 domain | |
| E. coli BL21 (DE3) pET 28 | E. coli strain expressing His- | This study |
| ARH1 | tagged ARH1 | |
| E. coli BL21 (DE3) pET 28 | E. coli strain expressing His- | This study |
| ARH1 D55A D56A | tagged ARH1 D55A D56A | |

### REFERENCES

ADDIN EN.REFLIST 1 Cohen, M. S. & Chang, P. Insights into the biogenesis, function, and regulation of ADP-ribosylation. Nat Chem Biol 14, 236-243, doi:10.1038/nchembio.2568 (2018).
2 Tiemann, B. et al. ModA and ModB, two ADP-ribosyltransferases encoded by bacteriophage T4: catalytic properties and mutation analysis. J Bacteriol 186, 7262-7272, doi:10.1128/JB.186.21.7262-7272.2004 (2004).
3 Koch, T., Raudonikiene, A., Wilkens, K. & Rüger, W. Overexpression, purification, and characterisation of the ADP-ribosyltransferase (gpAlt) of bacteriophage T4: ADP-ribosylation of E. coli RNA polymerase modulates T4 ‘early’ transcription. Gene Expr 4, 253-264 (1995).
4 Cahova, H., Winz, M. L., Höfer, K., Nübel, G. & Jäschke, A. NAD captureSeq indicates NAD as a bacterial cap for a subset of regulatory RNAs. Nature 519, 374-377, doi:10.1038/nature14020 (2015).
5 Jiao, X. et al. 5′ End Nicotinamide Adenine Dinucleotide Cap in Human Cells Promotes RNA Decay through DXO-Mediated deNADding. Cell 168, 1015-1027 e1010, doi:10.1016/j.cell.2017.02.019 (2017).
6 Chen, Y. G., Kowtoniuk, W. E., Agarwal, I., Shen, Y. & Liu, D. R. LC/MS analysis of cellular RNA reveals NAD-linked RNA. Nat Chem Biol 5, 879-881, doi:10.1038/nchembio.235 (2009).
7 Fehr, A. R. et al. The impact of PARPs and ADP-ribosylation on inflammation and host-pathogen interactions. Genes Dev 34, 341-359, doi:10.1101/gad.334425.119 (2020).
8 Simon, N. C., Aktories, K. & Barbieri, J. T. Novel bacterial ADP-ribosylating toxins: structure and function. Nat Rev Microbiol 12, 599-611, doi:10.1038/nrmicro3310 (2014).
9 Luscher, B. et al. ADP-Ribosylation, a Multifaceted Post-translational Modification Involved in the Control of Cell Physiology in Health and Disease. Chem Rev 118, 1092-1136, doi:10.1021/acs.chemrev.7b00122 (2018).
10 Morales-Filloy, H. G. et al. The 5′ NAD Cap of RNAIII Modulates Toxin Production in Staphylococcus aureus Isolates. J Bacteriol 202, doi:10.1128/JB.00591-19 (2020).
11 Frindert, J. et al. Identification, Biosynthesis, and Decapping of NAD-Capped RNAs in B. subtilis. Cell Rep 24, 1890-1901 e1898, doi:10.1016/j.celrep.2018.07.047 (2018).
12 Walters, R. W. et al. Identification of NAD+ capped mRNAs in Saccharomyces cerevisiae. Proc Natl Acad Sci USA 114, 480-485, doi:10.1073/pnas.1619369114 (2017).
13 Wang, Y. et al. NAD(+)-capped RNAs are widespread in the Arabidopsis transcriptome and can probably be translated. Proc Natl Acad Sci USA 116, 12094-12102, doi:10.1073/pnas.1903682116 (2019).
14 Zhang, Y. et al. Extensive 5′-surveillance guards against non-canonical NAD-caps of nuclear mRNAs in yeast. Nat Commun 11, 5508, doi:10.1038/s41467-020-19326-3 (2020).
15 Hofer, K. & Jäschke, A. Epitranscriptomics: RNA Modifications in Bacteria and Archaea. Microbiol Spectr 6, doi:10.1128/microbiolspec.RWR-0015-2017 (2018).
16 Miller, E. S. et al. Bacteriophage T4 genome. Microbiol Mol Biol Rev 67, 86-156, table of contents, doi:10.1128/MMBR.67.1.86-156.2003 (2003).
17 Rohrer, H., Zillig, W. & Mailhammer, R. ADP-ribosylation of DNA-dependent RNA polymerase of Escherichia coli by an NAD+: protein ADP-ribosyltransferase from bacteriophage T4. Eur J Biochem 60, 227-238, doi:10.1111/j.1432-1033.1975.tb20995.x (1975).
18 Depping, R., Lohaus, C., Meyer, H. E. & Ruger, W. The mono-ADP-ribosyltransferases Alt and ModB of bacteriophage T4: target proteins identified. Biochem Bioph Res Co 335, 1217-1223, doi:10.1016/j.bbrc.2005.08.023 (2005).
19 Skorko, R., Zillig, W., Rohrer, H., Fujiki, H. & Mailhammer, R. Purification and properties of the NAD+: protein ADP-ribosyltransferase responsible for the T4-phage-induced modification of the alpha subunit of DNA-dependent RNA polymerase of Escherichia coli. Eur J Biochem 79, 55-66, doi:10.1111/j.1432-1033.1977.tb11783.x (1977).
20 Tiemann, B., Depping, R. & Ruger, W. Overexpression, purification, and partial characterisation of ADP-ribosyltransferases modA and modB of bacteriophage T4. Gene Expr 8, 187-196 (1999).
21 Hofer, K. et al. Structure and function of the bacterial decapping enzyme NudC. Nat Chem Biol 12, 730-734, doi:10.1038/nchembio.2132 (2016).
22 Goelz, S. & Steitz, J. A. Escherichia coli ribosomal protein S1 recognises two sites in bacteriophage Qbeta RNA. J Biol Chem 252, 5177-5179 (1977).
23 Cervantes-Laurean, D., Jacobson, E. L. & Jacobson, M. K. Glycation and glycoxidation of histones by ADP-ribose. J Biol Chem 271, 10461-10469, doi:10.1074/jbc.271.18.10461 (1996).
24 Oka, S., Kato, J. & Moss, J. Identification and characterisation of a mammalian 39-kDa poly(ADP-ribose) glycohydrolase. J Biol Chem 281, 705-713, doi:10.1074/jbc.M510290200 (2006).
25 Bycroft, M., Hubbard, T. J., Proctor, M., Freund, S. M. & Murzin, A. G. The solution structure of the S1 RNA binding domain: a member of an ancient nucleic acid-binding fold. Cell 88, 235-242, doi:10.1016/s0092-8674(00)81844-9 (1997).
26 Hentze, M. W., Castello, A., Schwarzl, T. & Preiss, T. A brave new world of RNA-binding proteins. Nat Rev Mol Cell Biol 19, 327-341, doi:10.1038/nrm.2017.130 (2018).
27 Ke, Y., Zhang, J., Lv, X., Zeng, X. & Ba, X. Novel insights into PARPs in gene expression: regulation of RNA metabolism. Cell Mol Life Sci 76, 3283-3299, doi:10.1007/s00018-019-03120-6 (2019).
28 Otten, E. G. et al. Ubiquitylation of lipopolysaccharide by RNF213 during bacterial infection. Nature, doi:10.1038/s41586-021-03566-4 (2021).
29 Flynn, R. A. et al. Small RNAs are modified with N-glycans and displayed on the surface of living cells. Cell, doi:10.1016/j.cell.2021.04.023 (2021).
30 Tsuge, H. et al. Structural basis of actin recognition and arginine ADP-ribosylation by Clostridium perfringens iota-toxin. Proc Natl Acad Sci USA 105, 7399-7404, doi:10.1073/pnas.0801215105 (2008).

1. A method for attaching a 5′-nicotinamidnucleobasedinucleotide (NND)-capped nucleic acid sequence to a fusion protein or to a complex, comprising
(a) contacting (i) a heterologous fusion protein which comprises a polypeptide of interest being fused to a tag, or (ii) a complex wherein a protein is under physiological conditions complexed with a tag with the 5′-NND-capped nucleic acid sequence and an ADP-ribosyltransferase (ART) under conditions wherein the 5′-NND-capped nucleic acid sequence is covalently attached to the tag,
wherein the tag comprises a recognition motif of the ART and preferably comprises or consists of
(i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved;
(ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved;
(iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved;
(iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved;
(v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or
(vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.

2. The method of claim 1, wherein the nucleobase of the NND is a purine base or a pyrimidine base and is preferably selected from adenine, guanine, cytosine, thymine, and uracil.

3. The method of claim 1, wherein the ART comprises or consists of SEQ ID NO: 9 or SEQ ID NO: 10 or a sequence being at least 80% identical thereto.

4. The method of claim 1, wherein the method comprises prior to step (a)
(a′) fusing the tag as defined in claim 1 to a polypeptide of interest, whereby a heterologous fusion protein which comprises a polypeptide of interest being fused to the tag is obtained, or
(a′) complexing the tag as defined in claim 1 with a poly(peptide) of interest.

5. A fusion protein comprising a polypeptide of interest being fused to a or a complex comprising a polypeptide of interest being complexed with a tag produced by the method of claim 1.

6. The fusion protein or complex of claim 5, wherein a nucleic acid sequence is covalently attached through nicotinamide nucleobase dinucleotide (NND) at its 5′-end to the tag, preferably to the side chain of the conserved Arg of the tag.

7. A composition comprising a fusion protein or complex produced by the method of claim 1.

8-12. (canceled)

13. A Kit for attaching a 5′-nicotinamidnucleobasedinucleotide (NND)-capped nucleic acid sequence to a polypeptide of interest, wherein the kit comprises
(a) the tag as defined in claim 1,
(b) an ADP-ribosyltransferase (ART) being capable of covalently attaching a 5′-NND-capped nucleic acid sequence to the tag or a nucleic acid molecule encoding said ART, and
(c) optionally instructions how to covalently attach the tag with the ART to the (poly)peptide of interest.

14. The kit of claim 13, further comprising a reaction buffer or buffer stock solution, preferably wherein the reaction buffer or the final reaction buffer to be prepared from the buffer stock solution comprises
Mg(OAc)₂ at a concentration of 50-200 mM;
NH₄Cl at a concentration of 100-500 mM;
Tris-acetate pH 7.5 at a concentration of 250-1000 mM
EDTA at a concentration of 5-15 mM;
β-mercaptoethanol at a concentration of 50-200 mM; and
glycerol at a concentration of 5-15%.

15. The kit of claim 13, further comprising one or more of MgCl₂ at least 0.25 M, preferably at a concentration of 0.5 M to 2 M,
imidazolide nicotinamide mononucleotide (Im-NMN),
nuclease free water, and
a positive control, preferably an oligonucleotide that comprises at its 3′-end a fluorescent label and/or a control fusion protein comprising a control polypeptide being fused to or complexed with a tag,
wherein the tag comprises a recognition motif of the ART and preferably comprises or consists of
(i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved;
(ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved;
(iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved;
(iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved;
(v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or
(vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.

16. The kit of claim 14, further comprising one or more of
MgCl₂ at least 0.25 M, preferably at a concentration of 0.5 M to 2 M,
imidazolide nicotinamide mononucleotide (Im-NMN),
nuclease free water, and
a positive control, preferably an oligonucleotide that comprises at its 3′-end a fluorescent label and/or a control fusion protein comprising a control polypeptide being fused to or complexed with a tag,
wherein the tag comprises a recognition motif of the ART and preferably comprises or consists of
(i) SEQ ID NO: 1 or a sequence being at least 80% identical thereto provided that the underlined Arg in the amino acid motif DVRPVRD (SEQ ID NO: 7) is conserved and preferably SEQ ID NO: 7 is conserved;
(ii) SEQ ID NO: 2 or a sequence being at least 80% identical thereto provided that the underlined Arg in the amino acid motif DVRPVRD (SEQ ID NO: 7) is conserved and preferably SEQ ID NO: 7 is conserved;
(iii) SEQ ID NO: 3 or a sequence being at least 80% identical thereto provided that the underlined Arg in the amino acid motif DVRPVRD (SEQ ID NO: 7) is conserved and preferably SEQ ID NO: 7 is conserved;
(iv) SEQ ID NO: 4 or a sequence being at least 80% identical thereto provided that the underlined Arg in the amino acid motif LADGVEGYLRASEASRDRVE (SEQ ID NO: 8) is conserved and preferably SEQ ID NO: 8 is conserved;
(v) SEQ ID NO: 5 or a sequence being at least 80% identical thereto provided that the underlined Arg in the amino acid motif LADGVEGYLRASEASRDRVE (SEQ ID NO: 8) is conserved and preferably SEQ ID NO: 8 is conserved; or
(vi) SEQ ID NO: 6 or a sequence being at least 80% identical thereto provided that the underlined Arg in the amino acid motif LADGVEGYLRASEASRDRVE (SEQ ID NO: 8) is conserved and preferably SEQ ID NO: 8 is conserved.


SEQUENCE LISTING (76 sequences)

SEQ ID NO: 1 - protein, 97 aa, Artificial Sequence
  S1 DII
MAWITLEKAYEDAETVTGVINGKVKGGFTVELNGIRAFLPGSLVDVRPVRDTLHLEGKELEFKVIKLDQKRNNVVVSRRAVIESENSAELVPRGSLE

SEQ ID NO: 2 - protein, 64 aa, Artificial Sequence
  S1 DII MM
MAETVTGVINGKVKGGFTVELNGIRAFLPGSLVDVRPVRDTLHLEGKELEFKVIKLDQKRNNLE

SEQ ID NO: 3 - protein, 80 aa, Artificial Sequence
  S1 DII Vok
MAETVTGVINGKVKGGFTVELNGIRAFLPGSLVDVRPVRDTLHLEGKELEFKVIKLDQKRNNVVVSRRAVIESENSAELE

SEQ ID NO: 4 - protein, 98 aa, Artificial Sequence
  S1 DVI
MAFNNWVALNKKGAIVTGKVTAVDAKGATVELADGVEGYLRASEASRDRVEDATLVLSVGDEVEAKFTGVDRKNRAISLSVRAKDEADEKLVPRGSLE

SEQ ID NO: 5 - protein, 67 aa, Artificial Sequence
  S1 DVI MM
MGAIVTGKVTAVDAKGATVELADGVEGYLRASEASRDRVEDATLVLSVGDEVEAKFTGVDRKNRALE

SEQ ID NO: 6 - protein, 81 aa, Artificial Sequence
  S1 DVI Vok
MGAIVTGKVTAVDAKGATVELADGVEGYLRASEASRDRVEDATLVLSVGDEVEAKFTGVDRKNRAISLSVRAKDEADEKLE

SEQ ID NO: 7 - protein, 7 aa, Artificial Sequence
  Loop S1 DII
DVRPVRD

SEQ ID NO: 8 - protein, 20 aa, Artificial Sequence
  Loop S1 DVI
LADGVEGYLRASEASRDRVE

SEQ ID NO: 9 - protein, 207 aa, Artificial Sequence
  ModB
MIINLADVEQLSIKAESVDFQYDMYKKVCEKFTDFEQSVLWQCMEAKKNEALHKHLNEIIKKHLTKSPYQLYRGISKSTKELIKDLQVGEVFSTNRVDSFTTSLHTACSFSYAEYFTETILRLKTDKAFNYSDHISDIILSSPNTEFKYTYEDTDGLDSERTDNLMMIVREQEWMIPIGKYKITSISKEKLHDSFGTFKVYDIEVVE

SEQ ID NO: 10 - protein, 222 aa, Artificial Sequence
  Mod B with His-Tag
MGIINLADVEQLSIKAESVDFQYDMYKKVCEKFTDFEQSVLWQCMEAKKNEALHKHLNEIIKKHLTKSPYQLYRGISKSTKELIKDLQVGEVFSTNRVDSFTTSLHTACSFSYAEYFTETILRLKTDKAFNYSDHISDIILSSPNTEFKYTYEDTDGLDSERTDNLMMIVREQEWMIPIGKYKITSISKEKLHDSFGTFKVYDIEVVELVPRGSLEHHHHHH

SEQ ID NO: 11 - protein, 14 aa, Artificial Sequence
  rS1 peptide
AFLPGSLVDVRPVR

SEQ ID NO: 12 - RNA, 10 nt, Artificial Sequence
  10-mer
agacuucgac

SEQ ID NO: 13 - RNA, 100 nt, Artificial Sequence
  Q.beta. (100-mer)
aucuugauacuaccuuuaguucguuuaaacacguucuugauaguaucuuuuuauuaacccaacgcguaaagcguugaaacuuugggucaauuugaucaug

SEQ ID NO: 14 - DNA, 2075 bp, Artificial Sequence
  Alt [NcoI, XhoI]
ccatgggagaacttattacagaattatttgacgaagatactactcttccaattacaaacttatatccaaagaagaaaataccgcaaattttttcagttcatgttgatgatgcaattgaacaaccaggctttcgtttatgtacctatacatctggaggtgatactaatcgtgatttaaagatgggcgataaaatgatgcatattgttccttttacattaactgctaaaggttcaattgctaaattaaaaggtcttggtccaagcccaattaattatatcaattcagtttttactgttgcaatgcaaacaatgcgccagtataaaattgatgcctgtatgctccgtattcttaagtctaaaactgctggccaagctcgacaaattcaagttattgctgatagacttatccgtagtcgttcaggtggtagatacgtccttcttaaggaactctgggattacgataaaaagtatgcatatattcttatacatcgcaaaaatgtatcactagaagacattccaggagttccggaaattagtaccgagctctttactaaagttgaatcgaaggtcggtgatgtttatatcaataaagatactggggctcaagtaactaaaaatgaggcaattgcagcatctattgcgcaagaaaatgataaacgttctgaccaagctgtaatcgttaaagttaaaatttcccgtagagcaattgcgcaaagtcagtcattggaatcttctagatttgaaacaccaatgtttcaaaaatttgaggcttcagcggccgaattaaataaaccagcggacgcgcctttaatttctgattctaatgaattaacggtaatttctacttcaggatttgcactagagaatgctcttagcagtgttacagctgggatggcattcagagaagcttctataattcctgaagataaagaatccattattaacgcagaaataaaaaataaagctttagaaagattacgaaaagaatctattacttcaataaaaaccttagaaactattgcttctatcgtcgatgatactttagaaaaatataagggtgcttggtttgaaagaaatattaacaaacattcgcatttaaaccaagatgctgcaaatgagttagtacaaaattcttggaatgcaataaaaacaaagattattcgaagagaattacgtggatatgctcttaccgctggatggtcattacatcctatagtcgaaaataaagattcatctaaatacacaccagcgcaaaaacgcggaattcgtgaatacgtaggttcaggatatgtagacataaataatgctcttttgggattatataatccagatgagcgtacaagtattttgacagcatctgacatagaaaaagctattgataatttagattcagcctttaaaaatggtgaacgattaccaaaaggtattactttgtatcgttcacaacgaatgttaccttcaatatacgaagcaatggtaaaaaatcgagttttttattttagaaactttgtgtcaacatcattatatccaaatatttttggtacttggatgactgattcatctataggtgttttaccagacgaaaagcgtttaagcgtttctattgataaaactgatgaaggacttgtaaattctagcgataatttagttggaattggatgggttattactggggctgataaggtcaatgttgttttacccggtggaagtttagcgccttcaaatgaaatggaagtcattttgccacgtggattaatggtcaaagttaataaaataaccgatgcatcttacaatgatggaacagttaaaactaacaacaagcttattcaagctgaagttatgaccacagaagaactcaccgaatcggtaatctatgacggagaccatttaatggaaactggtgaattggttacaatgacaggtgatatagaagatagagttgactttgcatcatttgtttcatcaaatgttaaacagaaagtagaatcatctcttggaattattgcgtcttgcatagatattgcaaacatgccttacaagttcgttcaaggactggtgccgcgcggcagcctcgag

SEQ ID NO: 15 - DNA, 629 bp, Artificial Sequence
  ModA [NcoI, XhoI]
ccatgggaaaatactcagtaatgcaactaaaagattttaaaataaaatcaatggatgcatcggtgcgtgcttctattcgtgaagaattactttctgaagggtttaatttatctgaaattgaacttttaattcattgtattactaataaaccagatgaccattcttggttaaatgaaataatcaaatctcgtttggttccaaacgataaacctctttggagaggtgttccagctgagactaaacaagtattaaatcaaggaattgatattattacatttgataaagtcgtatcagcttcatatgataaaaatatagctctacattttgcttctggtttagagtataacacacaagttatttttgaattcaaagctcctatggtattcaatttccaggagtatgctataaaagctctacgctgtaaagaatacaatccaaactttaagtttccggatagtcatcgttatcgtaatatggaattagtttcagatgaacaagaagtaatgataccagctggaagtgtatttagaattgcagatagatatgagtataaaaagtgttcaacatacactatctatactcttgattttgaaggatttaatctactggtgccgcgcggcagcctcgag

SEQ ID NO: 16 - DNA, 650 bp, Artificial Sequence
  ModB [NcoI, XhoI]
ccatgggaattattaatcttgcagatgttgaacagttatctataaaagctgaaagcgttgattttcaatatgatatgtataaaaaggtctgtgaaaaatttactgactttgagcagtctgttctttggcaatgtatggaagccaaaaagaatgaagctcttcataagcatttaaatgaaatcattaaaaagcatttaactaaatcgccttatcaattatatcgtggtatatcaaaatcgacaaaagaactcattaaagatttacaagttggagaagtgttttcaacgaacagggtagattcatttactactagtttgcatacagcgtgttctttttcttatgctgaatatttcactgaaacaatacttcgtttaaaaactgataaagcttttaattattctgaccatatcagcgatattatactttcttctcctaatactgagtttaagtacacgtatgaagatactgatggattagattcagagcgtactgataacttaatgatgattgtgcgtgaacaagaatggatgattccaattggaaagtataaaataacttctatttcaaaagaaaaattacacgattcatttggaacatttaaagtttatgatattgaggtagttgaactggtgccgcgcggcagcctcgag

SEQ ID NO: 17 - DNA, 1700 bp, Artificial Sequence
  pET28-rS1 [NcoI, XhoI]
ccatgggaactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggactggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagctggtgccgcgcggcagcctcgag

SEQ ID NO: 18 - DNA, 1701 bp, Artificial Sequence
  pTAC-rS1 [XhoI, SphI]
atgaagcttcctcgagagactgaatcttttgctcaactctttgaagagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagcgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgtgggtagctatcgctaaacgttatccggaaggtaccaaactgactggtcgcgtgaccaacctgaccgactacggctgcttcgttgaaatcgaagaaggcgttgaaggcctggtacacgtttccgaaatggactggaccaacaaaaacatccacccgtccaaagttgttaacgttggcgatgtagtggaagttatggttctggatatcgacgaagaacgtcgtcgtatctccctgggtctgaaacagtgcaaagctaacccgtggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtgcatgcacgtagag

SEQ ID NO: 19 - DNA, 281 bp, Artificial Sequence
  S1 D1 [NcoI, XhoI]
ccatggagtccttaaaagaaatcgaaacccgcccgggttctatcgttcgtggcgttgttgttgctatcgacaaagacgtagtactggttgacgctggtctgaaatctgagtccgccatcccggctgagcagttcaaaaacgcccagggcgagctggaaatccaggtaggtgacgaagttgacgttgctctggacgcagtagaagacggcttcggtgaaactctgctgtcccgtgagaaagctaaacgtcacgaagccctggtgccgcgcggcagcctcgag

SEQ ID NO: 20 - DNA, 293 bp, Artificial Sequence
  S1 D2 [NcoI, XhoI]
ccatggcctggatcacgctggaaaaagcttacgaagatgctgaaactgttaccggtgttatcaacggcaaagttaagggcggcttcactgttgagctgaacggtattcgtgcgttcctgccaggttctctggtagacgttcgtccggtgcgtgacactctgcacctggaaggcaaagagcttgaatttaaagtaatcaagctggatcagaagcgcaacaacgttgttgtttctcgtcgtgccgttatcgaatccgaaaacagcgcagagctggtgccgcgcggcagcctcgag

SEQ ID NO: 21 - DNA, 293 bp, Artificial Sequence
  S1 D2 R139A [NcoI, XhoI]
ccatggcctggatcacgctggaaaaagcttacgaagatgctgaaactgttaccggtgttatcaacggcaaagttaagggcggcttcactgttgagctgaacggtattcgtgcgttcctgccaggttctctggtagacgttgccccggtgcgtgacactctgcacctggaaggcaaagagcttgaatttaaagtaatcaagctggatcagaagcgcaacaacgttgttgtttctcgtcgtgccgttatcgaatccgaaaacagcgcagagctggtgccgcgcggcagcctcgag

SEQ ID NO: 22 - DNA, 293 bp, Artificial Sequence
  S1 D2 R139K [NcoI, XhoI]
ccatggcctggatcacgctggaaaaagcttacgaagatgctgaaactgttaccggtgttatcaacggcaaagttaagggcggcttcactgttgagctgaacggtattcgtgcgttcctgccaggttctctggtagacgttaaaccggtgcgtgacactctgcacctggaaggcaaagagcttgaatttaaagtaatcaagctggatcagaagcgcaacaacgttgttgtttctcgtcgtgccgttatcgaatccgaaaacagcgcagagctggtgccgcgcggcagcctcgag

SEQ ID NO: 23 - DNA, 287 bp, Artificial Sequence
  S1 D3 [NcoI, XhoI]
ccatggcccgcgatcagctgctggaaaacctgcaggaaggcatggaagttaaaggtatcgttaagaacctcactgactacggtgcattcgttgatctgggcggcgttgacggcctgctgcacatcactgacatggcctggaaacgcgttaagcatccgagcgaaatcgtcaacgtgggcgacgaaatcactgttaaagtgctgaagttcgaccgcgaacgtacccgtgtatccctgggcctgaaacagctgggcgaagatccgctggtgccgcgcggcagcctcgag

SEQ ID NO: 24 - DNA, 293 bp, Artificial Sequence
  S1 D4 [NcoI, XhoI]
ccatggcctgggtagctatcgctaaacgttatccggaaggtaccaaactgactggtcgcgtgaccaacctgaccgactacggctgcttcgttgaaatcgaagaaggcgttgaaggcctggtacacgtttccgaaatggactggaccaacaaaaacatccacccgtccaaagttgttaacgttggcgatgtagtggaagttatggttctggatatcgacgaagaacgtcgtcgtatctccctgggtctgaaacagtgcaaagctaacccgctggtgccgcgcggcagcctcgag

SEQ ID NO: 25 - DNA, 293 bp, Artificial Sequence
  S1 D5 [NcoI, XhoI]
ccatggcctggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgctggtgccgcgcggcagcctcgag

SEQ ID NO: 26 - DNA, 296 bp, Artificial Sequence
  S1 D6 [NcoI, XhoI]
ccatggccttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaactggtgccgcgcggcagcctcgag

SEQ ID NO: 27 - DNA, 281 bp, Artificial Sequence
  S1 domain of PNPase [NcoI, XhoI]
ccatggcagaaatcgaagtgggccgcgtctacactggtaaagtgacccgtatcgttgactttggcgcatttgttgccatcggcggcggtaaagaaggtctggtccacatctctcaaatcgctgacaaacgcgttgagaaagtgaccgattacctgcagatgggtcaggaagtaccggtgaaagttctggaagttgatcgccagggccgtatccgtctgagcattaaagaagcgactgagcagtctcaacctgctgcactggtgccgcgcggcagcctcgag

SEQ ID NO: 28 - DNA, 1097 bp, Artificial Sequence
  pET28 ARH1 [NcoI, XhoI]
ccatggaaaaatacgtcgccgcgatggttttgtcagctgctggcgatgctttgggatattataatggaaagtgggaatttcttcaggacggggagaaaattcatcgtcaactggctcaattaggggggctggatgctctggacgttggccgttggcgtgtgtctgatgatactgtcatgcacttggcaacagccgaggctttggtcgaggccggaaaggctccaaaactgactcagctttattatttgttagccaagcactatcaggattgcatggaagatatggacggtcgcgcacccgggggtgcgtctgtacacaacgcgatgcagcttaaacctgggaaaccgaatggctggcgtatcccatttaactcgcatgaaggagggtgtggcgcggcgatgcgcgcgatgtgtatcggtttgcgttttccgcatcactctcaattagacacactgatccaagtatcgatcgagtcaggacgtatgacccatcatcacccgacagggtaccttggcgcacttgcgtccgccttattcacggcctatgcggtaaatagccgccctccattgcagtggggtaagggacttatggagcttttgccagaggctaaaaaatacattgtccaatccgggtactttgtggaagaaaatttacagcattggtcttattttcaaacgaagtgggaaaactatcttaaactgcgtggaatcttggacggcgagagtgctccaacattccctgaatcttttggcgttaaagagcgcgaccagttctacacttcgttgtcatatagtggctggggcggttcatctgggcatgatgcccccatgatcgcgtatgacgcggtgctggcggcgggagactcctggaaagagcttgcgcaccgcgccttctttcacggaggtgactcggattcgaccgcagccattgctggatgttggtggggcgtcatgtacggatttaagggcgtcagccccagcaactacgaaaaattagagtatcgcaatcgccttgaggaaacagctcgcgcactttactcgctgggtagtaaagaagacactgttatctcgctgctggtgccgcgcggcagcctcgag

SEQ ID NO: 29 - DNA, 1140 bp, Artificial Sequence
  pTAC ARH1 [XhoI, SphI]
atgaagcttcctcgagaaaaatacgtcgccgcgatggttttgtcagctgctggcgatgctttgggatattataatggaaagtgggaatttcttcaggacggggagaaaattcatcgtcaactggctcaattaggggggctggatgctctggacgttggccgttggcgtgtgtctgatgatactgtcatgcacttggcaacagccgaggctttggtcgaggccggaaaggctccaaaactgactcagctttattatttgttagccaagcactatcaggattgcatggaagatatggacggtcgcgcacccgggggtgcgtctgtacacaacgcgatgcagcttaaacctgggaaaccgaatggctggcgtatcccatttaactcgcatgaaggagggtgtggcgcggcgatgcgcgcgatgtgtatcggtttgcgttttccgcatcactctcaattagacacactgatccaagtatcgatcgagtcaggacgtatgacccatcatcacccgacagggtaccttggcgcacttgcgtccgccttattcacggcctatgcggtaaatagccgccctccattgcagtggggtaagggacttatggagcttttgccagaggctaaaaaatacattgtccaatccgggtactttgtggaagaaaatttacagcattggtcttattttcaaacgaagtgggaaaactatcttaaactgcgtggaatcttggacggcgagagtgctccaacattccctgaatcttttggcgttaaagagcgcgaccagttctacacttcgttgtcatatagtggctggggcggttcatctgggcatgatgcccccatgatcgcgtatgacgcggtgctggcggcgggagactcctggaaagagcttgcgcaccgcgccttctttcacggaggtgactcggattcgaccgcagccattgctggatgttggtggggcgtcatgtacggatttaagggcgtcagccccagcaactacgaaaaattagagtatcgcaatcgccttgaggaaacagctcgcgcactttactcgctgggtagtaaagaagacactgttatctcgctgctggtagtaaagaagacactgttatctcgctgctggtgccgcgcggcagctgcatgc

SEQ ID NO: 30 - DNA, 1097 bp, Artificial Sequence
  pET ARH1 D55,56A [NcoI, XhoI]
ccatggaaaaatacgtcgccgcgatggttttgtcagctgctggcgatgctttgggatattataatggaaagtgggaatttcttcaggacggggagaaaattcatcgtcaactggctcaattaggggggctggatgctctggacgttggccgttggcgtgtgtctgcggcgactgtcatgcacttggcaacagccgaggctttggtcgaggccggaaaggctccaaaactgactcagctttattatttgttagccaagcactatcaggattgcatggaagatatggacggtcgcgcacccgggggtgcgtctgtacacaacgcgatgcagcttaaacctgggaaaccgaatggctggcgtatcccatttaactcgcatgaaggagggtgtggcgcggcgatgcgcgcgatgtgtatcggtttgcgttttccgcatcactctcaattagacacactgatccaagtatcgatcgagtcaggacgtatgacccatcatcacccgacagggtaccttggcgcacttgcgtccgccttattcacggcctatgcggtaaatagccgccctccattgcagtggggtaagggacttatggagcttttgccagaggctaaaaaatacattgtccaatccgggtactttgtggaagaaaatttacagcattggtcttattttcaaacgaagtgggaaaactatcttaaactgcgtggaatcttggacggcgagagtgctccaacattccctgaatcttttggcgttaaagagcgcgaccagttctacacttcgttgtcatatagtggctggggcggttcatctgggcatgatgcccccatgatcgcgtatgacgcggtgctggcggcgggagactcctggaaagagcttgcgcaccgcgccttctttcacggaggtgactcggattcgaccgcagccattgctggatgttggtggggcgtcatgtacggatttaagggcgtcagccccagcaactacgaaaaattagagtatcgcaatcgccttgaggaaacagctcgcgcactttactcgctgggtagtaaagaagacactgttatctcgctgctggtgccgcgcggcagcctcgag

SEQ ID NO: 31 - DNA, 1108 bp, Artificial Sequence
  pTAC ARH1 D55,56A [XhoI, SphI]
atgaagcttcctcgagaaaaatacgtcgccgcgatggttttgtcagctgctggcgatgctttgggatattataatggaaagtgggaatttcttcaggacggggagaaaattcatcgtcaactggctcaattaggggggctggatgctctggacgttggccgttggcgtgtgtctgcggcgactgtcatgcacttggcaacagccgaggctttggtcgaggccggaaaggctccaaaactgactcagctttattatttgttagccaagcactatcaggattgcatggaagatatggacggtcgcgcacccgggggtgcgtctgtacacaacgcgatgcagcttaaacctgggaaaccgaatggctggcgtatcccatttaactcgcatgaaggagggtgtggcgcggcgatgcgcgcgatgtgtatcggtttgcgttttccgcatcactctcaattagacacactgatccaagtatcgatcgagtcaggacgtatgacccatcatcacccgacagggtaccttggcgcacttgcgtccgccttattcacggcctatgcggtaaatagccgccctccattgcagtggggtaagggacttatggagcttttgccagaggctaaaaaatacattgtccaatccgggtactttgtggaagaaaatttacagcattggtcttattttcaaacgaagtgggaaaactatcttaaactgcgtggaatcttggacggcgagagtgctccaacattccctgaatcttttggcgttaaagagcgcgaccagttctacacttcgttgtcatatagtggctggggcggttcatctgggcatgatgcccccatgatcgcgtatgacgcggtgctggcggcgggagactcctggaaagagcttgcgcaccgcgccttctttcacggaggtgactcggattcgaccgcagccattgctggatgttggtggggcgtcatgtacggatttaagggcgtcagccccagcaactacgaaaaattagagtatcgcaatcgccttgaggaaacagctcgcgcactttactcgctgggtagtaaagaagacactgttatctcgctgctggtgccgcgcggcagctgcatgc

SEQ ID NO: 32 - DNA, 39 bp, Artificial Sequence
  Fwd Alt NcoI
atcgacccatgggagaacttattacagaattatttgacg

SEQ ID NO: 33 - DNA, 55 bp, Artificial Sequence
  Rev Alt XhoI
attcgactcgaggctgccgcgcggcaccagtccttgaacgaacttgtaaggcatg

SEQ ID NO: 34 - DNA, 38 bp, Artificial Sequence
  Fwd ModA NcoI
atcgaccatgggaaaatactcagtaatgcaactaaaag

SEQ ID NO: 35 - DNA, 54 bp, Artificial Sequence
  Rev ModA XhoI
atcgtactcgaggctgccgcgcggcaccagtagattaaatccttcaaaatcaag

SEQ ID NO: 36 - DNA, 36 bp, Artificial Sequence
  Fwd ModB NcoI
atcgacccatgggaattattaatcttgcagatgttg

SEQ ID NO: 37 - DNA, 54 bp, Artificial Sequence
  Rev ModB XhoI
acttagctcgaggctgccgcgcggcaccagttcaactacctcaatatcataaac

SEQ ID NO: 38 - DNA, 47 bp, Artificial Sequence
  Fwd rS1 NcoI
atcgacccatgggaactgaatcttttgctcaactctttgaagagtcc

SEQ ID NO: 39 - DNA, 49 bp, Artificial Sequence
  Rev rS1 XhoI
attcgactcgaggctgccgcgcggcaccagctcgcctttagctgctttg

SEQ ID NO: 40 - DNA, 51 bp, Artificial Sequence
  Fwd rS1-pTAC XhoI
atgaagcttcctcgagagactgaatcttttgctcaactctttgaagagtcc

SEQ ID NO: 41 - DNA, 46 bp, Artificial Sequence
  Rev rS1-pTAC SphI
ctctacgtgcatgcactcgcctttagctgctttgaaagcttcagcc

SEQ ID NO: 42 - DNA, 43 bp, Artificial Sequence
  Fwd NcoI rS1 D1
atcgacccatggagtccttaaaagaaatcgaaacccgcccggg

SEQ ID NO: 43 - DNA, 58 bp, Artificial Sequence
  Rev XhoI rS1 D1
tggtgctcgaggctgccgcgcggcaccagggcttcgtgacgtttagctttctcacggg

SEQ ID NO: 44 - DNA, 52 bp, Artificial Sequence
  Fwd NcoI rS1 D2
atcgacccatggcctggatcacgctggaaaaagcttacgaagatgctgaaac

SEQ ID NO: 45 - DNA, 60 bp, Artificial Sequence
  Rev XhoI rS1 D2
ggtgctcgaggctgccgcgcggcaccagctctgcgctgttttcggattcgataacggcac

SEQ ID NO: 46 - DNA, 46 bp, Artificial Sequence
  Fwd NcoI rS1 D3
atcgacccatggcccgcgatcagctgctggaaaacctgcaggaagg

SEQ ID NO: 47 - DNA, 60 bp, Artificial Sequence
  Rev XhoI rS1 D3
tggtgctcgaggctgccgcgcggcaccagcggatcttcgcccagctgtttcaggcccagg

SEQ ID NO: 48 - DNA, 46 bp, Artificial Sequence
  Fwd NcoI rS1 D4
atcgacccatggcctgggtagctatcgctaaacgttatccggaagg

SEQ ID NO: 49 - DNA, 63 bp, Artificial Sequence
  Rev XhoI rS1 D4
tggtgctcgaggctgccgcgcggcaccagcgggttagctttgcactgtttcagacccagggag

SEQ ID NO: 50 - DNA, 57 bp, Artificial Sequence
  Fwd NcoI rS1 D5
atcgacccatggcctggcagcagttcgcggaaacccacaacaagggcgaccgtgttg

SEQ ID NO: 51 - DNA, 68 bp, Artificial Sequence
  Rev XhoI S1 D5
tggtgctcgaggctgccgcgcggcaccagcggatcttctgcgagctgtttaacgcccagggagatacg

SEQ ID NO: 52 - DNA, 54 bp, Artificial Sequence
  Fwd NcoI rS1 D6
atcgacccatggccttcaacaactgggttgctctgaacaagaaaggcgctatcg

SEQ ID NO: 53 - DNA, 66 bp, Artificial Sequence
  Rev XhoI rS1 D6
tggtgctcgaggctgccgcgcggcaccagtttctcgtcagcttcgtctttcgcacgaacagacagg

SEQ ID NO: 54 - DNA, 55 bp, Artificial Sequence
  Fwd NcoI PNPase rS1 binding
atcgacccatggcagaaatcgaagtgggccgcgtctacactggtaaagtgacccg

SEQ ID NO: 55 - DNA, 59 bp, Artificial Sequence
  Rev XhoI PNPase rS1 binding
tggtgctcgaggctgccgcgcggcaccagtgcagcaggttgagactgctcagtcgcttc

SEQ ID NO: 56 - DNA, 31 bp, Artificial Sequence
  Fwd ARH1 NcoI
tgcagccatggaaaaatacgtcgccgcgatg

SEQ ID NO: 57 - DNA, 30 bp, Artificial Sequence
  Rev ARH1 XhoI
gtggtgctcgaggctgccgcgcggcaccag

SEQ ID NO: 58 - DNA, 54 bp, Artificial Sequence
  Fwd XhoI pTAC ARH1
atgaagcttcctcgagaaaaatacgtcgccgcgatggttttgtcagctgctggc

SEQ ID NO: 59 - DNA, 61 bp, Artificial Sequence
  Rev SphI pTAC ARH1
ctacgtgcatgcagctgccgcgcggcaccagcagcgagataacagtgtcttctttactacc

SEQ ID NO: 60 - DNA, 31 bp, Artificial Sequence
  Fwd rS1 R139A
ctggtagacgttgccccggtgcgtgacactc

SEQ ID NO: 61 - DNA, 31 bp, Artificial Sequence
  Fwd rS1 R139K
ctggtagacgttaaaccggtgcgtgacactc

SEQ ID NO: 62 - DNA, 28 bp, Artificial Sequence
  Rev rS1 R139
agaacctggcaggaacgcacgaataccg

SEQ ID NO: 63 - DNA, 41 bp, Artificial Sequence
  Fwd ARH1 D55,56A
ggccgttggcgtgtgtctgcggcgactgtcatgcacttggc

SEQ ID NO: 64 - DNA, 27 bp, Artificial Sequence
  Rev ARH1 D55,56A
aacgtccagagcatccagcccccctaa

SEQ ID NO: 65 - protein, 12 aa, Artificial Sequence
  Fragment
FLPGSLVDVRPV

SEQ ID NO: 66 - protein, 18 aa, Artificial Sequence
  rS1 peptide
KGDEIAAVVLQVDAERER

SEQ ID NO: 67 - protein, 21 aa, Artificial Sequence
  rS1 peptide
AFLPGSLVDVRPVRDTHLEGK

SEQ ID NO: 68 - protein, 14 aa, Artificial Sequence
  rS1 peptide
AFLPGSLVDVAPVR

SEQ ID NO: 69 - protein, 14 aa, Artificial Sequence
  rS1 peptide
AFLPGSLVDVKPVR

SEQ ID NO: 70 - protein, 87 aa, Artificial Sequence
  s1d2
WITLEKAYEDAETVTGVINGKVKGGFTVELNGIRAFLPGSLVDVRPVRDTLHLEGKELEFKVIKLDQKRNNVVVSRRAVIESENSAE

SEQ ID NO: 71 - protein, 88 aa, Artificial Sequence
  s1d6
FNNWVALNKKGAIVTGKVTAVDAKGATVELADGVEGYLRASEASRDRVEDATLVLSVGDEVEAKFTGVDRKNRAISLSVRAKDEADEK

SEQ ID NO: 72 - protein, 84 aa, Artificial Sequence
  polynucleotide
AEIEVGRVYTGKVTRIVDFGAFVAIGGGKEGLVHISQIADKRVEKVTDYLQMGQEVPVKVLEVDRQGRIRLSIKEATEQSQPAA

SEQ ID NO: 73 - RNA, 10 nt, Artificial Sequence
  5′P-A-10mer-Cy5
agacuucgac

SEQ ID NO: 74 - RNA, 10 nt, Artificial Sequence
  5′P-G-10mer-Cy5
ggcauucgac

SEQ ID NO: 75 - RNA, 10 nt, Artificial Sequence
  5′P-C-10mer-Cy5
cgcauucgac

SEQ ID NO: 76 - RNA, 10 nt, Artificial Sequence
  5′P-U-10mer-Cy5
ugcauucgac